(19) United States
(12) Patent Application Publication
(10) Pub. No.: US 2025/0376510 A1
(43) Pub. Date: Dec. 11, 2025

(54) ANTI-SURVIVIN ANTIBODIES AND USES THEREOF

(71) Applicant: MIMIVAX, INC., Buffalo, NY (US)

(72) Inventors: Michael J. CIESIELSKI, Orchard Park, NY (US); Robert A. FENSTERMAKER, Clarence, NY (US)

(21) Appl. No.: 19/220,489

(22) Filed: May 28, 2025

Related U.S. Application Data
(60) Provisional application No. 63/652,523, filed on May 28, 2024.

(51) Int. Cl. C07K 16/18; A61K 39/00; A61P 21/04; A61P 35/00

(52) U.S. Cl. CPC C07K 16/18; A61P 21/04; A61P 35/00; A61K 2039/505; A61K 2039/54; C07K 2317/569; C07K 2317/622; C07K 2317/732; C07K 2317/734; C07K 2317/92

(57) ABSTRACT

Provided herein are antibodies, and antibody fragments thereof, that bind human survivin. Also provided herein are isolated nucleic acids, expression vectors, host cells, pharmaceutical compositions, and kits that relate to the antibodies and antibody fragments. Finally, methods of treating survivin-mediated disorders are also provided, such as various autoimmune disorders and cancer.

### CROSS-REFERENCE TO RELATED APPLICATIONS

This application claims priority to U.S. Provisional Application No. 63/652,523, filed on May 28, 2024, which is hereby incorporated by reference in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “260600000101 Sequence Listing.xml”, was created on May 28, 2025, and is 122,880 bytes in size.

### BACKGROUND

Myasthenia gravis (MG) is an autoimmune disorder characterized by muscle weakness, primarily caused by autoantibodies targeting the muscle acetylcholine receptor and the muscle-specific kinase at the neuromuscular junction. Although the pathophysiology of the disease is understood, the B-cell subset responsible for autoantibody production has not been fully identified, and no curative treatments for MG are available. Recent studies have suggested a potential role for survivin, a protein implicated in cell survival and proliferation, in the pathogenesis of MG.

Despite the potential role of survivin, no current MG treatments can specifically target the cells that produce these specific autoantibodies, and current treatments generally exhibit limited efficacy and/or present adverse effect that negatively impact quality of life. Existing therapies, such as corticosteroids and immunomodulators, act systemically and are symptomatic, with severe side effects, including an increased risk of infection, bone thinning, and gastrointestinal disturbances. New therapies, including antibodies against the complement and neonatal Fc receptor, as well as B-cell-targeted immunotherapy, aim to reduce autoantibodies and hold promise as valuable tools in treating MG. However, these therapies do not specifically target the pathogenic cells or the antigen-specific B-cell; related clinical trials have shown that some subjects respond poorly or not at all. The potential involvement of survivin in MG pathogenesis suggests a novel therapeutic approach that could specifically target the pathogenic cells.

Beyond autoimmune disorders, survivin is implicated in the survival and proliferation of cancer cells and has emerged as a target in oncology. Survivin's role in promoting cell survival by inhibiting apoptosis makes it a potent target for cancer therapeutics, as its expression is typically associated with aggressive and treatment-resistant tumors. The involvement of survivin in both cancer and autoimmune diseases provides a unique therapeutic opportunity.

### SUMMARY OF THE DISCLOSURE

Provided herein are antibodies, or antigen-binding fragments thereof, comprising a heavy chain variable domain (VHH) comprising a complementarity-determining region (CDR) 1, a CDR2, and a CDR3, wherein:
i. CDR1 comprises the amino acid sequence of SEQ ID NO: 1, CDR2 comprises the amino acid sequence of SEQ ID NO: 2, and CDR3 comprises the amino acid sequence of SEQ ID NO: 3;
ii. CDR1 comprises the amino acid sequence of SEQ ID NO: 5, CDR2 comprises the amino acid sequence of SEQ ID NO: 6, and CDR3 comprises the amino acid sequence of SEQ ID NO: 7;
iii. CDR1 comprises the amino acid sequence of SEQ ID NO: 9, CDR2 comprises the amino acid sequence of SEQ ID NO: 2, and CDR3 comprises the amino acid sequence of SEQ ID NO: 10;
iv. CDR1 comprises the amino acid sequence of SEQ ID NO: 9, CDR2 comprises the amino acid sequence of SEQ ID NO: 12, and CDR3 comprises the amino acid sequence of SEQ ID NO: 13;
v. CDR1 comprises the amino acid sequence of SEQ ID NO: 15, CDR2 comprises the amino acid sequence of SEQ ID NO: 12, and CDR3 comprises the amino acid sequence of SEQ ID NO: 13;
vi. CDR1 comprises the amino acid sequence of SEQ ID NO: 1, CDR2 comprises the amino acid sequence of SEQ ID NO: 17, and CDR3 comprises the amino acid sequence of SEQ ID NO: 7;
vii. CDR1 comprises the amino acid sequence of SEQ ID NO: 1, CDR2 comprises the amino acid sequence of SEQ ID NO: 6, and CDR3 comprises the amino acid sequence of SEQ ID NO: 7;
viii. CDR1 comprises the amino acid sequence of SEQ ID NO: 1, CDR2 comprises the amino acid sequence of SEQ ID NO: 20, and CDR3 comprises the amino acid sequence of SEQ ID NO: 21;
ix. CDR1 comprises the amino acid sequence of SEQ ID NO: 23, CDR2 comprises the amino acid sequence of SEQ ID NO: 6, and CDR3 comprises the amino acid sequence of SEQ ID NO: 24;
x. CDR1 comprises the amino acid sequence of SEQ ID NO: 9, CDR2 comprises the amino acid sequence of SEQ ID NO: 26, and CDR3 comprises the amino acid sequence of SEQ ID NO: 27;
xi. CDR1 comprises the amino acid sequence of SEQ ID NO: 9, CDR2 comprises the amino acid sequence of SEQ ID NO: 6, and CDR3 comprises the amino acid sequence of SEQ ID NO: 29;
xii. CDR1 comprises the amino acid sequence of SEQ ID NO: 31, CDR2 comprises the amino acid sequence of SEQ ID NO: 32, and CDR3 comprises the amino acid sequence of SEQ ID NO: 33;
xiii. CDR1 comprises the amino acid sequence of SEQ ID NO: 35, CDR2 comprises the amino acid sequence of SEQ ID NO: 36, and CDR3 comprises the amino acid sequence of SEQ ID NO: 37;
xiv. CDR1 comprises the amino acid sequence of SEQ ID NO: 39, CDR2 comprises the amino acid sequence of SEQ ID NO: 40, and CDR3 comprises the amino acid sequence of SEQ ID NO: 37;
xv. CDR1 comprises the amino acid sequence of SEQ ID NO: 35, CDR2 comprises the amino acid sequence of SEQ ID NO: 42, and CDR3 comprises the amino acid sequence of SEQ ID NO: 37;
xvi. CDR1 comprises the amino acid sequence of SEQ ID NO: 39, CDR2 comprises the amino acid sequence of SEQ ID NO: 42, and CDR3 comprises the amino acid sequence of SEQ ID NO: 37;
xvii. CDR1 comprises the amino acid sequence of SEQ ID NO: 39, CDR2 comprises the amino acid sequence of SEQ ID NO: 40, and CDR3 comprises the amino acid sequence of SEQ ID NO: 45;
xviii. CDR1 comprises the amino acid sequence of SEQ ID NO: 47, CDR2 comprises the amino acid sequence of SEQ ID NO: 42, and CDR3 comprises the amino acid sequence of SEQ ID NO: 45;
xix. CDR1 comprises the amino acid sequence of SEQ ID NO: 49, CDR2 comprises the amino acid sequence of SEQ ID NO: 42, and CDR3 comprises the amino acid sequence of SEQ ID NO: 45;
xx. CDR1 comprises the amino acid sequence of SEQ ID NO: 51, CDR2 comprises the amino acid sequence of SEQ ID NO: 42, and CDR3 comprises the amino acid sequence of SEQ ID NO: 45;
xxi. CDR1 comprises the amino acid sequence of SEQ ID NO: 53, CDR2 comprises the amino acid sequence of SEQ ID NO: 54, and CDR3 comprises the amino acid sequence of SEQ ID NO: 55;
xxii. CDR1 comprises the amino acid sequence of SEQ ID NO: 57, CDR2 comprises the amino acid sequence of SEQ ID NO: 54, and CDR3 comprises the amino acid sequence of SEQ ID NO: 55;
xxiii. CDR1 comprises the amino acid sequence of SEQ ID NO: 9, CDR2 comprises the amino acid sequence of SEQ ID NO: 54, and CDR3 comprises the amino acid sequence of SEQ ID NO: 55;
xxiv. CDR1 comprises the amino acid sequence of SEQ ID NO: 60, CDR2 comprises the amino acid sequence of SEQ ID NO: 42, and CDR3 comprises the amino acid sequence of SEQ ID NO: 55;
xxv. CDR1 comprises the amino acid sequence of SEQ ID NO: 62, CDR2 comprises the amino acid sequence of SEQ ID NO: 42, and CDR3 comprises the amino acid sequence of SEQ ID NO: 55;
xxvi. CDR1 comprises the amino acid sequence of SEQ ID NO: 64, CDR2 comprises the amino acid sequence of SEQ ID NO: 42, and CDR3 comprises the amino acid sequence of SEQ ID NO: 55;
xxvii. CDR1 comprises the amino acid sequence of SEQ ID NO: 47, CDR2 comprises the amino acid sequence of SEQ ID NO: 42, and CDR3 comprises the amino acid sequence of SEQ ID NO: 55;
xxviii. CDR1 comprises the amino acid sequence of SEQ ID NO: 35, CDR2 comprises the amino acid sequence of SEQ ID NO: 42, and CDR3 comprises the amino acid sequence of SEQ ID NO: 55;
xxix. CDR1 comprises the amino acid sequence of SEQ ID NO: 68, CDR2 comprises the amino acid sequence of SEQ ID NO: 42, and CDR3 comprises the amino acid sequence of SEQ ID NO: 55;
xxx. CDR1 comprises the amino acid sequence of SEQ ID NO: 49, CDR2 comprises the amino acid sequence of SEQ ID NO: 42, and CDR3 comprises the amino acid sequence of SEQ ID NO: 55;
xxxi. CDR1 comprises the amino acid sequence of SEQ ID NO: 1, CDR2 comprises the amino acid sequence of SEQ ID NO: 20, and CDR3 comprises the amino acid sequence of SEQ ID NO: 71;
xxxii. CDR1 comprises the amino acid sequence of SEQ ID NO: 73, CDR2 comprises the amino acid sequence of SEQ ID NO: 74, and CDR3 comprises the amino acid sequence of SEQ ID NO: 75;
xxxiii. CDR1 comprises the amino acid sequence of SEQ ID NO: 77, CDR2 comprises the amino acid sequence of SEQ ID NO: 78, and CDR3 comprises the amino acid sequence of SEQ ID NO: 79;
xxxiv. CDR1 comprises the amino acid sequence of SEQ ID NO: 81, CDR2 comprises the amino acid sequence of SEQ ID NO: 82, and CDR3 comprises the amino acid sequence of SEQ ID NO: 83;
xxxv. CDR1 comprises the amino acid sequence of SEQ ID NO: 31, CDR2 comprises the amino acid sequence of SEQ ID NO: 85, and CDR3 comprises the amino acid sequence of SEQ ID NO: 86;
xxxvi. CDR1 comprises the amino acid sequence of SEQ ID NO: 88, CDR2 comprises the amino acid sequence of SEQ ID NO: 78, and CDR3 comprises the amino acid sequence of SEQ ID NO: 89;
xxxvii. CDR1 comprises the amino acid sequence of SEQ ID NO: 91, CDR2 comprises the amino acid sequence of SEQ ID NO: 92, and CDR3 comprises the amino acid sequence of SEQ ID NO: 93;
xxxviii. CDR1 comprises the amino acid sequence of SEQ ID NO: 95, CDR2 comprises the amino acid sequence of SEQ ID NO: 96, and CDR3 comprises the amino acid sequence of SEQ ID NO: 97;
xxxix. CDR1 comprises the amino acid sequence of SEQ ID NO: 99, CDR2 comprises the amino acid sequence of SEQ ID NO: 100, and CDR3 comprises the amino acid sequence of SEQ ID NO: 101; or
xl. CDR1 comprises the amino acid sequence of SEQ ID NO: 103, CDR2 comprises the amino acid sequence of SEQ ID NO: 104, and CDR3 comprises the amino acid sequence of SEQ ID NO: 105.

In some embodiments, the VHH comprises an amino acid sequence having at least 90%, 91%, 92%, 93%, 94%, 95%, 96%, 97%, 98%, or 99% sequence identity to SEQ ID NO: 4, SEQ ID NO: 8, SEQ ID NO: 11, SEQ ID NO: 14, SEQ ID NO: 16, SEQ ID NO: 18, SEQ ID NO: 19, SEQ ID NO: 22, SEQ ID NO: 25, SEQ ID NO: 28, SEQ ID NO: 30, SEQ ID NO: 34, SEQ ID NO: 38, SEQ ID NO: 41, SEQ ID NO: 43, SEQ ID NO: 44, SEQ ID NO: 46, SEQ ID NO: 48, SEQ ID NO: 50, SEQ ID NO: 52, SEQ ID NO: 56, SEQ ID NO: 58, SEQ ID NO: 59, SEQ ID NO: 61, SEQ ID NO: 63, SEQ ID NO: 65, SEQ ID NO: 66, SEQ ID NO: 67, SEQ ID NO: 69, SEQ ID NO: 70, SEQ ID NO: 72, SEQ ID NO: 76, SEQ ID NO: 80, SEQ ID NO: 84, SEQ ID NO: 87, SEQ ID NO: 90, SEQ ID NO: 94, SEQ ID NO: 98, SEQ ID NO: 102, or SEQ ID NO: 106, provided that the VHH comprises the sequences of at least one set of CDR1, CDR2, and CDR3 sequences of claim 1.

In some embodiments, the VHH comprises an amino acid sequence identical to SEQ ID NO: 4, SEQ ID NO: 8, SEQ ID NO: 11, SEQ ID NO: 14, SEQ ID NO: 16, SEQ ID NO: 18, SEQ ID NO: 19, SEQ ID NO: 22, SEQ ID NO: 25, SEQ ID NO: 28, SEQ ID NO: 30, SEQ ID NO: 34, SEQ ID NO: 38, SEQ ID NO: 41, SEQ ID NO: 43, SEQ ID NO: 44, SEQ ID NO: 46, SEQ ID NO: 48, SEQ ID NO: 50, SEQ ID NO: 52, SEQ ID NO: 56, SEQ ID NO: 58, SEQ ID NO: 59, SEQ ID NO: 61, SEQ ID NO: 63, SEQ ID NO: 65, SEQ ID NO: 66, SEQ ID NO: 67, SEQ ID NO: 69, SEQ ID NO: 70, SEQ ID NO: 72, SEQ ID NO: 76, SEQ ID NO: 80, SEQ ID NO: 84, SEQ ID NO: 87, SEQ ID NO: 90, SEQ ID NO: 94, SEQ ID NO: 98, SEQ ID NO: 102, or SEQ ID NO: 106.

In some embodiments, the VHH comprises an amino acid sequence having at least 90%, 91%, 92%, 93%, 94%, 95%, 96%, 97%, 98%, or 99% sequence identity, or is identical to, SEQ ID NO: 22, provided that CDR1 comprises the amino acid sequence of SEQ ID NO: 1, CDR2 comprises the amino acid sequence of SEQ ID NO: 20, and CDR3 comprises the amino acid sequence of SEQ ID NO: 21.

In some embodiments, the VHH comprises an amino acid sequence having at least 90%, 91%, 92%, 93%, 94%, 95%, 96%, 97%, 98%, or 99% sequence identity, or is identical to, SEQ ID NO: 43, provided that CDR1 comprises the amino acid sequence of SEQ ID NO: 35, CDR2 comprises the amino acid sequence of SEQ ID NO: 42, and CDR3 comprises the amino acid sequence of SEQ ID NO: 37.

In some embodiments, the VHH comprises an amino acid sequence having at least 90%, 91%, 92%, 93%, 94%, 95%, 96%, 97%, 98%, or 99% sequence identity, or is identical to, SEQ ID NO: 72, provided that CDR1 comprises the amino acid sequence of SEQ ID NO: 1, CDR2 comprises the amino acid sequence of SEQ ID NO: 20, and CDR3 comprises the amino acid sequence of SEQ ID NO: 71.

In some embodiments, the antibody, or antigen-binding fragment thereof is a single domain antibody (sdAb), a nanobody, or an isolated VHH domain.

In some embodiments, the antibody or antigen-binding fragment thereof binds to human survivin. In some embodiments, the antibody or antigen-binding fragment thereof binds to human survivin on the surface of B-cells.

Also provided is an isolated nucleic acid molecule encoding any antibody, or antigen-binding fragment thereof, disclosed herein.

Also provided is an expression vector comprising any nucleic acid molecule disclosed herein.

Also provided is a host cell comprising any nucleic acid molecule disclosed herein or any expression vector disclosed herein.

Also provided is a pharmaceutical composition comprising any antibody, or antigen-binding fragment thereof, disclosed herein. In some embodiments, the pharmaceutical composition is formulated for intravenous or subcutaneous injection. In some embodiments, the pharmaceutical composition is an injectable pharmaceutical composition.

Also provided is a kit comprising: a) any antibody, or antibody binding fragment disclosed herein, b) any isolated nucleic acid molecule disclosed herein, c) any expression vector disclosed herein, and/or d) any pharmaceutical composition disclosed herein, and packaging for the same.

Also provided is a method of producing an antibody, or antigen-binding fragment thereof, that binds human survivin, the method comprising: (a) growing or culturing the host cell of claim 12 under conditions so that the host cell expresses a polypeptide or polypeptides comprising a heavy chain variable domain (VHH), thereby producing the antibody or the antigen-binding fragment of the antibody; and (b) purifying the antibody, or the antigen-binding fragment thereof.

Also provided is a method of treating a survivin-mediated disorder in a subject in need thereof, the method comprising administering to the subject any antibody, or antigen-binding fragment thereof, disclosed herein or any pharmaceutical composition disclosed herein.

In some embodiments, the survivin-mediated disorder is an autoimmune disorder. In some embodiments, the autoimmune disorder is or comprises antineutrophil cytoplasmic antibody (ANCA)-associated vasculitis, alopecia areata, ankylosing spondylitis, antiphospholipid syndrome, Addison's disease, hemolytic anemia, autoimmune hepatitis, hepatitis, Behcets disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, systemic lupus erythematosus, Meniere's Disease, mixed connective tissue disease, multiple sclerosis, myasthenia gravis, neuromyelitis optica, pemphigus vulgaris, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjogren's syndrome, stiff-man syndrome, systemic sclerosis, Takayasu arteritis, temporal arteritis, ulcerative colitis, uveitis, vitiligo, and Wegener's granulomatosis. In some embodiments, the autoimmune disorder is myasthenia gravis.

In some embodiments, the survivin-mediated disorder is a cancer. In some embodiments, the cancer is or comprises breast cancer, kidney cancer, liver cancer, lung cancer, malignant glioma, melanoma, multiple myeloma, neuroendocrine tumors (NETs), pediatric brain tumors (gliomas), and prostate cancer.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1A depicts fluorescence-activated cell sorting (FACS) analysis showing total survivin expression in peripheral blood mononuclear cells (PBMCs) from a myasthenic patient.

FIG. 1B depicts FACS analysis of total survivin expression in PBMCs from a healthy control.

FIG. 1C depicts a representative image of hematoxylin and eosin (H&E) staining in the thymus of a 24-year-old female patient with early-onset myasthenia gravis (MG).

FIG. 1D depicts survivin-positive staining in the thymus of a 24-year-old female patient with early-onset MG.

FIG. 1E depicts the binding of the a subunit of acetylcholine receptor (AChR) peptide to PBMCs from a healthy control, determined by five-color FACS analysis.

FIG. 1F depicts the binding of the a subunit of AChR peptide to PBMCs from a myasthenic patient, determined by five-color FACS analysis.

FIGS. 2A-2D depict flow cytometry detection of survivin isoforms in HEK293T (FIG. 2A), HeLa (FIG. 2B), U87 (FIG. 2C), and A1207 (FIG. 2D) cells using non-permeabilized conditions, showing expression levels of survivin wild type (svn-wt), dE3X3, and 2B, and each figure includes a corresponding Western blot analysis showing survivin isoform expression levels.

FIG. 3A depicts the titers of anti-survivin specific antibodies (IgG) across different time points throughout the SurVaxM vaccination schedule, showing baseline (pre-immunization), post-fourth vaccination (Post-V4), and subsequent time points V5, V6, and V7.

FIG. 3B depicts the anti-survivin IgG titers in two cohorts categorized by overall survival (OS): one with OS≤14 months and the other with OS>14 months, showing a correlation between higher antibody titers and extended overall survival.

FIG. 4 depicts the mean fluorescence intensity (MFI) values for various anti-SurVaxM monoclonal antibody clones, including MV2C2, across three different volumes (0.5 μl, 3.0 μl, and 5.0 μl), indicating superior binding capability of MV2C2 as compared to control and other clones.

FIG. 5 depicts the binding affinity of the MV2C2 monoclonal antibody to the survivin epitope SVN53-67, with a dissociation constant (KD) of 9.9×10⁻¹⁰ M, demonstrating high affinity suitable for therapeutic use.

FIG. 6 depicts a schematic representation of the mechanisms of action of the MV2C2 antibody. The diagram illustrates the binding of MV2C2 to extracellular survivin on B-cells, resulting in antibody-dependent cellular phagocytosis by macrophages, complement-dependent cytotoxicity involving the C1 complex, and antibody-dependent cellular cytotoxicity mediated by natural killer (NK) cells.

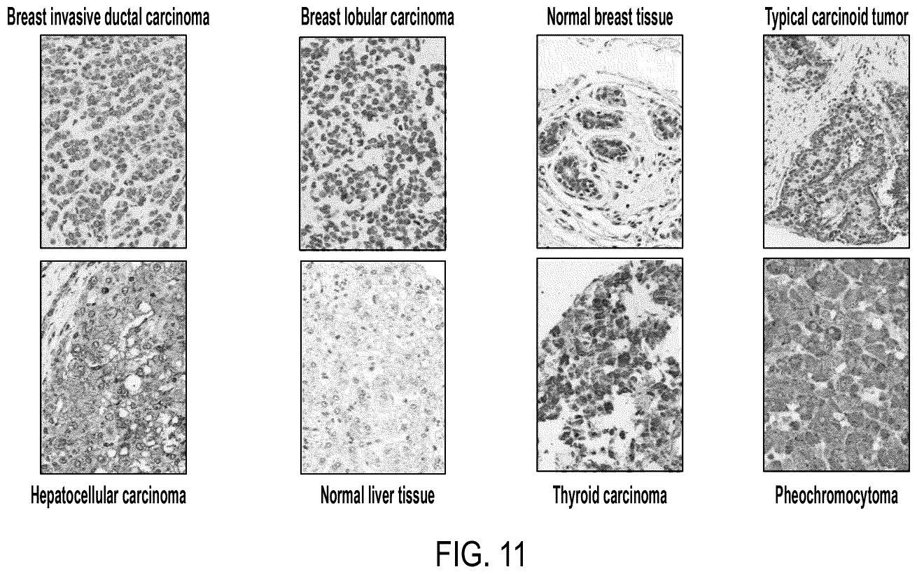

FIG. 7 depicts histological panels showing the reactivity of the MV2C2 antibody across a variety of cancer types with each tissue type stained to show the over-expression of survivin.

FIG. 8 depicts a histological image showing survivin expression in a lower-grade glioma, stained using the MV2C2 antibody.

FIG. 9 depicts survivin expression in multiple myeloma tissues, stained using the MV2C2 antibody.

FIG. 10 depicts a neuroendocrine tumor tissue micro array analysis where survivin expression is identified in a significant fraction of tissues using the MV2C2 antibody.

FIG. 11 depicts survivin expression in various cancer tissues such as breast, liver, and thyroid cancers against their corresponding normal tissues, using MV2C2 staining.

FIG. 12 depicts the effect of different treatments on tumor volume in a GL261 glioma model grown in C57BL/6 mice. The bar graph shows maximum tumor volume through 32 days for control, SurVaxM, PBMCs, antisera, and purified monoclonal antibody (mAb) IgG groups. The purified mAb IgG group exhibits significant tumor growth reduction compared to controls, demonstrating potent anti-tumor activity.

FIG. 13A depicts the tumor growth inhibition effects of mAb 2C2 compared to non-specific IgG across different mouse strains and tumor types, including GL261 glioma and B16F1 melanoma models in C57BL/6 and Nude/Ncr mice. The graphs show reduced tumor volumes with mAb 2C2 treatment over 20 to 30 days.

FIG. 13B depicts the survival curves of C57BL/6 mice inoculated with GL261 cells and treated with non-specific IgG, mAb 2C2, or SurVaxM. The survival analysis shows significantly prolonged survival rates for mice treated with mAb 2C2 compared to non-specific IgG, indicating the therapeutic potential of mAb 2C2.

FIG. 14 depicts flow cytometry analysis evaluating the specificity and efficacy of the MV2C2 monoclonal antibody in binding survivin-expressing cancer cells from various lines.

FIG. 15 depicts the therapeutic efficacy of MV2C2 monoclonal antibody in controlling tumor growth across different cancer models.

FIG. 16 depicts the development and efficacy of CAR-T cell therapies utilizing the MV2C2 monoclonal antibody as a chimeric antigen receptor targeting survivin-expressing glioma cells.

FIG. 17 depicts the overall survival results from a Phase 2a clinical trial of SurVaxM in patients with newly diagnosed glioblastoma.

FIG. 18 depicts a Kaplan-Meier curve from a Phase I trial of SurVaxM in multiple myeloma patients treated with lenalidomide.

FIG. 19 depicts antibody titers measured during the immunization phase of a SurVaxM trial in multiple myeloma patients.

FIG. 20 depicts a Kaplan-Meier curve from a Phase I study of SurVaxM in patients with metastatic neuroendocrine tumors (NETs), displaying overall survival over three years.

FIG. 21 depicts anti-SurVaxM immunogenicity in neuroendocrine tumor patients, represented in two plots comparing baseline and post-treatment IgG titers.

FIG. 22 depicts the process of biopanning rounds for selecting survivin-specific single-domain antibodies (sdAbs) from a naïve phage display library. The diagram outlines three rounds of biopanning using biotinylated peptides: round 1 with wild-type peptide, round 2 with mutated peptide, and round 3 with wild-type peptide.

FIG. 23 depicts the ELISA results for pool S1 following the third round of biopanning (R3) against the biotinylated mutated survivin peptide (1B). The table shows quantitative assessments of binding affinities between selected sdAbs and the survivin-derived peptides, highlighting specific clones with high affinity for the target epitope.

FIG. 24 depicts the ELISA data for pool S1 following the third round of biopanning, using the biotinylated wild-type survivin peptide (1C). The table shows the binding affinities of selected sdAbs to the peptide, highlighting clones with high affinity.

FIG. 25 depicts the control ELISA data for pool S1 using an isotype biotin peptide. This serves as a benchmark to assess the specificity of the antibody-antigen interaction.

FIG. 26 depicts the ELISA data for pool S2 following the third round of biopanning, using the biotinylated mutated survivin peptide (1B). The table displays the binding affinities of selected sdAbs to the peptide, indicating specific clones with high affinity.

FIG. 27 depicts the ELISA data for pool S2 following the third round of biopanning, using the biotinylated wild-type survivin peptide (1C). The table presents the binding affinities of selected sdAbs to the peptide, highlighting clones with significant binding.

FIG. 28 depicts the control ELISA data for pool S2 against the isotype biotin peptide. This control data serves as a benchmark to validate the specificity of the antibody-antigen interaction.

FIG. 29 depicts the ELISA data for pool S3 following the third round of biopanning, using the biotinylated mutated survivin peptide (1B).

FIG. 30 depicts the ELISA data for pool S3 following the third round of biopanning, using the biotinylated wild-type survivin peptide (1C).

FIG. 31 depicts the control ELISA data for pool S3 against the isotype biotin peptide. This control data serves as a benchmark to validate the specificity of the antibody-antigen interaction.

FIG. 32 depicts ELISA absorbance readings at 450 nm for selected sdAb clones from the third round of biopanning. The data delineates the specific interaction levels of the clones with the wild-type and mutant survivin-derived peptides.

FIG. 33 depicts the nucleotide and corresponding amino acid sequences of the complementarity-determining regions (CDRs) of the heavy chain variable domains (VHH) from the selected sdAbs.

FIG. 34 depicts the ELISA data for alpaca B44's 2nd to 4th immune bleed responses to the biotinylated mutated survivin-derived peptide (1B) and the biotinylated wild-type survivin-derived peptide (1C).

FIG. 35 depicts the ELISA data for alpaca A100's 2nd to 4th immune bleed responses to the biotinylated mutated survivin-derived peptide (1B) and the biotinylated wild-type survivin-derived peptide (1C).

FIG. 36 depicts the ELISA data for alpaca B44's 3rd to 5th immune bleeds using the biotinylated mutated survivin-derived peptide (1B).

FIG. 37 depicts the ELISA data for alpaca A100's 3rd to 5th immune bleeds using the biotinylated mutated survivin-derived peptide (1B).

FIG. 38 depicts the panning process of the actively immunized single-domain antibody (sdAb) library against the survivin-derived antigen. The figure outlines two rounds of biopanning using biotinylated peptides: round 1 with the wild-type peptide (1C) and round 2 with the mutated peptide (1B).

FIG. 39 depicts the ELISA data for pool S1 following the first round of biopanning using the biotinylated mutated survivin-derived peptide (1B). The table shows the binding affinities of selected sdAbs to the peptide, highlighting clones with significant binding.

FIG. 40 depicts the ELISA data for pool S1 following the first round of biopanning using the biotinylated wild-type survivin-derived peptide (1C). The table presents the binding affinities of selected sdAbs to the peptide, indicating specific clones with high affinity.

FIG. 41 depicts the control ELISA data for pool S1 using an isotype biotin peptide. This serves as a benchmark to assess the specificity of the antibody-antigen interaction, highlighting non-specific binding levels.

FIG. 42 depicts the nucleotide and corresponding amino acid sequences of the complementarity-determining regions (CDRs) of the heavy chain variable domains (VHH) from the sdAbs selected after the first round of biopanning.

FIG. 43 depicts the ELISA data for pool S1 following the second round of biopanning using the biotinylated mutated survivin-derived peptide (1B). The table shows the binding affinities of a larger number of selected sdAbs to the peptide, indicating a significant increase in binding activity compared to the first round.

FIG. 44 depicts the ELISA data for pool S3 following the second round of biopanning using the wildtype peptide 1C. The data indicates a significant binding activity, validating the increased affinity of selected clones for the wildtype peptide.

FIG. 45 depicts the control data for pool S3 using an isotype biotin peptide following the second round of biopanning. This serves as a benchmark to assess the specificity of the antibody-antigen interaction, highlighting non-specific binding levels.

FIG. 46 depicts the nucleotide and corresponding amino acid sequences of the complementarity-determining regions (CDRs) of the heavy chain variable domains (VHH) from the selected sdAbs after the second round of biopanning. The figure presents a comparative analysis of 31 unique clones.

FIG. 47 depicts a phylogenetic tree illustrating the relationship between the selected sdAb clones based on their sequence alignment. The tree identifies Clone 1 (AHP37746), Clone 7 (AHP37725), and Clone 9 (AHP37722) as lead candidates based on their unique sequences and high binding affinity.

FIG. 48 depicts the ELISA results for the binding of selected sdAb clones (Clone 1, Clone 7, and Clone 9) to wild-type survivin (WT-SVN).

FIG. 49 depicts the ELISA results for the mutant M57-SVN form of survivin, highlighting the binding affinity of sdAb clones 1, 7, and 9.

FIG. 50 depicts the flow cytometry analysis and fluorescence microscopy of sdAb clones binding to non-permeabilized HeLa cells. The histogram plots show binding intensities for clones AHP37746, AHP37725, and AHP37722, while the fluorescence images visualize the binding of clone AHP37746 to cell surfaces.

FIG. 51 depicts the flow cytometry analysis of sdAb clones (Clones 1, 7, and 9) binding to non-permeabilized A1207 cells. The histograms demonstrate the differential binding intensities of each clone.

FIG. 52 depicts fluorescence microscopy images of sdAb clones (Clones 1, 7, and 9) binding to the surface of A1207 cells. The images show the localization of the sdAbs, tagged with fluorescent markers, on the cell surface, confirming the binding specificity.

FIG. 53 depicts the ELISA results showing the half maximal effective concentration (EC50) values for the sdAb clones binding to a biotinylated mutated peptide of survivin.

FIG. 54 depicts the ELISA results showing the EC50 values for the sdAb clones binding to a biotinylated wildtype peptide of survivin.

FIG. 55 depicts the SDS-PAGE analysis of the sdAb clones under reducing and non-reducing conditions. The lanes show the expected molecular weights of the sdAb clones, indicating high purity (>99%) and confirming the monomeric nature of the antibodies.

### DETAILED DESCRIPTION

The present disclosure provides antibodies and antibody fragments, including single variable domains (VHHs) that bind human survivin, as well as nucleic acids, vectors, cells, pharmaceutical compositions, kits, and methods relating to the same.

Several aspects and embodiments of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure.

Any references in the description or in the claims to methods of treatment refer to the compounds, compositions, pharmaceutical compositions and medicaments for use in a method of treatment of the human (or animal) body by therapy (or for diagnosis).

Any references in the description or in the claims to methods of treatment refer to the use of the compounds, compositions, pharmaceutical compositions for the manufacture of a medicament for the treatment of the human (or animal) body by therapy (or for diagnosis).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

### Definitions

The terminology used herein is for the purpose of describing particular aspects or embodiments only and is not intended to be limiting. As used herein, the indefinite articles “a”, “an” and “the” should be understood to include plural reference unless the context clearly indicates otherwise.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

Unless otherwise stated, any numerical value, such as a concentration or a concentration range described herein, are to be understood as being modified in all instances by the term “about” or “approximately”. Thus, a numerical value typically includes ±10% of the recited value. For example, a dosage of 10 mg includes 9 mg to 11 mg. As used herein, the use of a numerical range expressly includes all possible subranges, all individual numerical values within that range, including integers within such ranges and fractions of the values unless the context clearly indicates otherwise.

The term “antibody” includes monoclonal antibodies (including full length 4-chain antibodies or full length heavy-chain only antibodies which have an immunoglobulin Fc region), antibody compositions with polyepitopic specificity, multispecific antibodies (e.g., bispecific antibodies, diabodies, and single-chain molecules), as well as antibody fragments (e.g., Fab, F(ab′)2, and Fv). The term “immunoglobulin” (Ig) is used interchangeably with “antibody” herein. Antibodies contemplated herein include single-domain antibodies (“sdAbs”), such as heavy chain only antibodies. The terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotype (anti-Id) antibodies (including, e.g., anti-Id antibodies to antigen specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Antibody light chains of any vertebrate species may be assigned to one of two clearly distinct types, namely kappa (κ) and lambda (λ), based on the amino acid sequences of their constant domains. The term “antibody” can also encompass recombinantly expressed antigen binding proteins and antigen binding synthetic peptides. The term “antibody” also includes immunoglobulins produced in vivo, as well as those produced in vitro, such as, for example, by a hybridoma.

“Complementarity determining regions” (CDR) are antibody regions that bind an antigen. CDRs may be defined using various delineations such as Kabat (Wu et al. J Exp Med 132: 211-50, 1970) (Kabat et al., Sequences of Proteins of Immunological Interest, 5th Ed. Public Health Service, National Institutes of Health, Bethesda, Md., 1991), Chothia (Chothia et al. J Mol Biol 196: 901-17, 1987), IMGT (Lefranc et al. Dev Comp Immunol 27: 55-77, 2003) and AbM (Martin and Thornton J Bmol Biol 263: 800-15, 1996). The correspondence between the various delineations and variable region numbering are described (see e.g., Lefranc et al. Dev Comp Immunol 27: 55-77, 2003; Honegger and Pluckthun, J Mol Biol 309:657-70, 2001; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification. Correspondence between the numbering system, including, for example, the Kabat numbering and the IMGT unique numbering system, is well known to one skilled in the art (see, e.g., Kabat; Chothia; Martin; Lefranc et al.).

TABLE 1
Kabat, IMGT, AbM, and Chothia numbering systems.

| | IMGT | Kabat | AbM | Chothia |
|---|---|---|---|---|
| V_{H} CDR1 | 27-38 | 31-35 | 26-35 | 26-32 |
| V_{H} CDR2 | 56-65 | 50-65 | 50-58 | 53-55 |
| V_{H} CDR3 | 105-117 | 95-102 | 95-102 | 96-101 |
| V_{L} CDR1 | 27-38 | 24-34 | 24-34 | 26-32 |
| V_{L} CDR2 | 56-65 | 50-56 | 50-56 | 50-52 |
| V_{L} CDR3 | 105-117 | 89-97 | 89-97 | 91-96 |

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “VHHs”. Some VHHs may also be known as “Nanobodies”. A camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

The terms “antigen binding fragment”, “antigen binding domain”, “antibody fragment”, “fragment of an antibody”, “functional fragment of an antibody”, and “antigen-binding portion” are used interchangeably herein to mean one or more fragments or portions of an antibody that retain the ability to specifically bind to an antigen (see, generally, Holliger et al., Nat. Biotech., 23(9): 1 126-1129 (2005)). Antibody fragments can be fragments comprising at least one antibody-antigen binding site. Antibody fragments can, for example, exhibit specific binding to survivin or fragments thereof comprising the motif DLAQCFFCFKELEGW (SEQ ID NO: 204). The antibody fragment may comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CHI domains; (ii) a F(ab′)2 fragment, which is a bivalent fragment comprising two Fab fragments linked by a disulfide bridge at the hinge region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol, 16: 778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Patent Application Publication 2009/0093024 A1. Antigen binding fragments may be synthetic, enzymatically obtainable or genetically engineered polypeptides and include portions of an immunoglobulin that bind an antigen, such as the VH, the VL, the VH and the VL, Fab, Fab′, F(ab′)₂, Fd and Fv fragments, domain antibodies (dAb) consisting of one VH domain or one VL domain, shark variable IgNAR domains, VH domains modified to function without a corresponding VL domain ns, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins.

The term “variable” refers to the fact that certain segments of the variable domains differ extensively in sequence among antibodies. The V domain (i.e., variable domain) mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a 3-sheet configuration, connected by three HVRs, which form loops connecting, and in some cases forming part of, the 3-sheet structure. The HVRs in each chain are held together in close proximity by the FR regions and contribute to the formation of the antigen binding site of antibodies (with the HVRs from the other chain, if the antibody is not a sdAb or HCAb) (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. Heavy-chain only antibodies from the Camelid species have a single heavy chain variable region, which is referred to as “VHH” domain. VHH is thus a special type of variable region.

The term “specificity” refers to selective recognition of an antigen binding protein (such as a VHH) for a particular epitope of an antigen. Natural antibodies, for example, are monospecific. The term “multispecific” refers to an antibody that specifically binds at least two distinct antigens or at least two distinct epitopes within the same antigen. Multispecific antibody may bind for example two, three, four or five distinct antigens or distinct epitopes within the same antigen. “Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

The term “survivin peptide” or “survivin peptides” as used herein means fragments of full length survivin and includes variants of the peptides which can generate antibodies that react with the wild type survivin, such as human survivin. The term “anti-survivin antibodies” as used herein means antibodies that are generated in response to survivin or one or more survivin peptides (including variants thereof).

“Cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

“Combination” means that two or more therapeutics are administered to a subject together in a mixture, concurrently as single agents or sequentially as single agents in any order.

“Comprising” is intended to include examples encompassed by the terms “consisting essentially of” and “consisting of”; similarly, the term “consisting essentially of” is intended to include examples encompassed by the term “consisting of” Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In some embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in-between and above 1, e.g., 1.5, 1.6, 1.7, 1.8, etc.) the response (reference response) produced by vehicle, a control composition, or the response in a particular cell lineage. Exemplary functions that can be measured are tumor cell killing.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

By “enhance” or “increase” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in cancer cell death killing ability, among others apparent from the understanding in the art and the description herein. In some embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30, or more times (e.g., 500, 1000 times) (including all integers and decimal points in-between and above 1, e.g., 1.5, 1.6, 1.7, 1.8, etc.) the response produced by vehicle or a control composition. “Enhance” or “enhanced” also refers to enhancement in one or more functions of a test molecule when compared to a control molecule or a combination of test molecules when compared to one or more control molecules. “Enhanced” may be an enhancement of about 10%, 20%, 30%, 40%, 50%, 60%, 70%, 80%, 90%, 100% or more, or a statistically significant enhancement.

The terms “express” and “expression” mean allowing for or causing the information in a gene or DNA sequence to become produced. For example, expression can take the form of producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

“Isolated” refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Mutation” refers to an engineered or naturally occurring alteration in a polypeptide or polynucleotide sequence when compared to a reference sequence. The alteration may be a substitution, insertion or deletion of one or more amino acids or polynucleotides.

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid; these terms may also refer to the nucleic acid sequence including the portion coding for any amino acids added as an artifact of cloning, including any amino acids coded for by linkers.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

“Pharmaceutical composition” refers to composition that comprises an active ingredient and a pharmaceutically acceptable carrier.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

“Pharmaceutically acceptable carrier” or “excipient” refers to an ingredient in a pharmaceutical composition, other than the active ingredient, which is nontoxic to a subject.

The term “protein” or “polypeptide” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

“Recombinant” refers to DNA, antibodies and other proteins that are prepared, expressed, created or isolated by recombinant means when segments from different sources are joined to produce recombinant DNA, antibodies or proteins.

“Relapsed” refers to a cancer that responded to treatment but then returns.

As used herein, the terms “specifically binds”, “specifically recognizes”, or “specific for” refer to measurable and reproducible interactions such as binding between a target and an antigen binding protein (such as a VHH), which is determinative of the presence of the target in the presence of a heterogeneous population of molecules including biological molecules.

As used herein, the term “subject” refers to an animal. The terms “subject” and “patient” may be used interchangeably herein in reference to a subject. As such, a “subject” includes a human that is being treated for a disease, or prevention of a disease, as a patient. The methods described herein may be used to treat an animal subject belonging to any classification. Examples of such animals include mammals. Mammals, include, but are not limited to, mammals of the order Rodentia, such as mice and hamsters, and mammals of the order Logomorpha, such as rabbits. The mammals may be of the order Carnivora, including felines (cats) and canines (dogs). The mammals may be of the order Artiodactyla, including bovines (cows) and swines (pigs) or of the order Perssodactyla, including equines (horses). The mammals may be of the order Primates, Ceboids, or Simoids (monkeys) or of the order Anthropoids (humans and apes). Additionally, the mammals may be of the order Artiodactyla, specifically the family Camelidae, which includes species such as camels, llamas, and alpacas. In some embodiments, the mammal is a human.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

The terms “treat” or “treatment” refer to therapeutic treatment wherein the object is to slow down or lessen an undesired physiological change or disease, or provide a beneficial or desired clinical outcome during treatment. Beneficial or desired clinical outcomes include alleviation of symptoms, diminishment of extent of disease, stabilization (i.e., a cessation in the worsening) of the state of disease, delay or slowing of disease progression, amelioration or palliation of the disease state, and/or remission (whether partial or total and whether detectable or undetectable). “Treatment” can also mean prolonging survival as compared to expected survival if a subject was not receiving treatment. Those in need of treatment include those subjects already with the undesired physiological change or disease as well as those subjects prone to having the physiological change or disease. Treatment may involve a treatment agent, also referred to herein as a “medicament” or “medication,” that may be intended to help achieve the beneficial or desired clinical outcome of interest by its action. Treatment agents or medicaments may be administered to a subject by many routes, including at least intravenous and oral routes. The term “intravenous,” in connection to the administration of treatment agents or medicaments, refers to the administration of said treatment agents or medicaments within one or more veins. The term “oral,” in connection to the administration of treatment agents or medicaments, refers to the administration of said treatment agents or medicaments via an oral passage such as the mouth.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

When referring to a dosage amount, “μg/kg” or “mg/kg” refers to the amount of an active agent, such as a bispecific antibody or antibody, in microgram (μg) or milligram (mg) administered to a subject per kilogram (kg) body weight of the subject.

Additionally, throughout this disclosure, various aspects and embodiments of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

The numbering of amino acid residues in the antibody constant region throughout the specification is according to the EU index as described in Kabat et al., Sequences of Proteins of Immunological Interest, 5^{th} Ed. Public Health Service, National Institutes of Health, Bethesda, MD. (1991), unless otherwise explicitly stated. Antibody constant chain numbering can be found for example at ImMunoGeneTics website, at IMGT Web resources at IMGT Scientific charts.

Conventional one and three-letter amino acid codes are used herein as shown in Table 2.

TABLE 2
Amino acid abbreviations.

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### Compositions

This disclosure provides isolated antibodies and fragments thereof, isolated nucleic acid molecules encoding antibodies or fragments thereof, cells producing antibodies or fragments thereof, vectors or cells comprising nucleic acids encoding antibodies or fragments thereof, compositions comprising any of the foregoing, methods of making any of the foregoing, and methods of using the antibodies and fragments thereof, or nucleic acid molecules in the treatment of autoimmune diseases and cancers involving survivin.

In one aspect, this disclosure provides compositions comprising antibodies or fragments thereof, including camelid antibodies, single domain antibodies, human antibodies, humanized antibodies, or chimeric antibodies, which are reactive against one or more epitopes of survivin. Examples of suitable survivin epitopes or variants thereof are provided in U.S. Pat. Nos. 7,943,138, 8,580,269, and 10,738,129, the disclosures of which are incorporated herein by reference. The compositions of the present disclosure comprise antibodies generated in response to administering a peptide that is identical to a sequence within human survivin or is a variant thereof (such as at least 95% identical). For example, antibodies may be generated in and isolated from an individual following administration of a peptide that is variant of the following portion of survivin sequence ENEPDLAQCFFCFKELEGWEPDD (SEQ ID NO: 201). The variant can be ENEPDLAQMFFCFKELEGWEPDD (SEQ ID NO: 202, a C to M change at position 9 of SEQ ID NO: 201). The peptides administered can be from 9 to 23 (including all integers therebetween) contiguous amino acids of SEQ ID NO: 202, wherein the peptide comprises the core sequence of QMFFCF (SEQ ID NO: 203). Exemplary survivin peptides include DLAQMFFCFKELEGW (SEQ ID NO: 204), AQMFFCFKEL (SEQ ID NO: 205), and QMFFCFKEL (SEQ ID NO: 206). The isolated antibodies or fragments thereof may be used without modifications, or they may be engineered, such as, for example, to generate chimeric or humanized antibodies or various fragments as described therein. In one embodiment, humanized antibodies or fragments thereof are generated that are reactive against the peptide DLAQMFFCFKELEGW (SEQ ID NO: 204).

The antibodies, polypeptides, and proteins of embodiments of the disclosure (including functional portions and functional variants) can be subject to post-translational modifications. They can be glycosylated, esterified, N-acylated, amidated, carboxylated, phosphorylated, esterified, cyclized via, e.g., a disulfide bridge, or converted into an acid addition salt. In some embodiments, they are dimerized or polymerized, or conjugated.

In one embodiment, this disclosure provides isolated antibodies. By the term “isolated” it is meant that the antibody or the fragment thereof, is separated and/or recovered from its natural environment. The isolation of the antibody from its natural environment can be such that the antibody can be used without interference from other active agents (such as other proteins) that normally are present in its natural environment.

In one embodiment, this disclosure provides generating and isolating single domain antibodies or nanobodies produced by camelids in response to introducing survivin or survivin peptides into the camelids. The nanobodies are typically heavy chain antibodies and thus contain heavy chain homodimers and do not contain antibody light chains. These antibodies typically comprise a single variable domain and two constant domains (CH2 and CH3).

In some embodiments, there are antibodies, or an antigen-binding fragment thereof, wherein the antibody or antigen-binding fragment thereof comprises a heavy chain variable domain (VHH) comprising a complementarity-determining region (CDR) 1, a CDR2, and a CDR3. In some embodiments, there are compositions comprising survivin antigen binding domains comprising at least one anti-survivin binding moiety. In some embodiments, the at least one anti-survivin binding moiety is camelid, chimeric, human, or humanized. In some embodiments, one or more of the binding moieties are antigen binding fragments. In some embodiments, one or more of the binding moieties comprise single-domain antibodies. In some embodiments, one or more of the binding moieties comprise a VHH.

In some embodiments, the antigen-binding fragment comprises a first complementarity determining region (CDR1) comprising the amino acid sequence of SEQ ID NO: 1. In some embodiments, the antigen-binding fragment comprises a second complementarity determining region (CDR2) comprising the amino acid sequence of SEQ ID NO: 2. In some embodiments, the antigen-binding fragment comprises a third complementarity determining region (CDR3) comprising the amino acid sequence of SEQ ID NO: 3.

In some embodiments, the antigen-binding fragment comprises a first complementarity determining region (CDR1) comprising the amino acid sequence of SEQ ID NO: 5. In some embodiments, the antigen-binding fragment comprises a second complementarity determining region (CDR2) comprising the amino acid sequence of SEQ ID NO: 6. In some embodiments, the antigen-binding fragment comprises a third complementarity determining region (CDR3) comprising the amino acid sequence of SEQ ID NO: 7.

In some embodiments, the antigen-binding fragment comprises a first complementarity determining region (CDR1) comprising the amino acid sequence of SEQ ID NO: 9. In some embodiments, the antigen-binding fragment comprises a second complementarity determining region (CDR2) comprising the amino acid sequence of SEQ ID NO: 2. In some embodiments, the antigen-binding fragment comprises a third complementarity determining region (CDR3) comprising the amino acid sequence of SEQ ID NO: 10.

In some embodiments, the antigen-binding fragment comprises a first complementarity determining region (CDR1) comprising the amino acid sequence of SEQ ID NO: 9. In some embodiments, the antigen-binding fragment comprises a second complementarity determining region (CDR2) comprising the amino acid sequence of SEQ ID NO: 12. In some embodiments, the antigen-binding fragment comprises a third complementarity determining region (CDR3) comprising the amino acid sequence of SEQ ID NO: 13.

In some embodiments, the antigen-binding fragment comprises a first complementarity determining region (CDR1) comprising the amino acid sequence of SEQ ID NO: 15. In some embodiments, the antigen-binding fragment comprises a second complementarity determining region (CDR2) comprising the amino acid sequence of SEQ ID NO: 12. In some embodiments, the antigen-binding fragment comprises a third complementarity determining region (CDR3) comprising the amino acid sequence of SEQ ID NO: 13.

In some embodiments, the antigen-binding fragment comprises a first complementarity determining region (CDR1) comprising the amino acid sequence of SEQ ID NO: 1. In some embodiments, the antigen-binding fragment comprises a second complementarity determining region (CDR2) comprising the amino acid sequence of SEQ ID NO: 17. In some embodiments, the antigen-binding fragment comprises a third complementarity determining region (CDR3) comprising the amino acid sequence of SEQ ID NO: 7.

In some embodiments, the antigen-binding fragment comprises a first complementarity determining region (CDR1) comprising the amino acid sequence of SEQ ID NO: 1. In some embodiments, the antigen-binding fragment comprises a second complementarity determining region (CDR2) comprising the amino acid sequence of SEQ ID NO: 6. In some embodiments, the antigen-binding fragment comprises a third complementarity determining region (CDR3) comprising the amino acid sequence of SEQ ID NO: 7.

In some embodiments, the antigen-binding fragment comprises a first complementarity determining region (CDR1) comprising the amino acid sequence of SEQ ID NO: 1. In some embodiments, the antigen-binding fragment comprises a second complementarity determining region (CDR2) comprising the amino acid sequence of SEQ ID NO: 20. In some embodiments, the antigen-binding fragment comprises a third complementarity determining region (CDR3) comprising the amino acid sequence of SEQ ID NO: 21.

In some embodiments, the antigen-binding fragment comprises a first complementarity determining region (CDR1) comprising the amino acid sequence of SEQ ID NO: 23. In some embodiments, the antigen-binding fragment comprises a second complementarity determining region (CDR2) comprising the amino acid sequence of SEQ ID NO: 6. In some embodiments, the antigen-binding fragment comprises a third complementarity determining region (CDR3) comprising the amino acid sequence of SEQ ID NO: 24.

In some embodiments, the antigen-binding fragment comprises a first complementarity determining region (CDR1) comprising the amino acid sequence of SEQ ID NO: 9. In some embodiments, the antigen-binding fragment comprises a second complementarity determining region (CDR2) comprising the amino acid sequence of SEQ ID NO: 26. In some embodiments, the antigen-binding fragment comprises a third complementarity determining region (CDR3) comprising the amino acid sequence of SEQ ID NO: 27.

In some embodiments, the antigen-binding fragment comprises a first complementarity determining region (CDR1) comprising the amino acid sequence of SEQ ID NO: 9. In some embodiments, the antigen-binding fragment comprises a second complementarity determining region (CDR2) comprising the amino acid sequence of SEQ ID NO: 6. In some embodiments, the antigen-binding fragment comprises a third complementarity determining region (CDR3) comprising the amino acid sequence of SEQ ID NO: 29.

In some embodiments, the antigen-binding fragment comprises a first complementarity determining region (CDR1) comprising the amino acid sequence of SEQ ID NO: 31. In some embodiments, the antigen-binding fragment comprises a second complementarity determining region (CDR2) comprising the amino acid sequence of SEQ ID NO: 32. In some embodiments, the antigen-binding fragment comprises a third complementarity determining region (CDR3) comprising the amino acid sequence of SEQ ID NO: 33.

In some embodiments, the antigen-binding fragment comprises a first complementarity determining region (CDR1) comprising the amino acid sequence of SEQ ID NO: 35. In some embodiments, the antigen-binding fragment comprises a second complementarity determining region (CDR2) comprising the amino acid sequence of SEQ ID NO: 36. In some embodiments, the antigen-binding fragment comprises a third complementarity determining region (CDR3) comprising the amino acid sequence of SEQ ID NO: 37.

In some embodiments, the antigen-binding fragment comprises a first complementarity determining region (CDR1) comprising the amino acid sequence of SEQ ID NO: 39. In some embodiments, the antigen-binding fragment comprises a second complementarity determining region (CDR2) comprising the amino acid sequence of SEQ ID NO: 40. In some embodiments, the antigen-binding fragment comprises a third complementarity determining region (CDR3) comprising the amino acid sequence of SEQ ID NO: 37.

In some embodiments, the antigen-binding fragment comprises a first complementarity determining region (CDR1) comprising the amino acid sequence of SEQ ID NO: 35. In some embodiments, the antigen-binding fragment comprises a second complementarity determining region (CDR2) comprising the amino acid sequence of SEQ ID NO: 42. In some embodiments, the antigen-binding fragment comprises a third complementarity determining region (CDR3) comprising the amino acid sequence of SEQ ID NO: 37.

In some embodiments, the antigen-binding fragment comprises a first complementarity determining region (CDR1) comprising the amino acid sequence of SEQ ID NO: 39. In some embodiments, the antigen-binding fragment comprises a second complementarity determining region (CDR2) comprising the amino acid sequence of SEQ ID NO: 42. In some embodiments, the antigen-binding fragment comprises a third complementarity determining region (CDR3) comprising the amino acid sequence of SEQ ID NO: 37.

In some embodiments, the antigen-binding fragment comprises a first complementarity determining region (CDR1) comprising the amino acid sequence of SEQ ID NO: 39. In some embodiments, the antigen-binding fragment comprises a second complementarity determining region (CDR2) comprising the amino acid sequence of SEQ ID NO: 40. In some embodiments, the antigen-binding fragment comprises a third complementarity determining region (CDR3) comprising the amino acid sequence of SEQ ID NO: 45.

In some embodiments, the antigen-binding fragment comprises a first complementarity determining region (CDR1) comprising the amino acid sequence of SEQ ID NO: 47. In some embodiments, the antigen-binding fragment comprises a second complementarity determining region (CDR2) comprising the amino acid sequence of SEQ ID NO: 42. In some embodiments, the antigen-binding fragment comprises a third complementarity determining region (CDR3) comprising the amino acid sequence of SEQ ID NO: 45.

In some embodiments, the antigen-binding fragment comprises a first complementarity determining region (CDR1) comprising the amino acid sequence of SEQ ID NO: 49. In some embodiments, the antigen-binding fragment comprises a second complementarity determining region (CDR2) comprising the amino acid sequence of SEQ ID NO: 42. In some embodiments, the antigen-binding fragment comprises a third complementarity determining region (CDR3) comprising the amino acid sequence of SEQ ID NO: 45.

In some embodiments, the antigen-binding fragment comprises a first complementarity determining region (CDR1) comprising the amino acid sequence of SEQ ID NO: 51. In some embodiments, the antigen-binding fragment comprises a second complementarity determining region (CDR2) comprising the amino acid sequence of SEQ ID NO: 42. In some embodiments, the antigen-binding fragment comprises a third complementarity determining region (CDR3) comprising the amino acid sequence of SEQ ID NO: 45.

In some embodiments, the antigen-binding fragment comprises a first complementarity determining region (CDR1) comprising the amino acid sequence of SEQ ID NO: 53. In some embodiments, the antigen-binding fragment comprises a second complementarity determining region (CDR2) comprising the amino acid sequence of SEQ ID NO: 54. In some embodiments, the antigen-binding fragment comprises a third complementarity determining region (CDR3) comprising the amino acid sequence of SEQ ID NO: 55.

In some embodiments, the antigen-binding fragment comprises a first complementarity determining region (CDR1) comprising the amino acid sequence of SEQ ID NO: 57. In some embodiments, the antigen-binding fragment comprises a second complementarity determining region (CDR2) comprising the amino acid sequence of SEQ ID NO: 54. In some embodiments, the antigen-binding fragment comprises a third complementarity determining region (CDR3) comprising the amino acid sequence of SEQ ID NO: 55.

In some embodiments, the antigen-binding fragment comprises a first complementarity determining region (CDR1) comprising the amino acid sequence of SEQ ID NO: 9. In some embodiments, the antigen-binding fragment comprises a second complementarity determining region (CDR2) comprising the amino acid sequence of SEQ ID NO: 54. In some embodiments, the antigen-binding fragment comprises a third complementarity determining region (CDR3) comprising the amino acid sequence of SEQ ID NO: 55.

In some embodiments, the antigen-binding fragment comprises a first complementarity determining region (CDR1) comprising the amino acid sequence of SEQ ID NO: 60. In some embodiments, the antigen-binding fragment comprises a second complementarity determining region (CDR2) comprising the amino acid sequence of SEQ ID NO: 42. In some embodiments, the antigen-binding fragment comprises a third complementarity determining region (CDR3) comprising the amino acid sequence of SEQ ID NO: 55.

In some embodiments, the antigen-binding fragment comprises a first complementarity determining region (CDR1) comprising the amino acid sequence of SEQ ID NO: 62. In some embodiments, the antigen-binding fragment comprises a second complementarity determining region (CDR2) comprising the amino acid sequence of SEQ ID NO: 42. In some embodiments, the antigen-binding fragment comprises a third complementarity determining region (CDR3) comprising the amino acid sequence of SEQ ID NO: 55.

In some embodiments, the antigen-binding fragment comprises a first complementarity determining region (CDR1) comprising the amino acid sequence of SEQ ID NO: 64. In some embodiments, the antigen-binding fragment comprises a second complementarity determining region (CDR2) comprising the amino acid sequence of SEQ ID NO: 42. In some embodiments, the antigen-binding fragment comprises a third complementarity determining region (CDR3) comprising the amino acid sequence of SEQ ID NO: 55.

In some embodiments, the antigen-binding fragment comprises a first complementarity determining region (CDR1) comprising the amino acid sequence of SEQ ID NO: 47. In some embodiments, the antigen-binding fragment comprises a second complementarity determining region (CDR2) comprising the amino acid sequence of SEQ ID NO: 42. In some embodiments, the antigen-binding fragment comprises a third complementarity determining region (CDR3) comprising the amino acid sequence of SEQ ID NO: 55.

In some embodiments, the antigen-binding fragment comprises a first complementarity determining region (CDR1) comprising the amino acid sequence of SEQ ID NO: 35. In some embodiments, the antigen-binding fragment comprises a second complementarity determining region (CDR2) comprising the amino acid sequence of SEQ ID NO: 42. In some embodiments, the antigen-binding fragment comprises a third complementarity determining region (CDR3) comprising the amino acid sequence of SEQ ID NO: 55.

In some embodiments, the antigen-binding fragment comprises a first complementarity determining region (CDR1) comprising the amino acid sequence of SEQ ID NO: 68. In some embodiments, the antigen-binding fragment comprises a second complementarity determining region (CDR2) comprising the amino acid sequence of SEQ ID NO: 42. In some embodiments, the antigen-binding fragment comprises a third complementarity determining region (CDR3) comprising the amino acid sequence of SEQ ID NO: 55.

In some embodiments, the antigen-binding fragment comprises a first complementarity determining region (CDR1) comprising the amino acid sequence of SEQ ID NO: 49. In some embodiments, the antigen-binding fragment comprises a second complementarity determining region (CDR2) comprising the amino acid sequence of SEQ ID NO: 42. In some embodiments, the antigen-binding fragment comprises a third complementarity determining region (CDR3) comprising the amino acid sequence of SEQ ID NO: 55.

In some embodiments, the antigen-binding fragment comprises a first complementarity determining region (CDR1) comprising the amino acid sequence of SEQ ID NO: 1. In some embodiments, the antigen-binding fragment comprises a second complementarity determining region (CDR2) comprising the amino acid sequence of SEQ ID NO: 20. In some embodiments, the antigen-binding fragment comprises a third complementarity determining region (CDR3) comprising the amino acid sequence of SEQ ID NO: 71.

In some embodiments, the antigen-binding fragment comprises a first complementarity determining region (CDR1) comprising the amino acid sequence of SEQ ID NO: 73. In some embodiments, the antigen-binding fragment comprises a second complementarity determining region (CDR2) comprising the amino acid sequence of SEQ ID NO: 74. In some embodiments, the antigen-binding fragment comprises a third complementarity determining region (CDR3) comprising the amino acid sequence of SEQ ID NO: 75.

In some embodiments, the antigen-binding fragment comprises a first complementarity determining region (CDR1) comprising the amino acid sequence of SEQ ID NO: 77. In some embodiments, the antigen-binding fragment comprises a second complementarity determining region (CDR2) comprising the amino acid sequence of SEQ ID NO: 78. In some embodiments, the antigen-binding fragment comprises a third complementarity determining region (CDR3) comprising the amino acid sequence of SEQ ID NO: 79.

In some embodiments, the antigen-binding fragment comprises a first complementarity determining region (CDR1) comprising the amino acid sequence of SEQ ID NO: 81. In some embodiments, the antigen-binding fragment comprises a second complementarity determining region (CDR2) comprising the amino acid sequence of SEQ ID NO: 82. In some embodiments, the antigen-binding fragment comprises a third complementarity determining region (CDR3) comprising the amino acid sequence of SEQ ID NO: 83.

In some embodiments, the antigen-binding fragment comprises a first complementarity determining region (CDR1) comprising the amino acid sequence of SEQ ID NO: 31. In some embodiments, the antigen-binding fragment comprises a second complementarity determining region (CDR2) comprising the amino acid sequence of SEQ ID NO: 85. In some embodiments, the antigen-binding fragment comprises a third complementarity determining region (CDR3) comprising the amino acid sequence of SEQ ID NO: 86.

In some embodiments, the antigen-binding fragment comprises a first complementarity determining region (CDR1) comprising the amino acid sequence of SEQ ID NO: 88. In some embodiments, the antigen-binding fragment comprises a second complementarity determining region (CDR2) comprising the amino acid sequence of SEQ ID NO: 78. In some embodiments, the antigen-binding fragment comprises a third complementarity determining region (CDR3) comprising the amino acid sequence of SEQ ID NO: 89.

In some embodiments, the antigen-binding fragment comprises a first complementarity determining region (CDR1) comprising the amino acid sequence of SEQ ID NO: 91. In some embodiments, the antigen-binding fragment comprises a second complementarity determining region (CDR2) comprising the amino acid sequence of SEQ ID NO: 92. In some embodiments, the antigen-binding fragment comprises a third complementarity determining region (CDR3) comprising the amino acid sequence of SEQ ID NO: 93.

In some embodiments, the antigen-binding fragment comprises a first complementarity determining region (CDR1) comprising the amino acid sequence of SEQ ID NO: 95. In some embodiments, the antigen-binding fragment comprises a second complementarity determining region (CDR2) comprising the amino acid sequence of SEQ ID NO: 96. In some embodiments, the antigen-binding fragment comprises a third complementarity determining region (CDR3) comprising the amino acid sequence of SEQ ID NO: 97.

In some embodiments, the antigen-binding fragment comprises a first complementarity determining region (CDR1) comprising the amino acid sequence of SEQ ID NO: 99. In some embodiments, the antigen-binding fragment comprises a second complementarity determining region (CDR2) comprising the amino acid sequence of SEQ ID NO: 100. In some embodiments, the antigen-binding fragment comprises a third complementarity determining region (CDR3) comprising the amino acid sequence of SEQ ID NO: 101.

In some embodiments, the antigen-binding fragment comprises a first complementarity determining region (CDR1) comprising the amino acid sequence of SEQ ID NO: 103. In some embodiments, the antigen-binding fragment comprises a second complementarity determining region (CDR2) comprising the amino acid sequence of SEQ ID NO: 104. In some embodiments, the antigen-binding fragment comprises a third complementarity determining region (CDR3) comprising the amino acid sequence of SEQ ID NO: 105.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 4. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 4, provided that CDR1 comprises the amino acid sequence of SEQ ID NO: 1, CDR2 comprises the amino acid sequence of SEQ ID NO: 2, and CDR3 comprises the amino acid sequence of SEQ ID NO: 3. In some embodiments, the VHH of the antibody, or antigen-binding fragment thereof, comprises an amino acid sequence that is identical to SEQ ID NO: 4.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 8. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 8, provided that CDR1 comprises the amino acid sequence of SEQ ID NO: 5, CDR2 comprises the amino acid sequence of SEQ ID NO: 6, and CDR3 comprises the amino acid sequence of SEQ ID NO: 7. In some embodiments, the VHH of the antibody, or antigen-binding fragment thereof, comprises an amino acid sequence that is identical to SEQ ID NO: 8.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 11. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 11, provided that CDR1 comprises the amino acid sequence of SEQ ID NO: 9, CDR2 comprises the amino acid sequence of SEQ ID NO: 2, and CDR3 comprises the amino acid sequence of SEQ ID NO: 10. In some embodiments, the VHH of the antibody, or antigen-binding fragment thereof, comprises an amino acid sequence that is identical to SEQ ID NO: 11.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 14. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 14, provided that CDR1 comprises the amino acid sequence of SEQ ID NO: 9, CDR2 comprises the amino acid sequence of SEQ ID NO: 12, and CDR3 comprises the amino acid sequence of SEQ ID NO: 13. In some embodiments, the VHH of the antibody, or antigen-binding fragment thereof, comprises an amino acid sequence that is identical to SEQ ID NO: 14.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 16. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 16, provided that CDR1 comprises the amino acid sequence of SEQ ID NO: 15, CDR2 comprises the amino acid sequence of SEQ ID NO: 12, and CDR3 comprises the amino acid sequence of SEQ ID NO: 13. In some embodiments, the VHH of the antibody, or antigen-binding fragment thereof, comprises an amino acid sequence that is identical to SEQ ID NO: 16.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 18. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 18, provided that CDR1 comprises the amino acid sequence of SEQ ID NO: 1, CDR2 comprises the amino acid sequence of SEQ ID NO: 17, and CDR3 comprises the amino acid sequence of SEQ ID NO: 7. In some embodiments, the VHH of the antibody, or antigen-binding fragment thereof, comprises an amino acid sequence that is identical to SEQ ID NO: 18.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 19. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 19, provided that CDR1 comprises the amino acid sequence of SEQ ID NO: 1, CDR2 comprises the amino acid sequence of SEQ ID NO: 6, and CDR3 comprises the amino acid sequence of SEQ ID NO: 7. In some embodiments, the VHH of the antibody, or antigen-binding fragment thereof, comprises an amino acid sequence that is identical to SEQ ID NO: 19.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 22. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 22, provided that CDR1 comprises the amino acid sequence of SEQ ID NO: 1, CDR2 comprises the amino acid sequence of SEQ ID NO: 20, and CDR3 comprises the amino acid sequence of SEQ ID NO: 21. In some embodiments, the VHH of the antibody, or antigen-binding fragment thereof, comprises an amino acid sequence that is identical to SEQ ID NO: 22.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 25. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 25, provided that CDR1 comprises the amino acid sequence of SEQ ID NO: 23, CDR2 comprises the amino acid sequence of SEQ ID NO: 6, and CDR3 comprises the amino acid sequence of SEQ ID NO: 24. In some embodiments, the VHH of the antibody, or antigen-binding fragment thereof, comprises an amino acid sequence that is identical to SEQ ID NO: 25.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 28. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 28, provided that CDR1 comprises the amino acid sequence of SEQ ID NO: 9, CDR2 comprises the amino acid sequence of SEQ ID NO: 26, and CDR3 comprises the amino acid sequence of SEQ ID NO: 27. In some embodiments, the VHH of the antibody, or antigen-binding fragment thereof, comprises an amino acid sequence that is identical to SEQ ID NO: 28.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 30. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 30, provided that CDR1 comprises the amino acid sequence of SEQ ID NO: 9, CDR2 comprises the amino acid sequence of SEQ ID NO: 6, and CDR3 comprises the amino acid sequence of SEQ ID NO: 29. In some embodiments, the VHH of the antibody, or antigen-binding fragment thereof, comprises an amino acid sequence that is identical to SEQ ID NO: 30.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 34. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 34, provided that CDR1 comprises the amino acid sequence of SEQ ID NO: 31, CDR2 comprises the amino acid sequence of SEQ ID NO: 32, and CDR3 comprises the amino acid sequence of SEQ ID NO: 33. In some embodiments, the VHH of the antibody, or antigen-binding fragment thereof, comprises an amino acid sequence that is identical to SEQ ID NO: 34.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 38. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 38, provided that CDR1 comprises the amino acid sequence of SEQ ID NO: 35, CDR2 comprises the amino acid sequence of SEQ ID NO: 36, and CDR3 comprises the amino acid sequence of SEQ ID NO: 37. In some embodiments, the VHH of the antibody, or antigen-binding fragment thereof, comprises an amino acid sequence that is identical to SEQ ID NO: 38.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 41. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 41, provided that CDR1 comprises the amino acid sequence of SEQ ID NO: 39, CDR2 comprises the amino acid sequence of SEQ ID NO: 40, and CDR3 comprises the amino acid sequence of SEQ ID NO: 37. In some embodiments, the VHH of the antibody, or antigen-binding fragment thereof, comprises an amino acid sequence that is identical to SEQ ID NO: 41.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 43. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 43, provided that CDR1 comprises the amino acid sequence of SEQ ID NO: 35, CDR2 comprises the amino acid sequence of SEQ ID NO: 42, and CDR3 comprises the amino acid sequence of SEQ ID NO: 37. In some embodiments, the VHH of the antibody, or antigen-binding fragment thereof, comprises an amino acid sequence that is identical to SEQ ID NO: 43.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 44. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 44, provided that CDR1 comprises the amino acid sequence of SEQ ID NO: 39, CDR2 comprises the amino acid sequence of SEQ ID NO: 42, and CDR3 comprises the amino acid sequence of SEQ ID NO: 37. In some embodiments, the VHH of the antibody, or antigen-binding fragment thereof, comprises an amino acid sequence that is identical to SEQ ID NO: 44.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 46. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 46, provided that CDR1 comprises the amino acid sequence of SEQ ID NO: 39, CDR2 comprises the amino acid sequence of SEQ ID NO: 40, and CDR3 comprises the amino acid sequence of SEQ ID NO: 45. In some embodiments, the VHH of the antibody, or antigen-binding fragment thereof, comprises an amino acid sequence that is identical to SEQ ID NO: 46.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 48. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 48, provided that CDR1 comprises the amino acid sequence of SEQ ID NO: 47, CDR2 comprises the amino acid sequence of SEQ ID NO: 42, and CDR3 comprises the amino acid sequence of SEQ ID NO: 45. In some embodiments, the VHH of the antibody, or antigen-binding fragment thereof, comprises an amino acid sequence that is identical to SEQ ID NO: 48.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 50. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 50, provided that CDR1 comprises the amino acid sequence of SEQ ID NO: 49, CDR2 comprises the amino acid sequence of SEQ ID NO: 42, and CDR3 comprises the amino acid sequence of SEQ ID NO: 45. In some embodiments, the VHH of the antibody, or antigen-binding fragment thereof, comprises an amino acid sequence that is identical to SEQ ID NO: 50.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 52. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 52, provided that CDR1 comprises the amino acid sequence of SEQ ID NO: 51, CDR2 comprises the amino acid sequence of SEQ ID NO: 42, and CDR3 comprises the amino acid sequence of SEQ ID NO: 45. In some embodiments, the VHH of the antibody, or antigen-binding fragment thereof, comprises an amino acid sequence that is identical to SEQ ID NO: 52.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 56. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 56, provided that CDR1 comprises the amino acid sequence of SEQ ID NO: 53, CDR2 comprises the amino acid sequence of SEQ ID NO: 54, and CDR3 comprises the amino acid sequence of SEQ ID NO: 55. In some embodiments, the VHH of the antibody, or antigen-binding fragment thereof, comprises an amino acid sequence that is identical to SEQ ID NO: 56.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 58. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 58, provided that CDR1 comprises the amino acid sequence of SEQ ID NO: 57, CDR2 comprises the amino acid sequence of SEQ ID NO: 54, and CDR3 comprises the amino acid sequence of SEQ ID NO: 55. In some embodiments, the VHH of the antibody, or antigen-binding fragment thereof, comprises an amino acid sequence that is identical to SEQ ID NO: 58.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 59. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 59, provided that CDR1 comprises the amino acid sequence of SEQ ID NO: 9, CDR2 comprises the amino acid sequence of SEQ ID NO: 54, and CDR3 comprises the amino acid sequence of SEQ ID NO: 55. In some embodiments, the VHH of the antibody, or antigen-binding fragment thereof, comprises an amino acid sequence that is identical to SEQ ID NO: 59.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 61. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 61, provided that CDR1 comprises the amino acid sequence of SEQ ID NO: 60, CDR2 comprises the amino acid sequence of SEQ ID NO: 42, and CDR3 comprises the amino acid sequence of SEQ ID NO: 55. In some embodiments, the VHH of the antibody, or antigen-binding fragment thereof, comprises an amino acid sequence that is identical to SEQ ID NO: 61.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 63. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 63, provided that CDR1 comprises the amino acid sequence of SEQ ID NO: 62, CDR2 comprises the amino acid sequence of SEQ ID NO: 42, and CDR3 comprises the amino acid sequence of SEQ ID NO: 55. In some embodiments, the VHH of the antibody, or antigen-binding fragment thereof, comprises an amino acid sequence that is identical to SEQ ID NO: 63.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 65. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 65, provided that CDR1 comprises the amino acid sequence of SEQ ID NO: 64, CDR2 comprises the amino acid sequence of SEQ ID NO: 42, and CDR3 comprises the amino acid sequence of SEQ ID NO: 55. In some embodiments, the VHH of the antibody, or antigen-binding fragment thereof, comprises an amino acid sequence that is identical to SEQ ID NO: 65.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 66. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 66, provided that CDR1 comprises the amino acid sequence of SEQ ID NO: 47, CDR2 comprises the amino acid sequence of SEQ ID NO: 42, and CDR3 comprises the amino acid sequence of SEQ ID NO: 55. In some embodiments, the VHH of the antibody, or antigen-binding fragment thereof, comprises an amino acid sequence that is identical to SEQ ID NO: 66.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 67. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 67, provided that CDR1 comprises the amino acid sequence of SEQ ID NO: 35, CDR2 comprises the amino acid sequence of SEQ ID NO: 42, and CDR3 comprises the amino acid sequence of SEQ ID NO: 55. In some embodiments, the VHH of the antibody, or antigen-binding fragment thereof, comprises an amino acid sequence that is identical to SEQ ID NO: 67.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 69. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 69, provided that CDR1 comprises the amino acid sequence of SEQ ID NO: 68, CDR2 comprises the amino acid sequence of SEQ ID NO: 42, and CDR3 comprises the amino acid sequence of SEQ ID NO: 55. In some embodiments, the VHH of the antibody, or antigen-binding fragment thereof, comprises an amino acid sequence that is identical to SEQ ID NO: 69.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 70. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 70, provided that CDR1 comprises the amino acid sequence of SEQ ID NO: 49, CDR2 comprises the amino acid sequence of SEQ ID NO: 42, and CDR3 comprises the amino acid sequence of SEQ ID NO: 55. In some embodiments, the VHH of the antibody, or antigen-binding fragment thereof, comprises an amino acid sequence that is identical to SEQ ID NO: 70.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 72. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 72, provided that CDR1 comprises the amino acid sequence of SEQ ID NO: 1, CDR2 comprises the amino acid sequence of SEQ ID NO: 20, and CDR3 comprises the amino acid sequence of SEQ ID NO: 71. In some embodiments, the VHH of the antibody, or antigen-binding fragment thereof, comprises an amino acid sequence that is identical to SEQ ID NO: 72.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 76. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 76, provided that CDR1 comprises the amino acid sequence of SEQ ID NO: 73, CDR2 comprises the amino acid sequence of SEQ ID NO: 74, and CDR3 comprises the amino acid sequence of SEQ ID NO: 75. In some embodiments, the VHH of the antibody, or antigen-binding fragment thereof, comprises an amino acid sequence that is identical to SEQ ID NO: 76.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 80. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 80, provided that CDR1 comprises the amino acid sequence of SEQ ID NO: 77, CDR2 comprises the amino acid sequence of SEQ ID NO: 78, and CDR3 comprises the amino acid sequence of SEQ ID NO: 79. In some embodiments, the VHH of the antibody, or antigen-binding fragment thereof, comprises an amino acid sequence that is identical to SEQ ID NO: 80.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 84. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 84, provided that CDR1 comprises the amino acid sequence of SEQ ID NO: 81, CDR2 comprises the amino acid sequence of SEQ ID NO: 82, and CDR3 comprises the amino acid sequence of SEQ ID NO: 83. In some embodiments, the VHH of the antibody, or antigen-binding fragment thereof, comprises an amino acid sequence that is identical to SEQ ID NO: 84.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 87. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 87, provided that CDR1 comprises the amino acid sequence of SEQ ID NO: 31, CDR2 comprises the amino acid sequence of SEQ ID NO: 85, and CDR3 comprises the amino acid sequence of SEQ ID NO: 86. In some embodiments, the VHH of the antibody, or antigen-binding fragment thereof, comprises an amino acid sequence that is identical to SEQ ID NO: 87.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 90. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 90, provided that CDR1 comprises the amino acid sequence of SEQ ID NO: 88, CDR2 comprises the amino acid sequence of SEQ ID NO: 78, and CDR3 comprises the amino acid sequence of SEQ ID NO: 89. In some embodiments, the VHH of the antibody, or antigen-binding fragment thereof, comprises an amino acid sequence that is identical to SEQ ID NO: 90.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 94. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 94, provided that CDR1 comprises the amino acid sequence of SEQ ID NO: 91, CDR2 comprises the amino acid sequence of SEQ ID NO: 92, and CDR3 comprises the amino acid sequence of SEQ ID NO: 93. In some embodiments, the VHH of the antibody, or antigen-binding fragment thereof, comprises an amino acid sequence that is identical to SEQ ID NO: 94.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 98. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 98, provided that CDR1 comprises the amino acid sequence of SEQ ID NO: 95, CDR2 comprises the amino acid sequence of SEQ ID NO: 96, and CDR3 comprises the amino acid sequence of SEQ ID NO: 97. In some embodiments, the VHH of the antibody, or antigen-binding fragment thereof, comprises an amino acid sequence that is identical to SEQ ID NO: 98.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 102. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 102, provided that CDR1 comprises the amino acid sequence of SEQ ID NO: 99, CDR2 comprises the amino acid sequence of SEQ ID NO: 100, and CDR3 comprises the amino acid sequence of SEQ ID NO: 101. In some embodiments, the VHH of the antibody, or antigen-binding fragment thereof, comprises an amino acid sequence that is identical to SEQ ID NO: 102.

In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 106. In some embodiments, the VHH of the antibody, or antigen-binding fragment thereof, comprises an amino acid sequence having at least 90%, 91%, 92%, 93%, 94%, 95%, 96%, 97%, 98%, or 99% sequence identity to SEQ ID NO: 106, provided that CDR1 comprises the amino acid sequence of SEQ ID NO: 103, CDR2 comprises the amino acid sequence of SEQ ID NO: 104, and CDR3 comprises the amino acid sequence of SEQ ID NO: 105. In some embodiments, the VHH of the antibody, or antigen-binding fragment thereof, comprises an amino acid sequence that is identical to SEQ ID NO: 106.

### Nucleic Acids, Vectors, and Host Cells

A “vector” is a replicon, such as plasmid, phage, cosmid, or virus in which another nucleic acid segment may be operably inserted so as to bring about the replication or expression of the segment.

As used herein, the term “host cell” can be any type of cell, e.g., a primary cell, a cell in culture, or a cell from a cell line. In specific embodiments, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome. The terms “expression” and “production” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications.

In some embodiments, the disclosure provides an isolated nucleic acid molecule encodes an antibody, or antigen-binding fragment thereof. In some embodiments, the disclosure provides an expression vector comprising any nucleic acid molecule disclosed herein. In some embodiments, the disclosure provides a host cell comprising any nucleic acid molecule disclosed herein or any expression vector disclosed herein.

In some embodiments, the disclosure provides cells comprising an expression vector or other polynucleotide sequence encoding the antibodies provided herein or survivin binding fragments thereof. Nucleotide sequences encoding the survivin binding fragments thereof can be expressed using any suitable expression vector, many of which are known in the art and/or are commercially available. A vector generally includes nucleic acid sequences, such as origin or replication that enables it to replicate in a host cell. A vector can also include selectable marker genes. Heavy and light chains can be expressed on a single expression vector, such as a plasmid or the heavy and light chains can be expressed on distinct plasmids in the same cell, after which the expressed heavy and light chains can form the antibody architecture. The survivin binding fragments thereof can be isolated and/or purified using conventional techniques, given the benefit of the present disclosure.

### Pharmaceutical Compositions, Dosage Forms, and Administration

In some embodiments, the disclosure provides compositions, e.g., pharmaceutically acceptable compositions, which include a therapeutic compound, polypeptide, protein, or molecule described herein, formulated together with a pharmaceutically acceptable carrier. As used herein, “pharmaceutically acceptable carrier” includes any and all solvents, dispersion media, isotonic and absorption delaying agents, and the like that are physiologically compatible. Suitable carriers include excipients, or stabilizers which are nontoxic to recipients at the dosages and concentrations employed, and include buffers such as acetate, Tris, phosphate, citrate, and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; and/or non-ionic surfactants such as Tween or polyethylene glycol (PEG). The pharmaceutical compositions may comprise other therapeutic agents.

The carrier can be suitable for intravenous, intramuscular, subcutaneous, parenteral, rectal, local, ophthalmic, topical, spinal or epidermal administration (e.g., by injection or infusion). As used herein, the term “carrier” means a diluent, adjuvant, or excipient with which a compound is administered. In some embodiments, pharmaceutical carriers can also be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating, and coloring agents can be used. The carriers can be used in pharmaceutical compositions comprising the therapeutic compounds provided for herein.

Therapeutic compositions typically are sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The compositions and compounds of the embodiments provided herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. The compositions of the present disclosure may be administered by routine methods known in the art. Typical compositions are in the form of injectable or infusible solutions. In some embodiments, the mode of administration is parenteral (e.g., intravenous, subcutaneous, intraperitoneal, intramuscular). In some embodiments, the pharmaceutical composition is an injectable pharmaceutical composition. In some embodiments, the therapeutic molecule is administered by intravenous infusion or injection. In another embodiment, the therapeutic molecule is administered by intramuscular or subcutaneous injection. In another embodiment, the therapeutic molecule is administered locally, e.g., by injection, or topical application, to a target site. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

The pharmaceutical compositions may include a “therapeutically effective amount” or a “prophylactically effective amount” of a therapeutic molecule. A “therapeutically effective amount” refers to an amount effective, at dosages and for periods of time necessary, to achieve the desired therapeutic result. A therapeutically effective amount of a therapeutic molecule may vary according to factors such as the disease state, age, sex, and weight of the individual, and the ability of the therapeutic compound to elicit a desired response in the individual. A therapeutically effective amount is also one in which any toxic or detrimental effects of a therapeutic molecule is outweighed by the therapeutically beneficial effects. A “therapeutically effective dosage” preferably inhibits a measurable parameter, e.g., immune attack at least about 20%, more preferably by at least about 40%, even more preferably by at least about 60%, and still more preferably by at least about 80% relative to untreated subjects. The ability of a compound to inhibit a measurable parameter, e.g., immune attack, can be evaluated in an animal model system predictive of efficacy in glomerular disorders or autoimmune disorders. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

It will be recognized by those of skill in the art that the form and character of the particular dosing regimen employed in the method of the invention will be dictated by the route of administration and other well-known variables, such as the size of the individual and the stage of the disease. Further, the compositions can be provided in the form of unit dosage forms for administration to an individual in need of treatment. Antibodies can be provided in a lyophilized form to be reconstituted prior to administration. The reconstitution medium can be sterile 0.9% saline solution or a suitable physiological buffer or water, or any other solution known in the art for reconstituting proteins prior to administration.

Further, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

### Kits and Articles of Manufacture

Also provided herein are includes kits, e.g., comprising an antibody or an antigen-binding fragment thereof and instructions for the use of the antibody or fragments for killing of particular cell types. The instructions may include directions for using the multispecific antibody or antigen-binding fragment thereof in vitro, in vivo or ex vivo.

Typically, the kit will have a compartment containing the antibody or antigen-binding fragment thereof. The antibody or antigen-binding fragment thereof may be in a lyophilized form, liquid form, or other form amendable to being included in a kit. The kit may also contain additional elements needed to practice the method described on the instructions in the kit, such a sterilized solution for reconstituting a lyophilized powder, additional agents for combining with the multispecific antibody or antigen-binding fragment thereof prior to administering to a patient, and tools that aid in administering the multispecific antibody or antigen-binding fragment thereof to a patient.

The kit can include one or more other elements including: one or more antibodies, which may be in a lyophilized form; optionally reconstitution media; instructions for use; other reagents, e.g., a label, a therapeutic agent, or an agent useful for chelating, or otherwise coupling, a therapeutic molecule to a label or other therapeutic agent, or a radioprotective composition; devices or other materials for preparing the a therapeutic molecule for administration; pharmaceutically acceptable carriers; and devices or other materials for administration to a subject. A kit can comprise a single dose or multiple doses.

In some embodiments, the disclosure provides a kit comprising an antibody, or antibody binding fragment. In some embodiments, the kit comprises an isolated nucleic acid molecule. In some embodiments, the kit comprises an expression vector. In some embodiments, the kit comprises a pharmaceutical composition. In some embodiments, the kit comprises packaging.

The antibodies or fragments thereof can be labeled, such as with enzymatic, fluorescent or radioactive tags or can be conjugated to effector molecules such as, for example, toxins.

In some embodiments, the kit comprises reagents or apparatuses for electroporation of cells.

In some embodiments, the kit comprises artificial antigen presenting cells.

The kits may comprise one or more suitably aliquoted compositions of the present disclosure or reagents to generate compositions of the disclosure. The container means of the kits may include at least one vial, test tube, flask, bottle, syringe, or other container means, into which a component may be placed, and preferably, suitably aliquoted. Where there is more than one component in the kit, the kit also will generally contain a second, third, or other additional container into which the additional components may be separately placed. However, various combinations of components may be comprised in a vial. The kits of the present disclosure also will typically include a means for containing any reagent containers in close confinement for commercial sale. Such containers may include injection or blow molded plastic containers into which the desired vials are retained, for example.

### Methods

The antibodies, polypeptides, and/or proteins of embodiments of the disclosure (including functional portions and functional variants thereof) can be produced by methods known in the art. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, inc., 2000; and Epitope Mapping, ed. Westwood et al., Oxford University Press, Oxford, United Kingdom, 2001. Also, polypeptides and proteins can be recombinantly produced using the nucleic acids described herein using standard recombinant methods. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994. Further, some of the antibodies, polypeptides, and proteins of the disclosure (including functional portions and functional variants thereof) can be isolated and/or purified from a source, such as a plant, a bacterium, an insect, a mammal, etc. Methods of isolation and purification are known in the art. Alternatively, the antibodies, polypeptides, and/or proteins described herein (including functional portions and functional variants thereof) can be commercially synthesized. In this respect, the antibodies, polypeptides, and proteins can be synthetic, recombinant, isolated, and/or purified.

Suitable methods of making antibodies are known in the art. For instance, standard hybridoma methods are described in, e.g., Kohler and Milstein, Eur. J. Immunol., 5, 511-519 (1976), Harlow and Lane (eds.), Antibodies: A Laboratory Manual, CSH Press (1988), and C. A. Janeway et al. (eds.), Immunobiology, 5^{th} Ed., Garland Publishing, New York, N.Y. (2001)). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art.

Phage display can also be used to generate an antibody. In this regard, phage libraries encoding antigen-binding variable (V) domains of antibodies can be generated using standard molecular biology and recombinant DNA techniques (see, e.g., Sambrook et al., supra, and Ausubel et al., supra). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell.

In some embodiments, a method of producing an antibody, or antigen-binding fragment thereof, that binds human survivin is provided, the method comprising (a) growing or culturing a host cell described herein under conditions so that the host cell expresses a polypeptide or polypeptides comprising a heavy chain variable domain (VHH), thereby producing the antibody or the antigen-binding fragment of the antibody; and (b) purifying the antibody, or the antigen-binding fragment thereof.

The present disclosure further related to methods and compositions for use in therapy or the treatment of a disease or condition. In some embodiments, the method is a method of treating a survivin-mediated disorder in a subject in need thereof. In some embodiments, the method comprises administering to the subject an antibody or antibody fragment disclosed herein, or a pharmaceutical composition disclosed herein.

The term “treatment” refers to reduction in one or more symptoms or features associated with the presence of the particular condition being treated. Treatment does not necessarily mean complete remission, nor does it preclude recurrence or relapses. For example, the present disclosure provides a method for reducing the size of a tumor or arresting the growth of a tumor or reducing the rate of growth of a tumor (such as a tumor comprising survivin-expressing cells) or reducing any other symptom that is associated with an individual being afflicted with the tumor-all of which are considered as “treatment” comprising administering to an individual in need of treatment, a therapeutically effective amount of a composition comprising antibodies, or fragments thereof as described herein.

The pharmaceutical compositions and methods containing one or more anti-survivin antibodies are useful to promote catabolism and clearance of pathogenic antibodies, e.g., IgG and IgG autoantibodies in a subject, to reduce the immune response, e.g., to block immune complex-based activation of the immune response in a subject, and to treat immunological conditions or diseases in a subject. In particular, the pharmaceutical compositions and methods are useful to reduce or treat an immune complex-based activation of an acute or chronic immune response. The acute immune response may be activated by a medical condition selected from the group consisting of pemphigus vulgaris, lupus nephritis, myasthenia gravis, Guillain-Barré syndrome, antibody-mediated rejection, catastrophic antiphospholipid antibody syndrome, immune complex-mediated vasculitis, glomerulitis, a channelopathy, neuromyelitis optica, autoimmune hearing loss, idiopathic thrombocytopenia purpura (ITP), autoimmune haemolytic anaemia (AIHA), immune neutropenia, dilated cardiomyopathy, and serum sickness.

In some embodiments, the pharmaceutical compositions and methods are useful to reduce or treat an immune response activated by an autoimmune disease. The autoimmune disease may be selected from the group consisting of alopecia areata, ankylosing spondylitis, antiphospholipid syndrome (e.g., antiphospholipid antibody syndrome), Addison's disease, hemolytic anemia (e.g., warm autoimmune hemolytic anemia), autoimmune hepatitis, hepatitis, Behcets disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, epidermolysis bullosa; fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, membranous nephropathy, Ménière's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis, ulcerative colitis, uveitis, vitiligo, and Wegener's granulomatosis. In some embodiments, the autoimmune disorder is myasthenia gravis.

In some embodiments, the pharmaceutical compositions and methods are useful to reduce or treat a cancer or a tumor, such as a cancer or a tumor that comprise survivin-expressing cells. Such cancers and tumors may be referred to herein as “survivin-expressing cancers” and “survivin-expressing tumors”. Examples of cancers and tumors that can be treated by the present compositions include, but are not limited to, breast cancer, kidney cancer, liver cancer, lung cancer, malignant glioma, melanoma, multiple myeloma, neuroendocrine tumors (NETs), pediatric brain tumors (gliomas), and prostate cancer. In some embodiments, the cancer is stage I, stage II or stage III, and/or stage A or stage B multiple myeloma based on the Durie-Salmon staging system.

The compositions of the invention can be administered prior to, concurrently, or subsequent to other therapies.

### Exemplary Embodiments

The following numerated embodiments are illustrative only and do not limit the scope of the present disclosure.
1. An antibody, or an antigen-binding fragment thereof, wherein the antibody or antigen-binding fragment thereof comprises a heavy chain variable domain (VHH) comprising a complementarity-determining region (CDR) 1, a CDR2, and a CDR3, wherein:
   i. CDR1 comprises the amino acid sequence of SEQ ID NO: 1, CDR2 comprises the amino acid sequence of SEQ ID NO: 2, and CDR3 comprises the amino acid sequence of SEQ ID NO: 3;
   ii. CDR1 comprises the amino acid sequence of SEQ ID NO: 5, CDR2 comprises the amino acid sequence of SEQ ID NO: 6, and CDR3 comprises the amino acid sequence of SEQ ID NO: 7;
   iii. CDR1 comprises the amino acid sequence of SEQ ID NO: 9, CDR2 comprises the amino acid sequence of SEQ ID NO: 2, and CDR3 comprises the amino acid sequence of SEQ ID NO: 10;
   iv. CDR1 comprises the amino acid sequence of SEQ ID NO: 9, CDR2 comprises the amino acid sequence of SEQ ID NO: 12, and CDR3 comprises the amino acid sequence of SEQ ID NO: 13;
   v. CDR1 comprises the amino acid sequence of SEQ ID NO: 15, CDR2 comprises the amino acid sequence of SEQ ID NO: 12, and CDR3 comprises the amino acid sequence of SEQ ID NO: 13;
   vi. CDR1 comprises the amino acid sequence of SEQ ID NO: 1, CDR2 comprises the amino acid sequence of SEQ ID NO: 17, and CDR3 comprises the amino acid sequence of SEQ ID NO: 7;
   vii. CDR1 comprises the amino acid sequence of SEQ ID NO: 1, CDR2 comprises the amino acid sequence of SEQ ID NO: 6, and CDR3 comprises the amino acid sequence of SEQ ID NO: 7;
   viii. CDR1 comprises the amino acid sequence of SEQ ID NO: 1, CDR2 comprises the amino acid sequence of SEQ ID NO: 20, and CDR3 comprises the amino acid sequence of SEQ ID NO: 21;
   ix. CDR1 comprises the amino acid sequence of SEQ ID NO: 23, CDR2 comprises the amino acid sequence of SEQ ID NO: 6, and CDR3 comprises the amino acid sequence of SEQ ID NO: 24;
   x. CDR1 comprises the amino acid sequence of SEQ ID NO: 9, CDR2 comprises the amino acid sequence of SEQ ID NO: 26, and CDR3 comprises the amino acid sequence of SEQ ID NO: 27;
   xi. CDR1 comprises the amino acid sequence of SEQ ID NO: 9, CDR2 comprises the amino acid sequence of SEQ ID NO: 6, and CDR3 comprises the amino acid sequence of SEQ ID NO: 29;
   xii. CDR1 comprises the amino acid sequence of SEQ ID NO: 31, CDR2 comprises the amino acid sequence of SEQ ID NO: 32, and CDR3 comprises the amino acid sequence of SEQ ID NO: 33;
   xiii. CDR1 comprises the amino acid sequence of SEQ ID NO: 35, CDR2 comprises the amino acid sequence of SEQ ID NO: 36, and CDR3 comprises the amino acid sequence of SEQ ID NO: 37;
   xiv. CDR1 comprises the amino acid sequence of SEQ ID NO: 39, CDR2 comprises the amino acid sequence of SEQ ID NO: 40, and CDR3 comprises the amino acid sequence of SEQ ID NO: 37;
   xv. CDR1 comprises the amino acid sequence of SEQ ID NO: 35, CDR2 comprises the amino acid sequence of SEQ ID NO: 42, and CDR3 comprises the amino acid sequence of SEQ ID NO: 37;
   xvi. CDR1 comprises the amino acid sequence of SEQ ID NO: 39, CDR2 comprises the amino acid sequence of SEQ ID NO: 42, and CDR3 comprises the amino acid sequence of SEQ ID NO: 37;
   xvii. CDR1 comprises the amino acid sequence of SEQ ID NO: 39, CDR2 comprises the amino acid sequence of SEQ ID NO: 40, and CDR3 comprises the amino acid sequence of SEQ ID NO: 45;
   xviii. CDR1 comprises the amino acid sequence of SEQ ID NO: 47, CDR2 comprises the amino acid sequence of SEQ ID NO: 42, and CDR3 comprises the amino acid sequence of SEQ ID NO: 45;
   xix. CDR1 comprises the amino acid sequence of SEQ ID NO: 49, CDR2 comprises the amino acid sequence of SEQ ID NO: 42, and CDR3 comprises the amino acid sequence of SEQ ID NO: 45;
   xx. CDR1 comprises the amino acid sequence of SEQ ID NO: 51, CDR2 comprises the amino acid sequence of SEQ ID NO: 42, and CDR3 comprises the amino acid sequence of SEQ ID NO: 45;
   xxi. CDR1 comprises the amino acid sequence of SEQ ID NO: 53, CDR2 comprises the amino acid sequence of SEQ ID NO: 54, and CDR3 comprises the amino acid sequence of SEQ ID NO: 55;
   xxii. CDR1 comprises the amino acid sequence of SEQ ID NO: 57, CDR2 comprises the amino acid sequence of SEQ ID NO: 54, and CDR3 comprises the amino acid sequence of SEQ ID NO: 55;
   xxiii. CDR1 comprises the amino acid sequence of SEQ ID NO: 9, CDR2 comprises the amino acid sequence of SEQ ID NO: 54, and CDR3 comprises the amino acid sequence of SEQ ID NO: 55;
   xxiv. CDR1 comprises the amino acid sequence of SEQ ID NO: 60, CDR2 comprises the amino acid sequence of SEQ ID NO: 42, and CDR3 comprises the amino acid sequence of SEQ ID NO: 55;
   xxv. CDR1 comprises the amino acid sequence of SEQ ID NO: 62, CDR2 comprises the amino acid sequence of SEQ ID NO: 42, and CDR3 comprises the amino acid sequence of SEQ ID NO: 55;
   xxvi. CDR1 comprises the amino acid sequence of SEQ ID NO: 64, CDR2 comprises the amino acid sequence of SEQ ID NO: 42, and CDR3 comprises the amino acid sequence of SEQ ID NO: 55;
   xxvii. CDR1 comprises the amino acid sequence of SEQ ID NO: 47, CDR2 comprises the amino acid sequence of SEQ ID NO: 42, and CDR3 comprises the amino acid sequence of SEQ ID NO: 55;
   xxviii. CDR1 comprises the amino acid sequence of SEQ ID NO: 35, CDR2 comprises the amino acid sequence of SEQ ID NO: 42, and CDR3 comprises the amino acid sequence of SEQ ID NO: 55;
   xxix. CDR1 comprises the amino acid sequence of SEQ ID NO: 68, CDR2 comprises the amino acid sequence of SEQ ID NO: 42, and CDR3 comprises the amino acid sequence of SEQ ID NO: 55;
   xxx. CDR1 comprises the amino acid sequence of SEQ ID NO: 49, CDR2 comprises the amino acid sequence of SEQ ID NO: 42, and CDR3 comprises the amino acid sequence of SEQ ID NO: 55;
   xxxi. CDR1 comprises the amino acid sequence of SEQ ID NO: 1, CDR2 comprises the amino acid sequence of SEQ ID NO: 20, and CDR3 comprises the amino acid sequence of SEQ ID NO: 71;
   xxxii. CDR1 comprises the amino acid sequence of SEQ ID NO: 73, CDR2 comprises the amino acid sequence of SEQ ID NO: 74, and CDR3 comprises the amino acid sequence of SEQ ID NO: 75;
   xxxiii. CDR1 comprises the amino acid sequence of SEQ ID NO: 77, CDR2 comprises the amino acid sequence of SEQ ID NO: 78, and CDR3 comprises the amino acid sequence of SEQ ID NO: 79;
   xxxiv. CDR1 comprises the amino acid sequence of SEQ ID NO: 81, CDR2 comprises the amino acid sequence of SEQ ID NO: 82, and CDR3 comprises the amino acid sequence of SEQ ID NO: 83;
   xxxv. CDR1 comprises the amino acid sequence of SEQ ID NO: 31, CDR2 comprises the amino acid sequence of SEQ ID NO: 85, and CDR3 comprises the amino acid sequence of SEQ ID NO: 86;
   xxxvi. CDR1 comprises the amino acid sequence of SEQ ID NO: 88, CDR2 comprises the amino acid sequence of SEQ ID NO: 78, and CDR3 comprises the amino acid sequence of SEQ ID NO: 89;
   xxxvii. CDR1 comprises the amino acid sequence of SEQ ID NO: 91, CDR2 comprises the amino acid sequence of SEQ ID NO: 92, and CDR3 comprises the amino acid sequence of SEQ ID NO: 93;
   xxxviii. CDR1 comprises the amino acid sequence of SEQ ID NO: 95, CDR2 comprises the amino acid sequence of SEQ ID NO: 96, and CDR3 comprises the amino acid sequence of SEQ ID NO: 97;
   xxxix. CDR1 comprises the amino acid sequence of SEQ ID NO: 99, CDR2 comprises the amino acid sequence of SEQ ID NO: 100, and CDR3 comprises the amino acid sequence of SEQ ID NO: 101; or
   xl. CDR1 comprises the amino acid sequence of SEQ ID NO: 103, CDR2 comprises the amino acid sequence of SEQ ID NO: 104, and CDR3 comprises the amino acid sequence of SEQ ID NO: 105.
2. The antibody, or antigen-binding fragment thereof, of embodiment 1, wherein the VHH comprises an amino acid sequence having at least 90%, 91%, 92%, 93%, 94%, 95%, 96%, 97%, 98%, or 99% sequence identity to SEQ ID NO: 4, SEQ ID NO: 8, SEQ ID NO: 11, SEQ ID NO: 14, SEQ ID NO: 16, SEQ ID NO: 18, SEQ ID NO: 19, SEQ ID NO: 22, SEQ ID NO: 25, SEQ ID NO: 28, SEQ ID NO: 30, SEQ ID NO: 34, SEQ ID NO: 38, SEQ ID NO: 41, SEQ ID NO: 43, SEQ ID NO: 44, SEQ ID NO: 46, SEQ ID NO: 48, SEQ ID NO: 50, SEQ ID NO: 52, SEQ ID NO: 56, SEQ ID NO: 58, SEQ ID NO: 59, SEQ ID NO: 61, SEQ ID NO: 63, SEQ ID NO: 65, SEQ ID NO: 66, SEQ ID NO: 67, SEQ ID NO: 69, SEQ ID NO: 70, SEQ ID NO: 72, SEQ ID NO: 76, SEQ ID NO: 80, SEQ ID NO: 84, SEQ ID NO: 87, SEQ ID NO: 90, SEQ ID NO: 94, SEQ ID NO: 98, SEQ ID NO: 102, or SEQ ID NO: 106, provided that the VHH comprises the sequences of at least one set of CDR1, CDR2, and CDR3 sequences of embodiment 1.
3. The antibody, or antigen-binding fragment thereof, of embodiment 1, wherein the VHH comprises an amino acid sequence identical to SEQ ID NO: 4, SEQ ID NO: 8, SEQ ID NO: 11, SEQ ID NO: 14, SEQ ID NO: 16, SEQ ID NO: 18, SEQ ID NO: 19, SEQ ID NO: 22, SEQ ID NO: 25, SEQ ID NO: 28, SEQ ID NO: 30, SEQ ID NO: 34, SEQ ID NO: 38, SEQ ID NO: 41, SEQ ID NO: 43, SEQ ID NO: 44, SEQ ID NO: 46, SEQ ID NO: 48, SEQ ID NO: 50, SEQ ID NO: 52, SEQ ID NO: 56, SEQ ID NO: 58, SEQ ID NO: 59, SEQ ID NO: 61, SEQ ID NO: 63, SEQ ID NO: 65, SEQ ID NO: 66, SEQ ID NO: 67, SEQ ID NO: 69, SEQ ID NO: 70, SEQ ID NO: 72, SEQ ID NO: 76, SEQ ID NO: 80, SEQ ID NO: 84, SEQ ID NO: 87, SEQ ID NO: 90, SEQ ID NO: 94, SEQ ID NO: 98, SEQ ID NO: 102, or SEQ ID NO: 106.
4. The antibody, or antigen-binding fragment thereof, of any one of embodiments 1 to 3, wherein the VHH comprises an amino acid sequence having at least 90%, 91%, 92%, 93%, 94%, 95%, 96%, 97%, 98%, or 99% sequence identity, or is identical to, SEQ ID NO: 22, provided that CDR1 comprises the amino acid sequence of SEQ ID NO: 1, CDR2 comprises the amino acid sequence of SEQ ID NO: 20, and CDR3 comprises the amino acid sequence of SEQ ID NO: 21.
5. The antibody, or antigen-binding fragment thereof, of any one of embodiments 1 to 3, wherein the VHH comprises an amino acid sequence having at least 90%, 91%, 92%, 93%, 94%, 95%, 96%, 97%, 98%, or 99% sequence identity, or is identical to, SEQ ID NO: 43, provided that CDR1 comprises the amino acid sequence of SEQ ID NO: 35, CDR2 comprises the amino acid sequence of SEQ ID NO: 42, and CDR3 comprises the amino acid sequence of SEQ ID NO: 37.
6. The antibody, or antigen-binding fragment thereof, of any one of embodiments 1 to 3, wherein the VHH comprises an amino acid sequence having at least 90%, 91%, 92%, 93%, 94%, 95%, 96%, 97%, 98%, or 99% sequence identity, or is identical to, SEQ ID NO: 72, provided that CDR1 comprises the amino acid sequence of SEQ ID NO: 1, CDR2 comprises the amino acid sequence of SEQ ID NO: 20, and CDR3 comprises the amino acid sequence of SEQ ID NO: 71.
7. The antibody, or antigen-binding fragment thereof, of any one of embodiments 1 to 6, wherein the antibody, or antigen-binding fragment thereof is a single domain antibody (sdAb), a nanobody, or an isolated VHH domain.
8. The antibody, or antigen-binding fragment thereof, of any one of embodiments 1 to 7, wherein the antibody or antigen-binding fragment thereof binds to human survivin.
9. The antibody, or antigen-binding fragment thereof, of any one of embodiments 1 to 8, wherein the antibody or antigen-binding fragment thereof binds to human survivin on the surface of B-cells.
10. An isolated nucleic acid molecule encoding an antibody, or antigen-binding fragment thereof, of any one of embodiments 1 to 9.
11. An expression vector comprising the nucleic acid molecule of embodiment 10.
12. A host cell comprising the nucleic acid molecule of embodiment 10 or the expression vector of embodiment 11.
13. A pharmaceutical composition comprising an antibody, or antigen-binding fragment thereof, of any one of embodiments 1 to 9.
14. The pharmaceutical composition of embodiment 13, wherein the pharmaceutical composition is formulated for intravenous or subcutaneous injection.
15. The pharmaceutical composition of embodiment 13, wherein the pharmaceutical composition is an injectable pharmaceutical composition.
16. A kit comprising:
   a) an antibody, or antibody binding fragment of any one of embodiments 1 to 9,
   b) an isolated nucleic acid molecule of embodiment 10,
   c) an expression vector of embodiment 11, and/or
   d) a pharmaceutical composition of embodiments 12 to 15, and packaging for the same.
17. A method of producing an antibody, or antigen-binding fragment thereof, that binds human survivin, the method comprising:
   (a) growing or culturing the host cell of embodiment 12 under conditions so that the host cell expresses a polypeptide or polypeptides comprising a heavy chain variable domain (VHH), thereby producing the antibody or the antigen-binding fragment of the antibody; and
   (b) purifying the antibody, or the antigen-binding fragment thereof.
18. A method of treating a survivin-mediated disorder in a subject in need thereof, the method comprising administering to the subject an antibody, or antigen-binding fragment thereof, of any one of embodiments 1 to 9 or a pharmaceutical composition of any one of embodiments 13 to 15.
19. The method of embodiment 18, wherein the survivin-mediated disorder is an autoimmune disorder.
20. The method of embodiment 19, wherein the autoimmune disorder is or comprises antineutrophil cytoplasmic antibody (ANCA)-associated vasculitis, alopecia areata, ankylosing spondylitis, antiphospholipid syndrome, Addison's disease, hemolytic anemia, autoimmune hepatitis, hepatitis, Behcets disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, systemic lupus erythematosus, Meniere's Disease, mixed connective tissue disease, multiple sclerosis, myasthenia gravis, neuromyelitis optica, pemphigus vulgaris, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjogren's syndrome, stiff-man syndrome, systemic sclerosis, Takayasu arteritis, temporal arteritis, ulcerative colitis, uveitis, vitiligo, and Wegener's granulomatosis.
21. The method of embodiment 19 or 20, wherein the autoimmune disorder is myasthenia gravis.
22. The method of embodiment 18, wherein the survivin-mediated disorder is a cancer.
23. The method of embodiment 22, wherein the cancer is or comprises breast cancer, kidney cancer, liver cancer, lung cancer, malignant glioma, melanoma, multiple myeloma, neuroendocrine tumors (NETs), pediatric brain tumors (gliomas), and prostate cancer.

### EXAMPLES

The following examples are provided to further describe some of the aspects and embodiments disclosed herein. The examples are intended to illustrate, not to limit, the disclosed aspects or embodiments.

### Example 1: Preliminary Development of Targeted Immunotherapy Against Survivin

### A. Survivin and its Role in Autoimmune Diseases

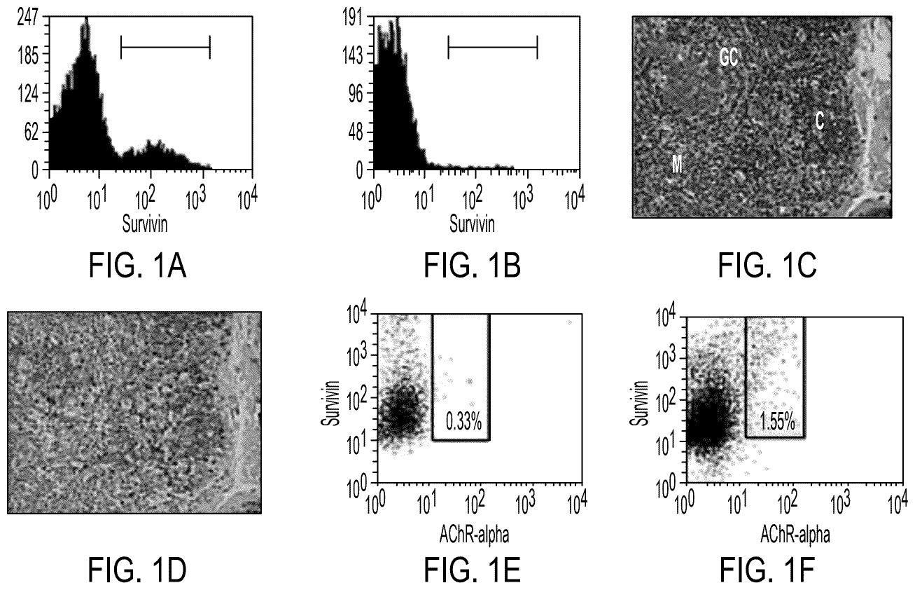

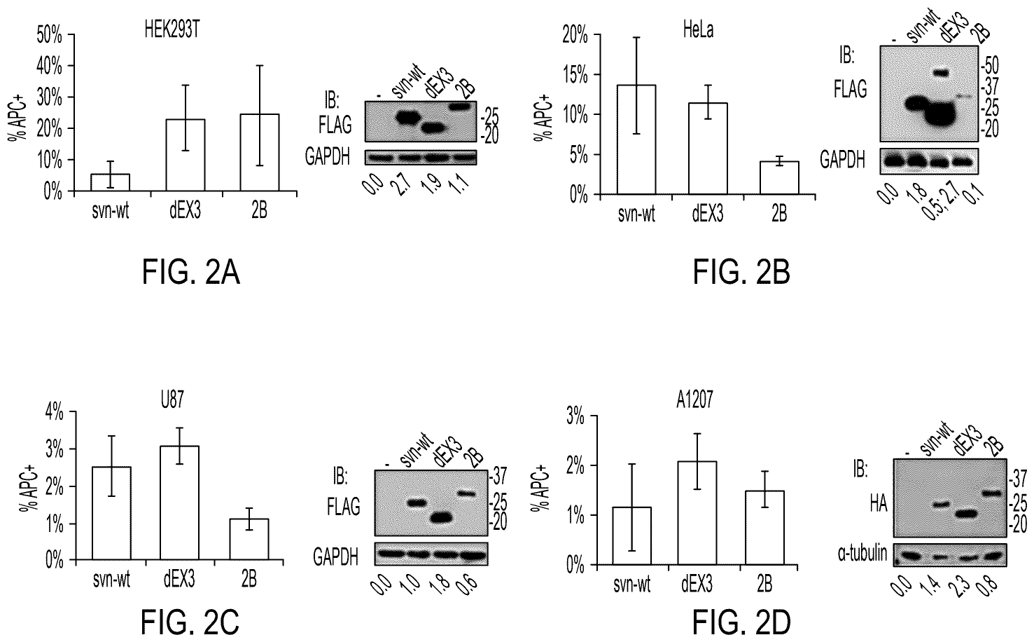

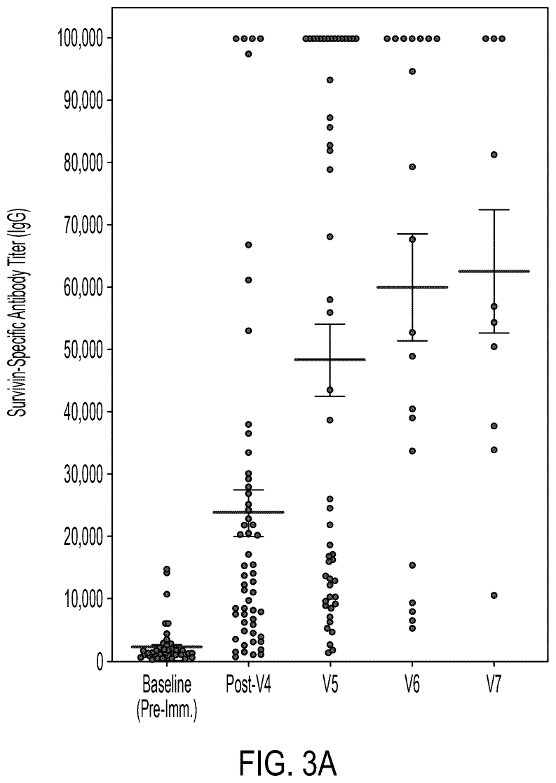

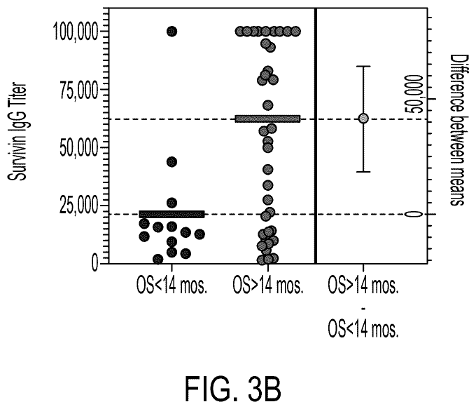

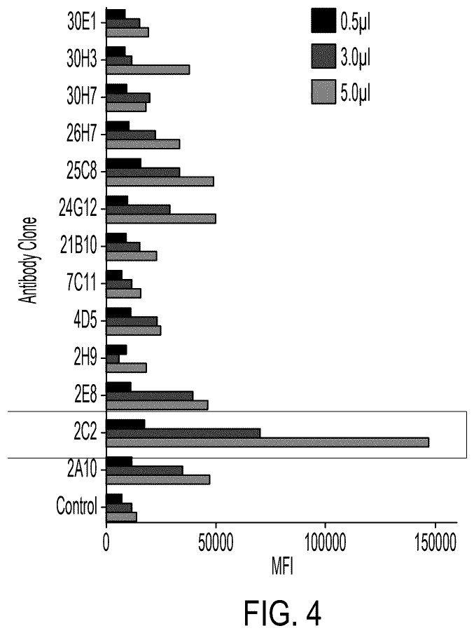

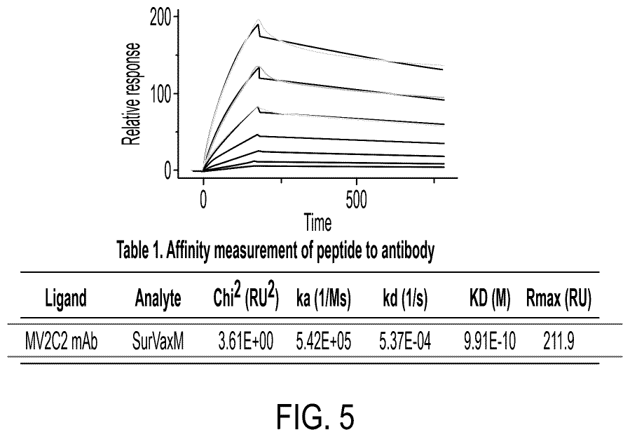

Survivin, an anti-apoptotic protein, initially identified as critical in cancer cell survival, has been implicated in autoimmune diseases such as MG, rheumatoid arthritis, systemic sclerosis, systemic lupus erythematosus, and ulcerative colitis. Investigators have previously demonstrated the overexpression of survivin in the B-lymphocyte lineage plasma cells and in the thymus of MG patients relative to controls. FIG. 1A-1B depict fluorescence-activated cell sorting (FACS) analysis of total survivin expression in peripheral blood mononuclear cells (PBMCs) from a myasthenic patient (FIG. 1A) and a healthy control (FIG. 1B). FIGS. 1C-1D depict representative images of hematoxylin and eosin (H&E) staining (FIG. 1C) and survivin-positive staining (FIG. 1D) in thymus samples from a 24-year-old female patient with early-onset MG. Analysis of the thymus from the MG patient shows a higher proportion of survivin-positive cells when compared to the thymus from corticosteroid-treated MG patients. Furthermore, it has been shown that the a subunit of the acetylcholine receptor (AChR) peptide specifically binds to survivin-positive B-cells derived from MG patients, supporting the hypothesis that a survivin-positive B-cell subset is implicated in the production of autoantibodies. FIG. 1E-1F depict the binding of the a subunit of acetylcholine receptor (AChR) peptide to PBMCs from controls (FIG. 1E) and MG patients (FIG. 1F) as determined by five-color FACS analysis.

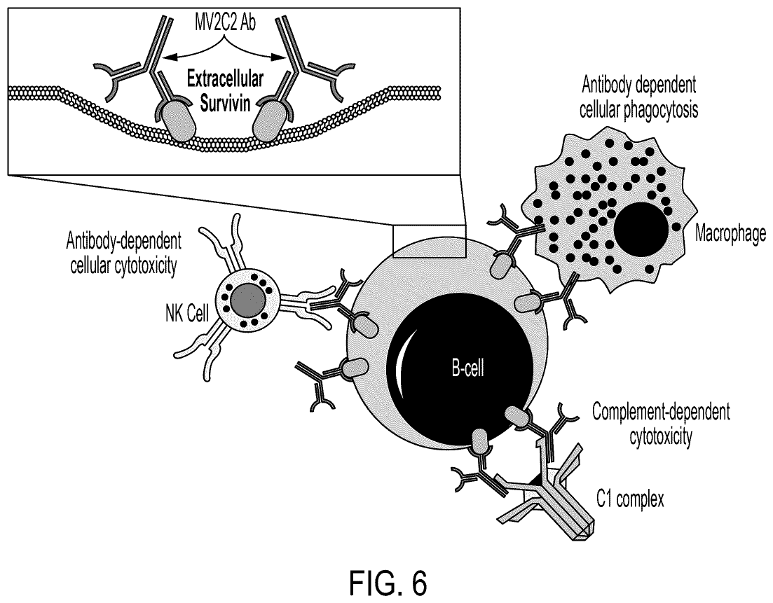

Survivin has been traditionally regarded as an intracellularly expressed protein. However, recent investigations have revealed the extracellular localization of survivin by detecting isoforms that interact with extracellularly positioned tubulin on cancer cells. Analysis of survivin-positive B-cells indicates that CD20+ cells from MG patients exhibit a higher percentage of extracellular survivin that those from healthy controls. Examination of CD4+ cells show an increased percentage of intracellular survivin in MG patients as compared to healthy controls, while the percentage of extracellular survivin among CD4+ cells was unaffected. Additional studies have demonstrated the extracellular localization of survivin by detecting survivin isoforms that interact with extracellular positioned tubulin on cancer cells. Specifically, FIGS. 2A-2D depict flow cytometry detection of survivin isoforms (N-terminal FLAG-HA constructs) in non-permeabilized cells. Collectively, these results indicate survivin as a new specific therapeutic target for MG.

Based on this preliminary data, targeting survivin is anticipated to be specific to pathogenic B-cells, which are identified by the expression of surface survivin. The response mechanism is predicated on the binding of the AChR peptide to survivin-positive cells in both animal models and humans. Differing from current CD20 ablation therapies, survivin-focused ablation is expected to target a smaller subset of lymphocytes. Treatment with anti-survivin antibodies is projected to be logistically straightforward and applicable to all patients.

### B. Development of Survivin-Targeting Antibody

Leveraging the extracellular expression of survivin in the B-cell subpopulation reactive to MG as a targetable marker, a prototype antibody that selectively recognizes and targets survivin-positive cells was developed: MV2C2.

The MV2C2 antibody originated from the SVN53-67/M57 (SurVaxM) immunizing antigen, which is currently undergoing clinical trials for glioblastoma. MV2C2 embodies the isolated humoral component of the SurVaxM-induced immune response, obviating the need to immunize patients directly. While vaccines provide one way forward in anti-survivin cancer immunotherapy, there are several advantages of using a passive immunotherapy with antibodies. For example, a humanized monoclonal antibody: 1) would not be HLA-restricted (unlike peptide vaccines), 2) could potentially have immediate action against cancer cells in patients who are severely immunocompromised by their tumors, 3) would be doseable, and 4) could be used in conjunction with a vaccine or other drugs or therapies such as, for example, radiation therapy, to exploit alternative or complementary mechanisms of action. One or more the above advantages are also applicable to mAbs in general, including chimeric, human and humanized antibodies.

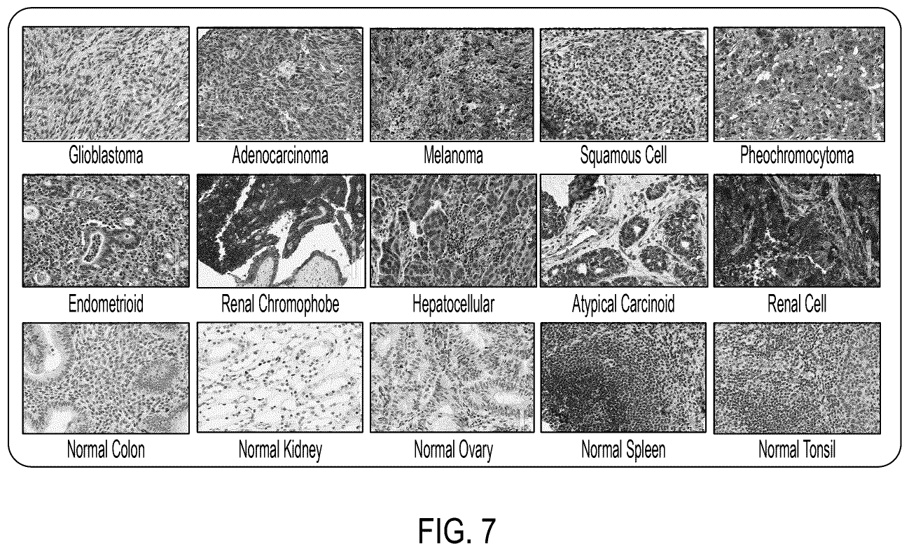

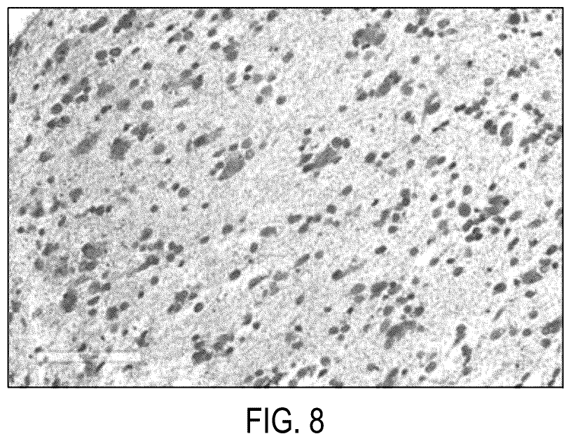

Anti-SurVaxM monoclonal antibodies (mAbs) were developed after observing patient antibodies in SurVaxM clinical trials. FIG. 3A depicts the titers of anti-survivin specific antibodies (IgG) across different time points throughout the SurVaxM vaccination schedule. At baseline (pre-immunization), the titers are uniformly low, whereas a notable escalation is evident post-fourth vaccination (Post-V4), and this upward trend persists at subsequent time points V5, V6, and V7. The data suggests a variable but generally positive immunological response to the vaccination regimen. FIG. 3B contrasts the anti-survivin IgG titers between two cohorts categorized by overall survival (OS), one with OS≤months and the other with OS>14 months. The latter group exhibited significantly higher antibody titers. This correlation between increased survivin-specific IgG titers and extended overall survival was hypothesized to indicate a beneficial prognostic implication of anti-SurVaxM mAbs.

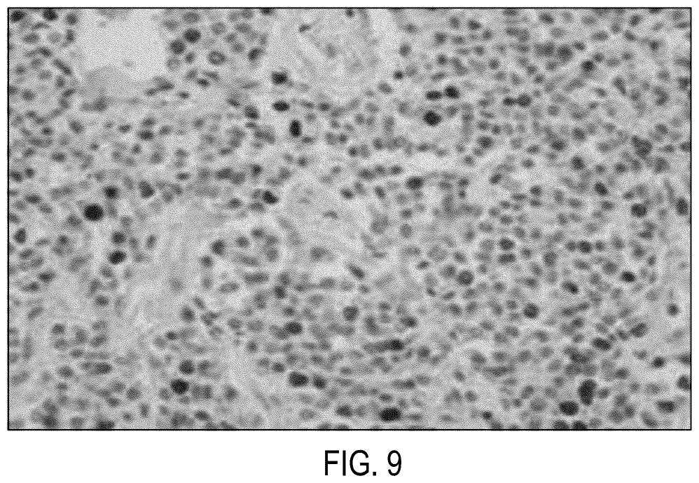

Anti-SurVaxM mAbs were generated through the immunization of mice with a modified survivin peptide. This development process resulted in a variety of anti-SurVaxM mAb clones. Among these, the clone designated MV2C2 demonstrated the highest affinity for survivin and was subsequently selected for further development. As depicted in FIG. 4, the mean fluorescence intensity (MFI) values for MV2C2, represented in three different volumes exceeded those of other clones, indicating a superior binding capability. The graph delineates the performance of each clone at varying concentrations, with MV2C2's binding affinity distinctly higher even at the lowest volume, as compared to the control and other antibody clones.

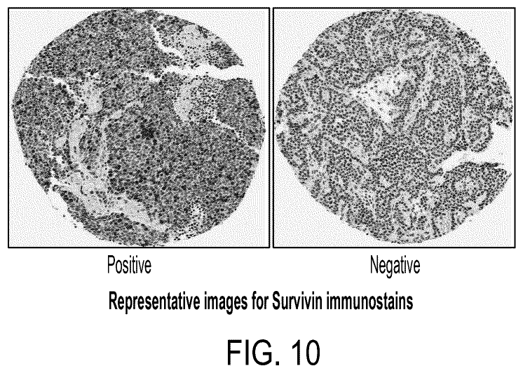

The MV2C2 antibody is specifically engineered to target a conserved region within exon 2 of the wild type survivin sequence, a region common to all seven described survivin isoforms. This design ensures that the antibody concurrently targets the complete “family” of survivin isoforms. Specifically, the MV2C2 monoclonal IgG binds a conserved 15 amino acid survivin epitope (SVN53-67) present in the several known human survivin isoforms (as well as having 100% homology to the same region in mouse survivin). FIG. 5 shows that the MV2C2 antibody binds to survivin with a high affinity, evidenced by a dissociation constant (KD) of 9.9×10⁻¹⁰ M, a value within the optimal range for therapeutic antibodies. Such a level of affinity suggests the potential for the MV2C2 antibody to activate multiple immune pathways against survivin-positive B-cells. These pathways include antibody-dependent cellular phagocytosis by macrophages, complement activation, and antibody-dependent cellular cytotoxicity, which involves the activation of natural killer (NK) cells. FIG. 5 depicts a schematic representation of the possible mechanisms of action of MV2C2.

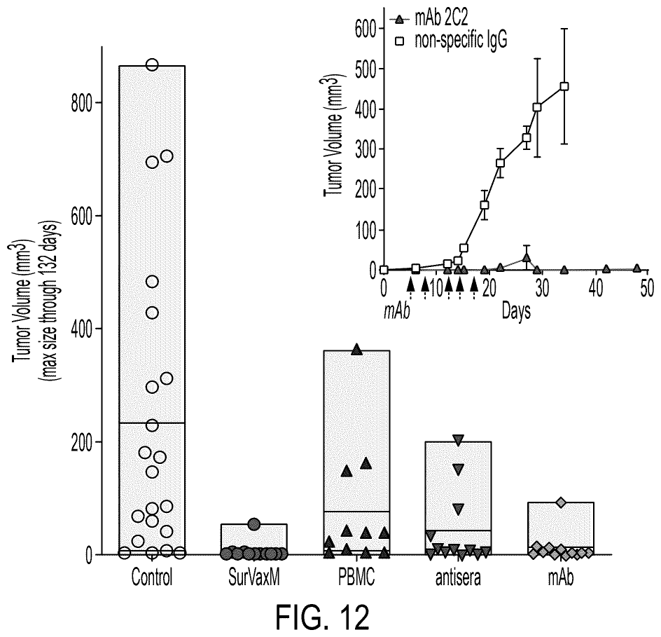

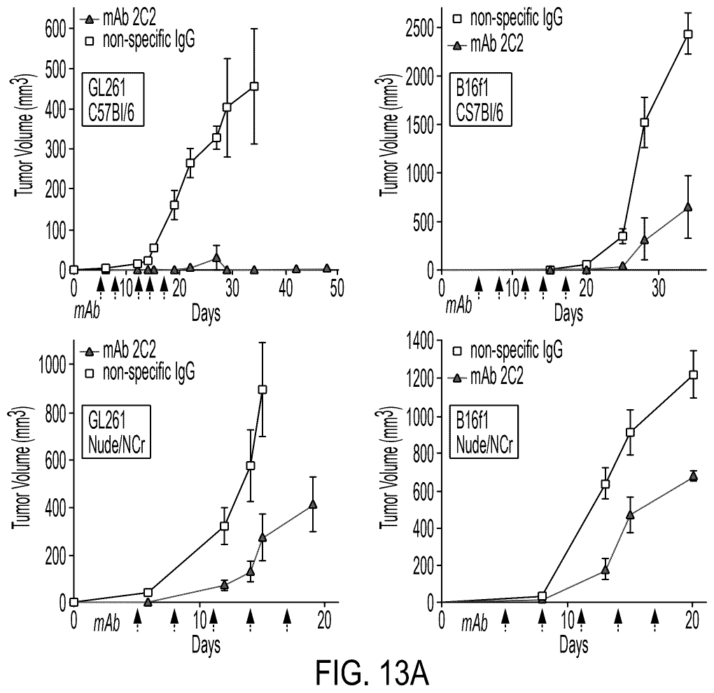

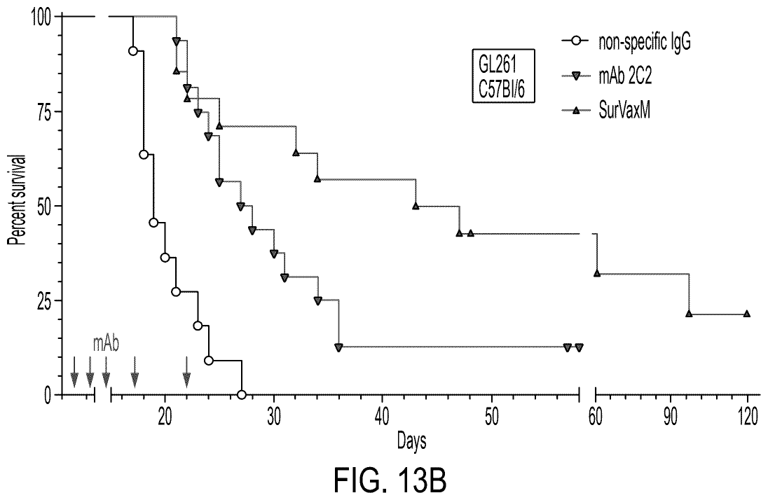

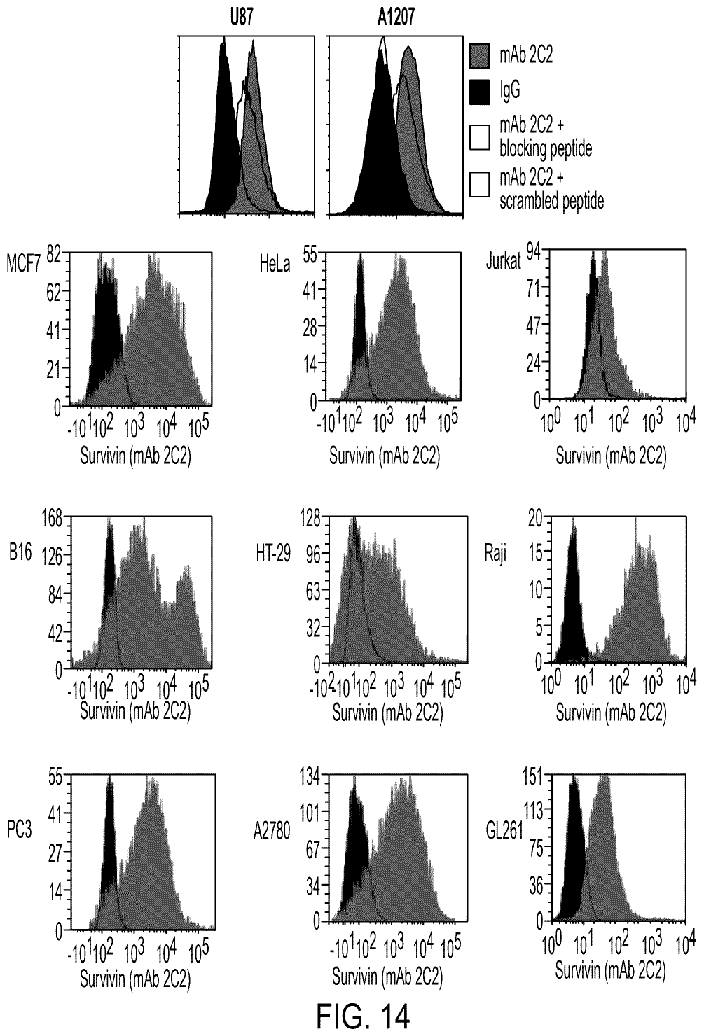

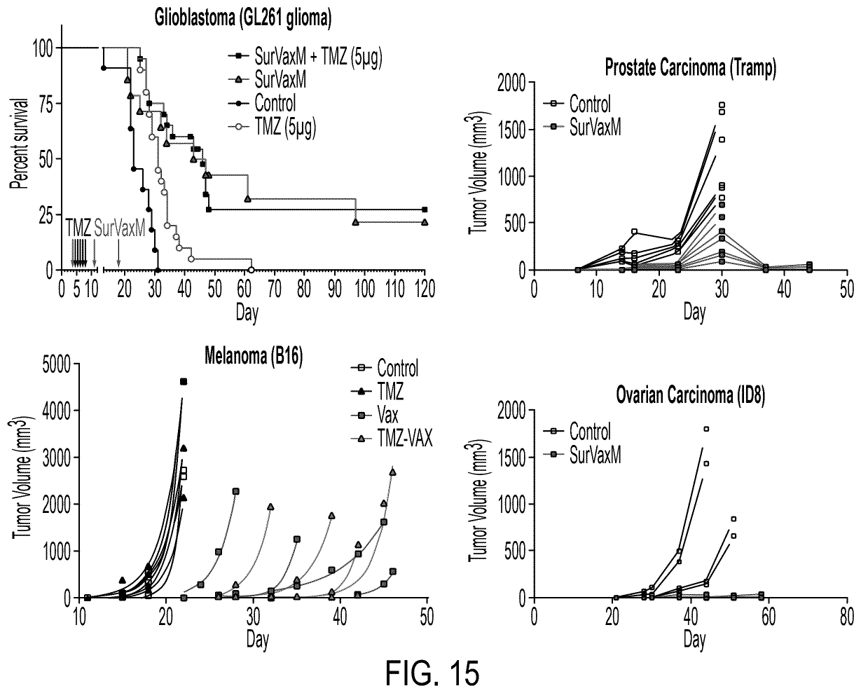

Histological analysis using the MV2C2 antibody has further demonstrated its broad reactivity across various tumor types. As depicted in FIG. 7, MV2C2 prominently stains a variety of cancers, including glioblastoma, adenocarcinoma, and melanoma, suggesting a widespread over-expression of survivin in these malignancies. This pattern corroborates survivin's role as a pan-cancer target, with its expression typically associated with aggressive and rapidly proliferating tumor cells. FIGS. 8-9 depict the use of MV2C2 in detecting survivin expression in lower-grade glioma and multiple myeloma tissues, respectively. FIG. 10 depicts the effectiveness of MV2C2 in neuroendocrine tumor (NET) tissue microarray analysis (TMA), where a significant fraction of the tissues displayed survivin positivity. Moreover, the differential expression pattern depicted in FIG. 11, which contrasts survivin expression in various tissues including breast, liver, and thyroid cancers, emphasizes the potential of MV2C2 in distinguishing malignant from non-malignant tissue. Collectively, these finding highlight survivin's presence in cells across a range of tumor types, and MV2C2's potential therapeutic application targeting survivin-expressing malignant cells.

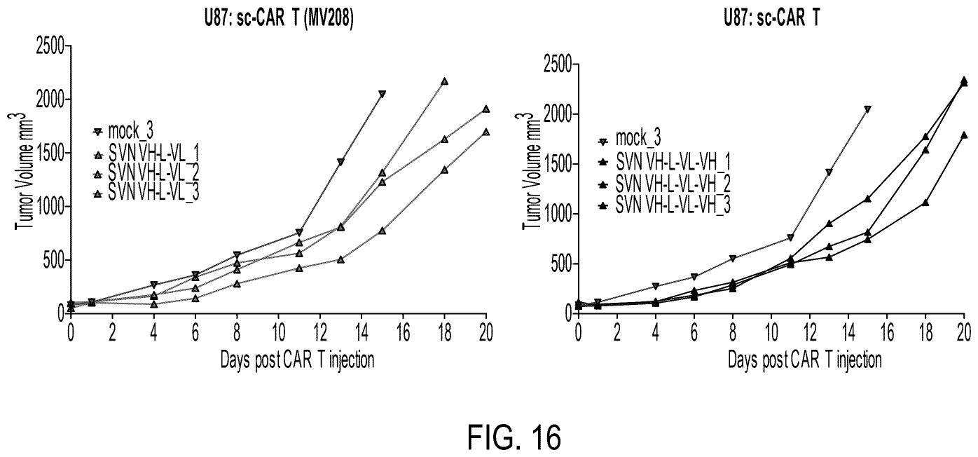

In further experiments murine experiments, the MV2C2 anti-survivin antibody utilized SurVaxM as the antigen, recreating patient-observed immune responses for further examination. A GL261 glioma cell model was grown in C57BL/6 mice. The results are depicted in FIG. 12. PBMCs, which were adoptively transferred from SurVaxM-immunized mice to naïve mice, demonstrated a retention of anti-tumor activity. Similarly, the transfer of SurVaxM antisera into naïve mice preserved some level of anti-tumor activity. Notably, the purified mAb IgG exhibited potent anti-tumor activity in the absence of any T cells, suggesting a direct effect of the antibody on the tumor cells. This effect is facilitated by the presence of survivin isoforms at the cell surface, which are accessible to the antibody-mediated immune mechanisms. The bar graph in FIG. 12 delineates the maximum tumor volume through 32 days across various treatment groups, with the purified mAb IgG group showing a significant reduction in tumor growth compared to controls, further indicating the potential efficacy of the antibody treatment.

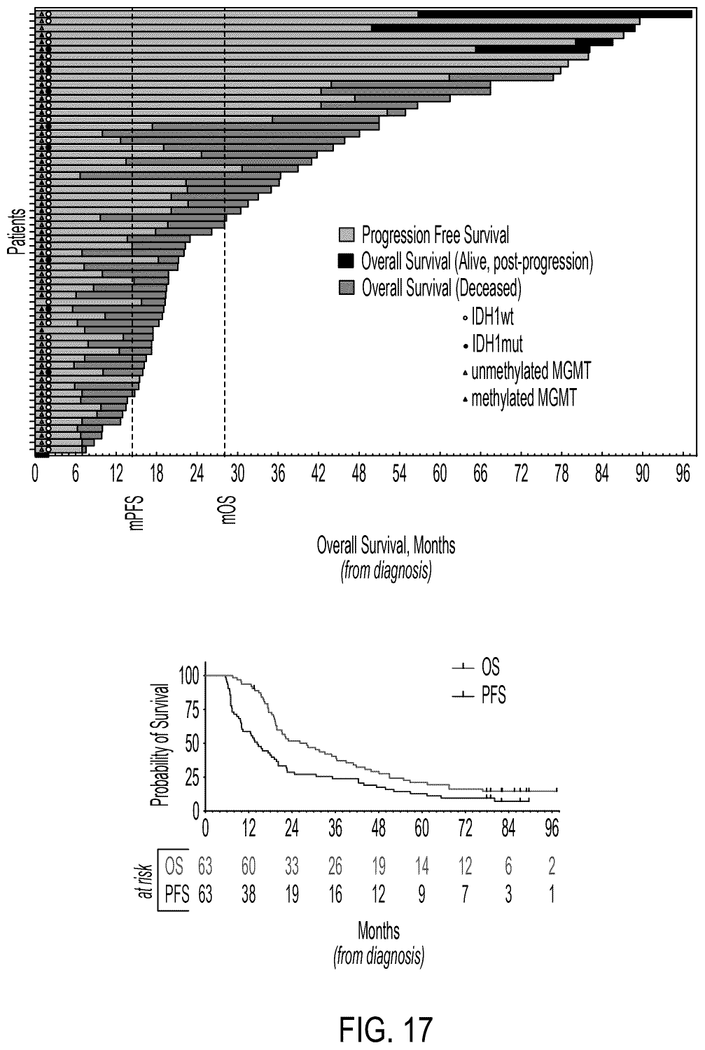

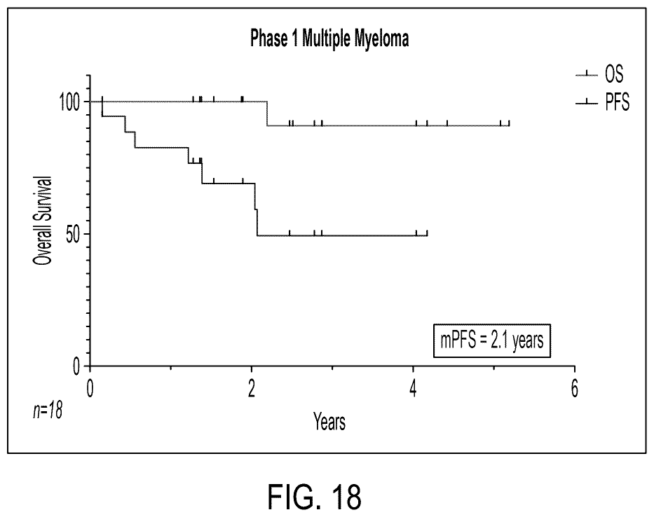

FIGS. 13A-13B depict the results from additional preclinical studies, assessing anti-SurVaxM derived mAb IgG for their capability to control tumor growth in subcutaneous (SQ) models of glioma and melanoma. The cloned mAbs were employed against both GL261 glioma and B16F1 melanoma cell lines. Specifically, the GL261 glioma model, hosted in C57BL/6 mice and in an immunodeficient Nude/Ncr mouse strain, responded to the mAb IgG treatment, as observed in the reduced tumor volumes over a span of 20 to 30 days. FIG. 13A depicts a comparison of tumor growth inhibition between the mAb 2C2 and non-specific IgG across different mouse strains and tumor types, with mAb 2C2 consistently demonstrating a potent suppression of tumor expansion. Further corroborating these findings, FIG. 13B depicts the survival curves of the C57BL/6 mice inoculated with GL261 cells and treated with either non-specific IgG, mAb 2C2, or SurVaxM. The survival analysis indicates that mice treated with mAb 2C2 had a significantly prolonged survival rate compared to those treated with non-specific IgG. This is indicative of the therapeutic potential of mAb 2C2, not only in tumor size reduction but also in enhancing survival.

### C. Specificity and Efficacy of MV2C2 in Various Cancer Models

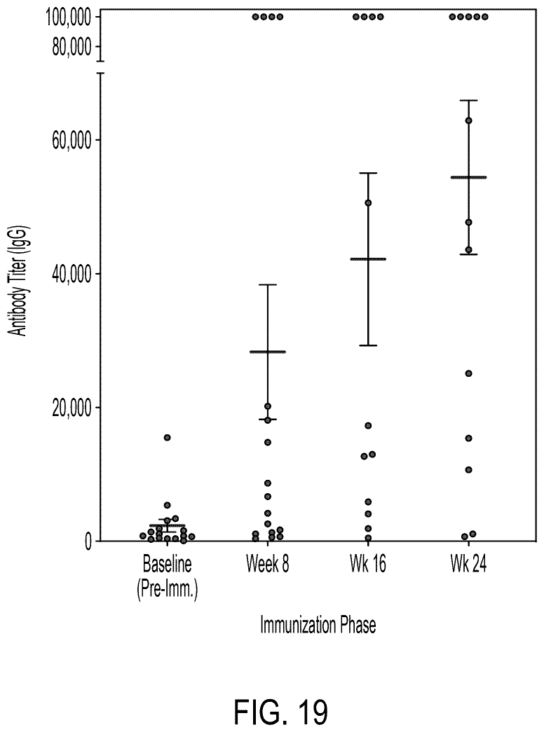

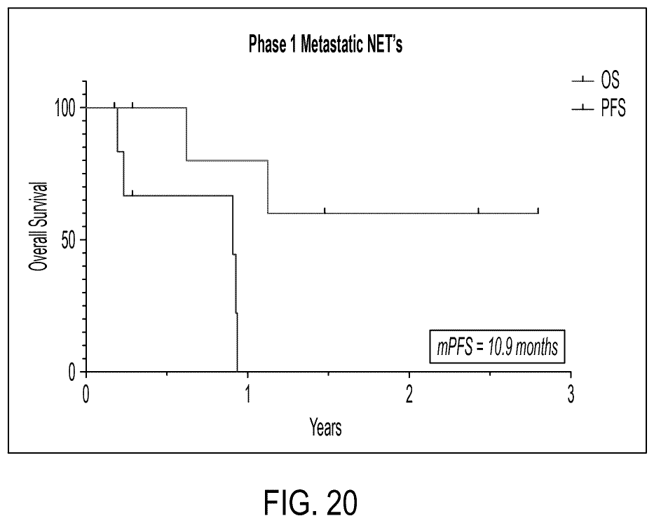

The specific and efficacy of the MV2C2 antibody against survivin-expressing cancer cells were evaluated through flow cytometry and tumor growth inhibition studies, as depicted in FIGS. 14-15. Flow cytometry analysis demonstrated that MV2C2 binds specifically to survivin-expressing cells across a variety of cancer cell lines, including U87 glioblastoma and A1207 melanoma cells. The specificity of the antibody was further confirmed by the inhibition of binding in the presence of a blocking peptide, whereas a scrambled peptide did not affect the binding, illustrating the targeted interaction of MV2C2 with the survivin epitope.

Further preclinical studies assessed the therapeutic efficacy of MV2C2 in inhibiting tumor growth in vivo. These studies utilized multiple animal models, including glioblastoma (GL261), melanoma (B16), prostate carcinoma (Tramp), and ovarian carcinoma (ID8). The data depicted in FIG. 15 indicates that treatment with MV2C2 inhibits tumor growth compared to controls, across all tested models. Notably, in the glioblastoma model, animals treated with MV2C2 exhibited extended survival times, while in melanoma models, the antibody treatment reduced tumor growth rates. The prostate and ovarian cancer models further demonstrated the capability of MV2C2 to control and reduce tumor progression, confirming its broad-spectrum efficacy against different types of survivin-positive malignancies.

### D. Characterization of MV2C2 in the EAMG Mouse Model

Investigators analyzed the MV2C2 antibody's binding specificity and affinity for survivin-positive B-cells. An EAMG model was generated to procure lymphocytes for the experiment. Six mice were injected with purified acetylcholine receptor (AChR) emulsified in complete Freund's adjuvant (CFA) at a concentration of 40 micrograms in 200 microliters, while the control group of six mice received saline/CFA. On days 28 and 56, booster injections of AChR in incomplete Freund's adjuvant (IFA) were administered to the EAMG animals; the control group received saline/IFA injections. All groups were sacrificed upon the observation of weakness in the EAMG group. Lymphocytes from the blood, spleen, and lymph nodes were then harvested for analysis.

The samples from both EAMG and control animals were characterized using flow cytometry to identify the different cell populations, including B-cells, plasma cells, and T-cells. The analysis determined the percentage composition and the presence of specific cluster of differentiation (CD) molecules using population-specific antibodies. Additionally, tetramer staining for AChR was used to pinpoint autoreactive cells. Comparative analysis of the MV2C2 antibody and two other clones against isotype controls was conducted to evaluate the inhibition of the immune response in AChR recall assays and ELISPOT for interferon-gamma on samples from both EAMG and control animals, thereby verifying the antibodies' response specificity. Lymphocyte cultures were incubated with AChR to provoke a recall response, whereupon proliferation, cell viability, and interferon-gamma production were assessed. Lastly, the impact of the MV2C2 antibody prototype and antibody clones on autoantibody reduction was evaluated in lymphocyte cultures from EAMG and control animals. An enzyme-linked immunosorbent assay (ELISA) measured the production of AChR autoantibodies in these cultures. The MV2C2 antibody and the most efficacious clone in inhibiting the AChR autoantibody response by over 20% were selected for further study.

Investigators next assessed the therapeutic potential of the MV2C2 antibody and a selected antibody clone. The objective was to verify the rescue of the MG phenotype in EAMG mice and to elucidate the therapeutic effects of the MV2C2 antibody relative to the antibody clone selected above, which recognizes survivin. EAMG mice of both sexes were included in a 1:1 male-to-female ratio to provide a representative cross-section of the general patient population.

Three groups of EAMG mice-those treated with MV2C2, an alternative clone, and an untreated group-along with a control group induced with saline, were compared, with eight mice per group. The dosing frequency was set at every other day for six doses. The reversal of the MG phenotype was evaluated using both a disease score and forelimb grip strength measurements. Additionally, spleens, lymph nodes, and blood were collected to conduct subsequent in-depth phenotypical flow cytometric and functional analyses to study the effects of the MV2C2 antibody.

The MV2C2 antibody and the antibody clone exhibited binding with greater than 95% affinity to survivin-positive cells, and the key sequences necessary for humanization were successfully identified. Disease scores registered at less than 1 for both the MV2C2 antibody and the antibody clone. The achieved milestones included the MV2C2 antibody prototype exhibiting higher affinity and specificity in recognizing survivin compared to other selected clones, the determination of the MV2C2 antibody isotype, and the confirmed rescue of the MG phenotype in EAMG mice.

To summarize, the administration of MV2C2 antibody to EAMG mice significantly lowered the number of circulating autoantibodies and restored AChR levels at the neuromuscular junction (NMJ) of treated mice. Further experimentation involving the administration of MV2C2 antibody to both mice and rats with EAMG resulted in improved motor function assessments, a reduction in AChR-specific autoantibodies, and the preservation of AChR at the NMJ. These effects were associated with a decrease in survivin-positive CD20-positive cells and a reduction in CD19-positive survivin-positive splenocytes, thereby reinforcing the antibody's therapeutic potential without evidencing toxicity.

### E. Assessment of MV2C2 Mechanism of Action in Human Blood Samples

Investigators first assessed MV2C2 antibody binding to the survivin-positive B-cell subset. To verify that the MV2C2 antibody specifically recognized survivin-positive B-cells and to further characterize this population, PBMCs were sorted and collected from 50 MG patients (30 AChR-positive MG and 15 muscle-specific tyrosine kinase (MuSK) positive MG) and 30 control subjects without autoimmunity. The affinity of the MV2C2 antibody was verified using a fluorescent cell-based assay, and its specificity was confirmed by blocking the binding site with an anti-survivin clone. The PBMCs were processed using a BD Influx high-speed cell sorter to assess extracellular survivin expression. To verify the specific affinity of the MV2C2 antibody for this subpopulation, Western blot (WB) and immunohistochemistry (IHC) analyses were performed, using samples from non-autoimmune subjects as a negative control. Half of the sample tested for WB and IHC was used to conduct single-cell and bulk-cell sequencing to determine the specific subset of cells expressing a high percentage of survivin and recognizable by the MV2C2 antibody. The binding of the antibody to the lymphocytes and its mode of action were determined. The binding of survivin-positive cells to peptides from AChR and MuSK was confirmed. FITC-labeled AChR al subunit peptide and the extracellular peptide of MuSK were evaluated for their ability to bind to PBMCs from MG patients. Peptide binding was detected by flow cytometry. To ensure specificity of binding, unlabeled AChR and MuSK peptides were deployed.

Investigators next focused on identifying the molecular mechanisms of antibody-mediated action on a specific B-cell population. To discern the specific pathways activated by the binding of the MV2C2 antibody, investigators performed three assays related to the pathways induced by antibody binding on survivin-positive cells: 1) an antibody-dependent cellular cytotoxicity (ADCC) assay; 2) a complement-dependent cytotoxicity (CDC) assay; and 3) a chromatin condensation assay. Commercially available kits were used to conduct these assays with five samples from AChR-positive MG patients and three samples from healthy controls.

Investigators then assessed the effect of the MV2C2 antibody on an experimental antigen using an antibody capture enzyme immunoassay. In this assay, MV2C2 antibody was used to coat microtiter plates, and chicken ovalbumin (OVA) expressing cytomegalovirus (CMV) infected or uninfected human natural killer (NK) cells were added to duplicate wells. The plates were then incubated and subsequently washed; cells were permeabilized, and a biotinylated antibody targeting OVA was added to each replicate, followed by colorimetric development. For each sample, the difference in mean optical density between the antigen-positive wells and the negative control wells was calculated. The immune effector pathways (ADCC, CDC, cytotoxicity) associated with MV2C2 antibody binding to survivin-positive B-cells were upregulated. The MV2C2 antibody demonstrated greater than 90% specificity for the extracellular survivin-positive B-cell population that produces autoantibodies, such as anti-AChR and anti-MuSK, characteristic of MG. Human samples were used to confirm that the survivin-positive population binds peptides from AChR and MuSK. The phenotype of the human survivin-positive B-cell subset recognized by the MV2C2 antibody was described, and its association with AChR and MuSK binding was confirmed.

In sum, the above experiments describe findings related to the MV2C2 antibody's mechanism of action in targeting survivin-positive B-cells in human blood samples from MG patients. MV2C2 showed efficacy in binding specific targets and enhancing immune effector pathways, and underscores targeting survivin in clinical applications for autoimmune diseases such as MG. This example provides a foundation for the development of sdAbs that can achieve high specificity with minimal off-target effects, as described below.

### F. Development of CAR-T Cell Therapy Using MV2C2

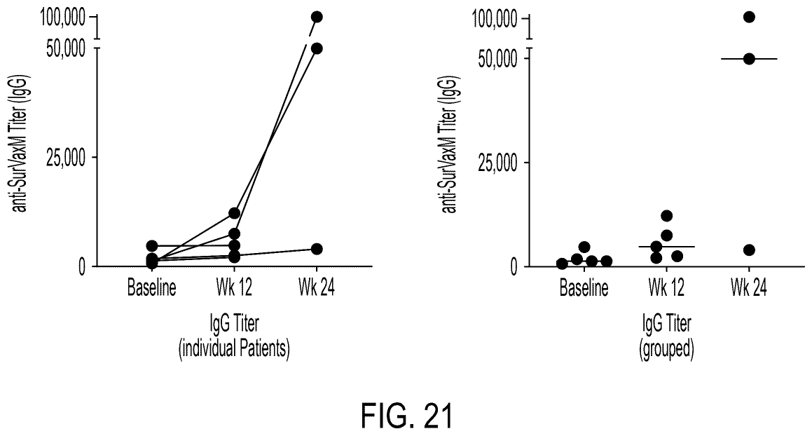

Extending the therapeutic utility of the MV2C2 antibody, investigators explored its application as a chimeric antigen receptor (CAR) in T-cell therapy, particularly targeting survivin-expressing glioma cells. Utilizing MV2C2 as the CAR receptor, preclinical models demonstrated significant efficacy in CAR-T cell therapy against U87 glioma models. As depicted in FIG. 16, two different constructs of CAR-T cells, employing the single-chain variable fragment (scFv) of MV2C2, were assessed. These constructs included the MV2C2 sc-CAR T and the MV2C2 sc-CAR T in combination with survivin-binding domains enhancing the targeting and cytotoxic capacity of the T cells.

CAR-T cells expressing the MV2C2 receptor showed a potent anti-tumor activity compared to mock treatments. FIG. 16 depicts tumor growth inhibition by MV2C2 sc-CAR T cells, demonstrating a marked reduction in tumor growth rates. FIG. 16 also compares the performance of different constructs of MV2C2-based CAR-T cells against the U87 glioma model, indicating that all constructs outperformed the control group, with variable efficacy among the constructs. These results not only validate MV2C2's high specificity and affinity towards survivin-expressing cells but also underscore its potential to be repurposed as a CAR-T cell receptor.

### G. Clinical Outcomes of SurVaxM in Oncology Trials

Clinical trials employing SurVaxM, the immunogenic progenitor of MV2C2, provide insights into the therapeutic impact of survivin targeting therapies across various cancer types, including glioblastoma (GBM), multiple myeloma (MM), and neuroendocrine tumors (NETs).

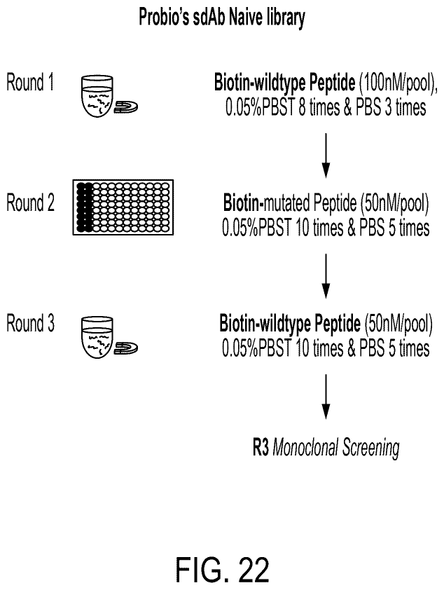

The Phase 2a study of SurVaxM in newly diagnosed GBM patients illustrated promising outcomes, with overall survival reaching 28.1 months from diagnosis, exceeding the expected median of 15-18 months. Progression-free survival was also prolonged, recorded at 14.4 months compared to the anticipated 4-7 months, as depicted in FIG. 17.

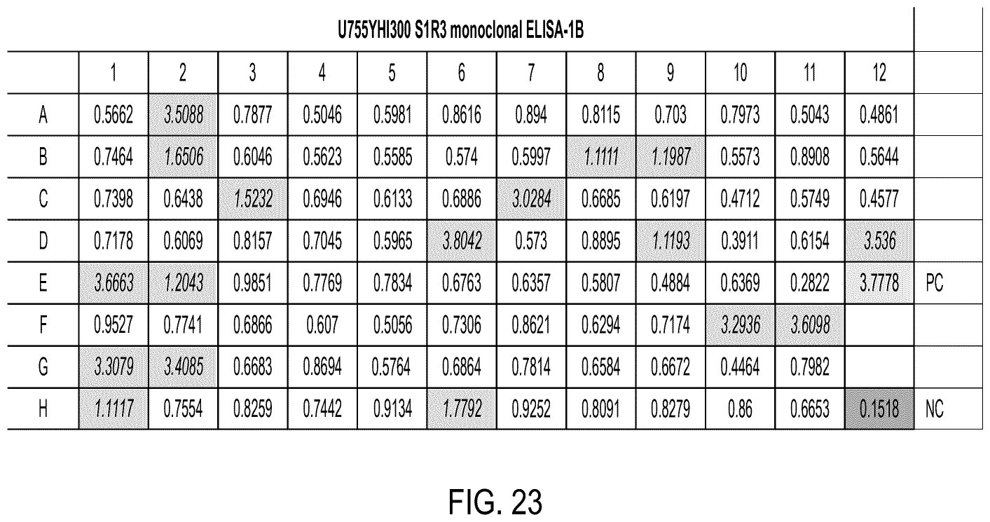

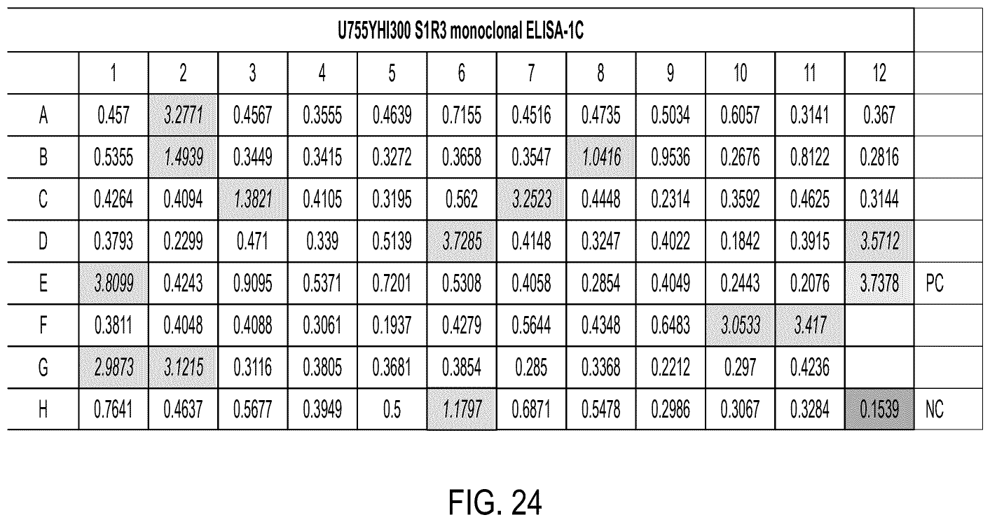

In a Phase I trial targeting MM, SurVaxM was administered either before or following lenalidomide maintenance therapy. The outcomes demonstrated that SurVaxM was well-tolerated and had a favorable immunological and therapeutic profile. At the data cut-off, the median progression-free survival for patients reached 24.8 months, with the overall survival rate at 36 months at 90.1%, indicating robust long-term efficacy (FIG. 18). IgG antibodies specific to the SurVaxM peptide were detected in a significant proportion of the patients, suggesting strong immune activation (FIG. 19).

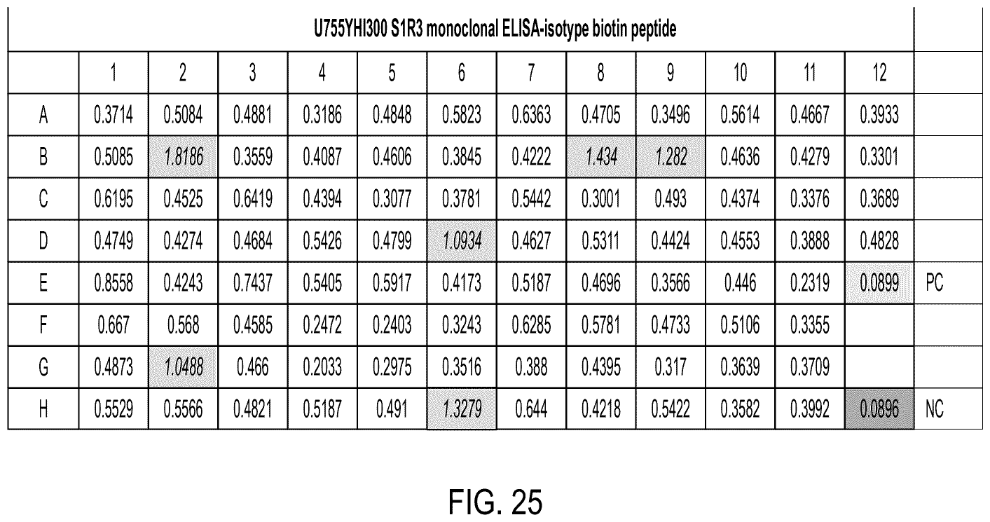

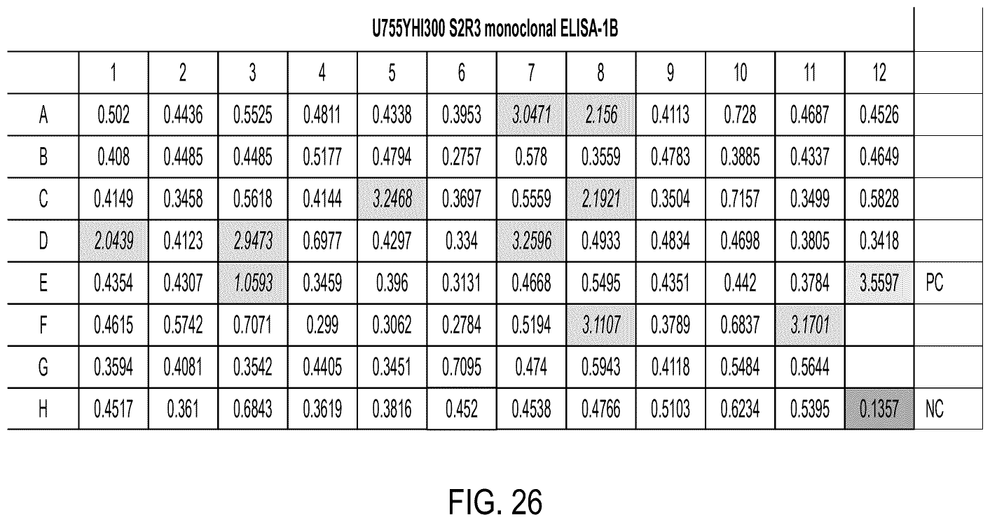

Further efficacy was observed in a Phase I study of SurVaxM combined with Octreotide for treating patients with metastatic NETs. This regimen proved beneficial in extending median progress-free survival to 10.9 months, an improvement over the typical outcomes with standard therapies (FIG. 20). The response to SurVaxM in NET patients, highlighted by the immunogenicity data in FIG. 21, confirms the potential of this vaccine to induce a strong and durable immune response against survivin-expressing tumor cells.

### Example 2: Development of Survivin-Specific sdAbs

Example 1 described the role of survivin in autoimmune conditions, such as MG, and the targeting of survivin by traditional IgG antibody. The present example survivin-specific sdAb development. These sdAbs are pursued due to their inherent robustness and capacity to bind with high specificity to epitopes that conventional antibodies might not access effectively. The decision to explore sdAbs is motivated by their potential to provide targeted therapy with reduced immunogenicity and improved tissue penetration, qualities essential for the targeting of survivin-positive cells implicated in autoimmune pathologies. Investigators thus proceeded with the goal of constructing a naïve sdAb phage display library to target a survivin-derived antigen. The overarching goal was to identify lead sdAbs that exhibited high specificity and affinity towards the survivin-derived antigen. This process involved the construction and screening of a phage display library, followed by the selection of candidate sdAbs through successive rounds of biopanning and detailed monoclonal ELISA screening and assessment.

A naïve sdAb phage display library was prepared from PBMCs of non-immunized camelids. The isolation of PBMCs was followed by RNA extraction and reverse transcription to obtain cDNA, which encodes the variable domains of heavy-chain-only antibodies (VHH). These sequences were then cloned into phage display vectors, yielding a diverse repertoire of sdAbs without predispositions towards specific antigens, facilitating unbiased biopanning. The library was stratified into discrete sub-libraries, or pools, termed S1, S2, and S3, each representing a unique segment of the library to enhance the efficiency of the subsequent screening process.

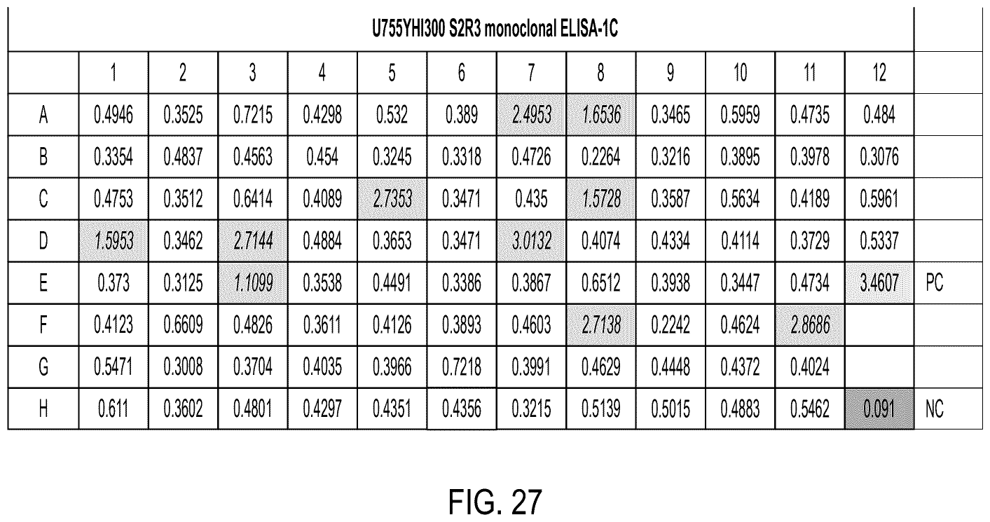

The naïve library was subjected to biopanning against two survivin-derived peptides over three rounds, as outlined in FIG. 22. Two peptides, each synthesized with a high degree of purity exceeding 90.0%, were used to attract and bind with the specific antibodies being screened in the process. The first peptide, denoted “1”, was a biotinylated mutated version of the survivin-derived antigen. The second peptide, denoted “1C”, was a biotinylated wildtype version of the same peptide. In the initial round, plates were coated with a 100 nM concentration of biotinylated wild-type peptide per pool and subjected to eight washes with 0.05% PBST, followed by three washes with PBS, ensuring the removal of non-specific binders. The subsequent round used a biotinylated mutated peptide at a reduced concentration of 50 nM per pool, where the number of washes was escalated to ten with 0.05% PBST and five with PBS to further refine the specificity of the binders. The final round mirrored the second in terms of concentration and washing rigor but reverted to using the biotinylated wild-type peptide, thereby reinforcing the selection for binders that retained high specificity for the original antigenic form. Each round was denoted by an “R” suffix indicating the round number (e.g., S1R1 for round 1 of pool S1). This series of progressively stringent rounds aimed to amplify the retention of phages that presented sdAbs with the strongest affinity for the target epitopes.

The potential lead sdAbs resulting from this panning protocol were then subjected to an ELISA protocol to evaluate binding affinities to both mutated (1B) and wild-type (1C) peptide antigens. In this protocol, plates were first coated with an antigen concentration of 1 ug/ml in CBS. This was followed by washing the plate twice using 0.05% PBST to remove any unbound antigen. To block non-specific interactions, the plate was then treated with 3% MPBS and incubated at 37° C. for 1 hour. After this blocking step, the plate was again washed once with 0.05% PBST. Subsequently, the plate received a mixture of 50 ul phage supernatant and 50 ul 0.05% PBST and was kept at room temperature for a 2 hour incubation period. Afterwards, a solution of 1 ug/ml of benchmark was added in a 100 ul volume, followed by incubation at room temperature for an additional 2 hours. To detect bound phages, an anti-M13 antibody coupled to HRP was added at a ratio of 1:40000 and incubated at 37° C. for 30 minutes. This series of steps ensured a consistent evaluation of binding affinities of all potential lead sdAbs.

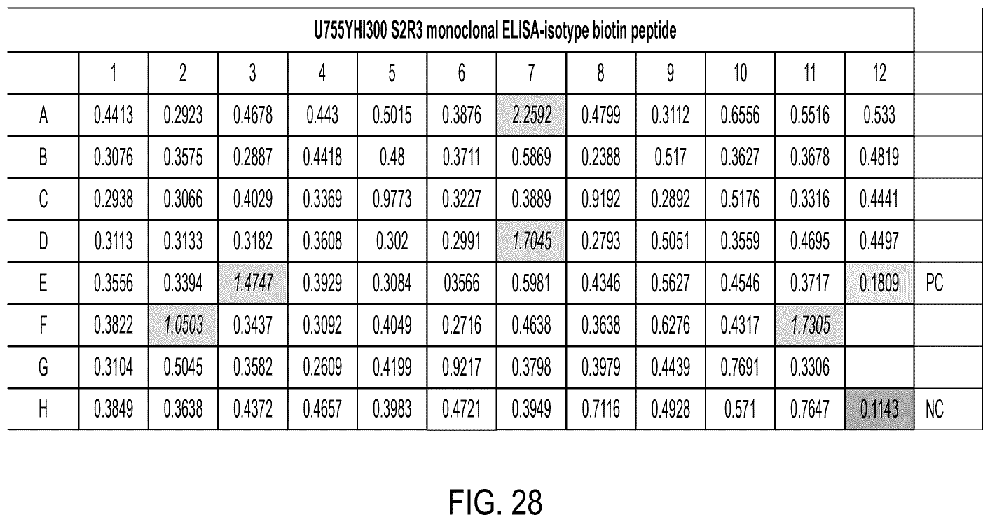

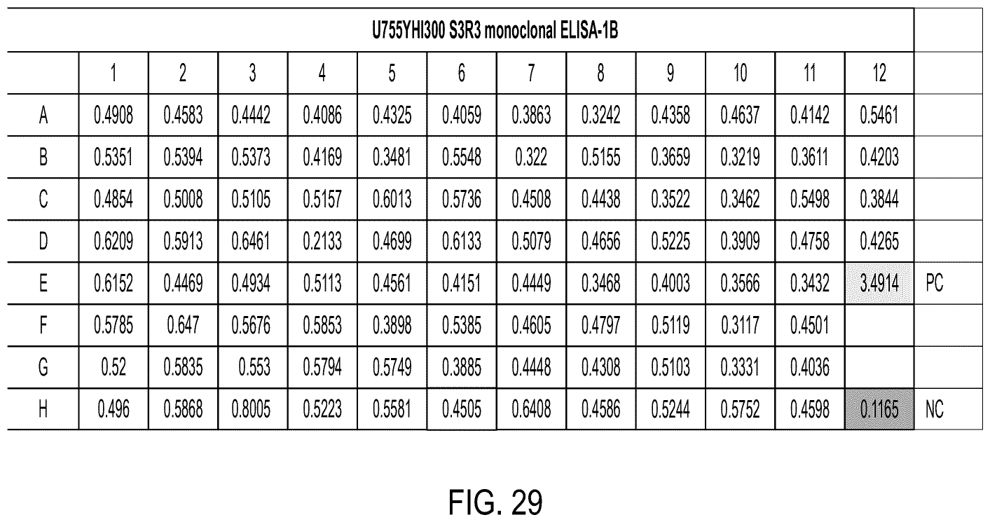

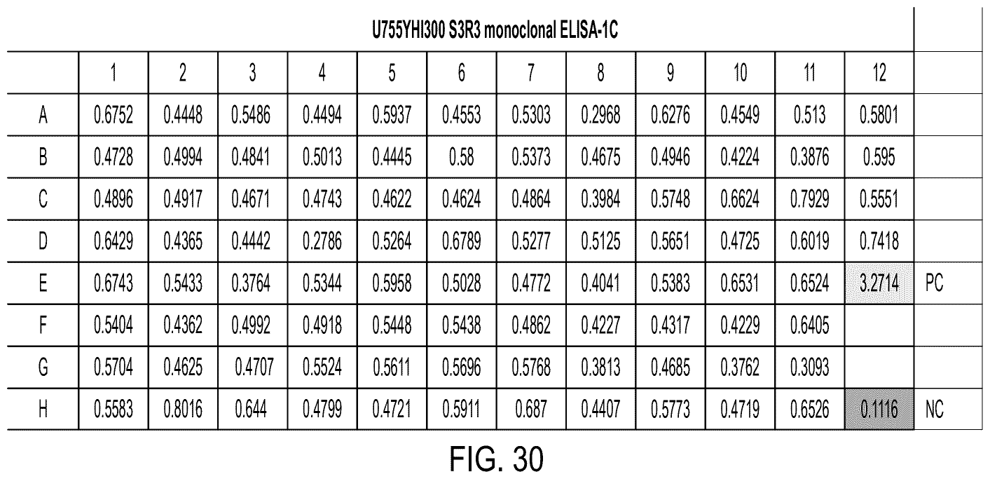

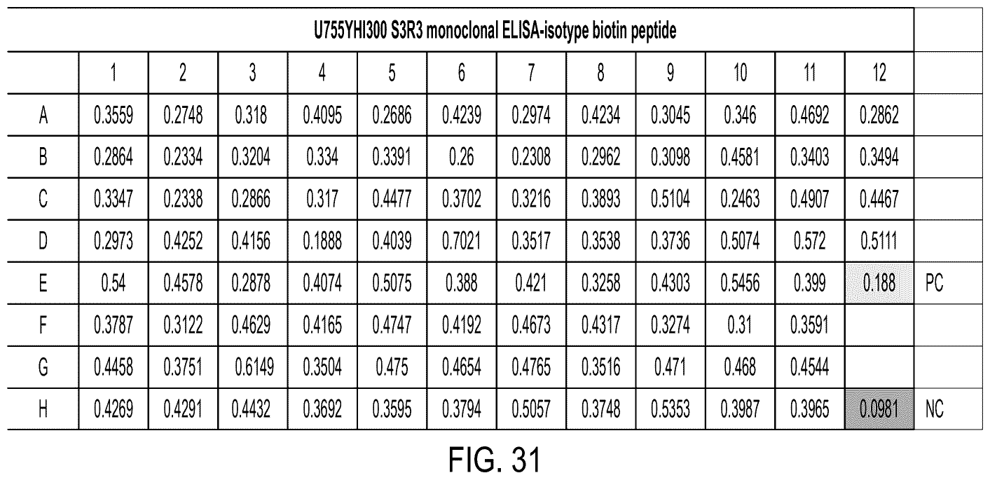

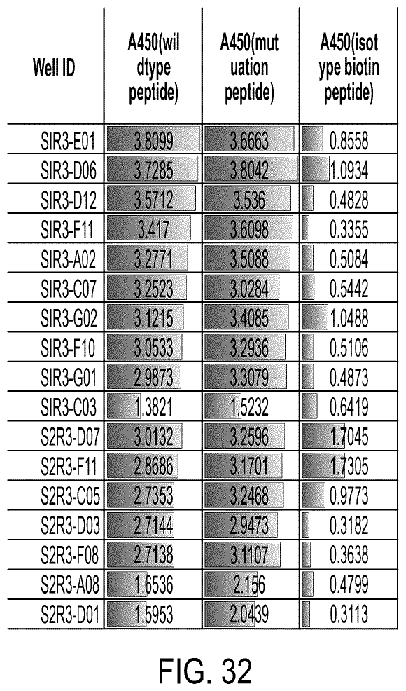

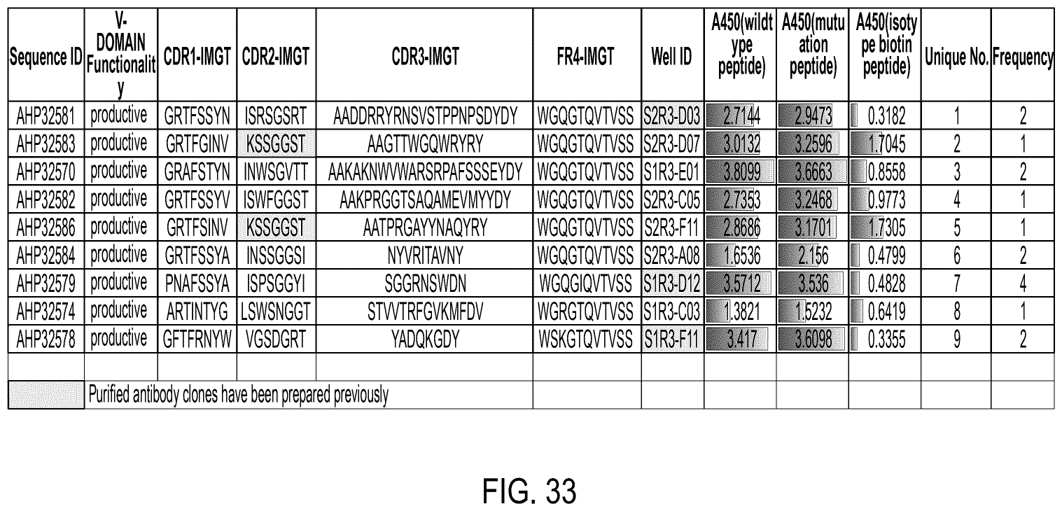

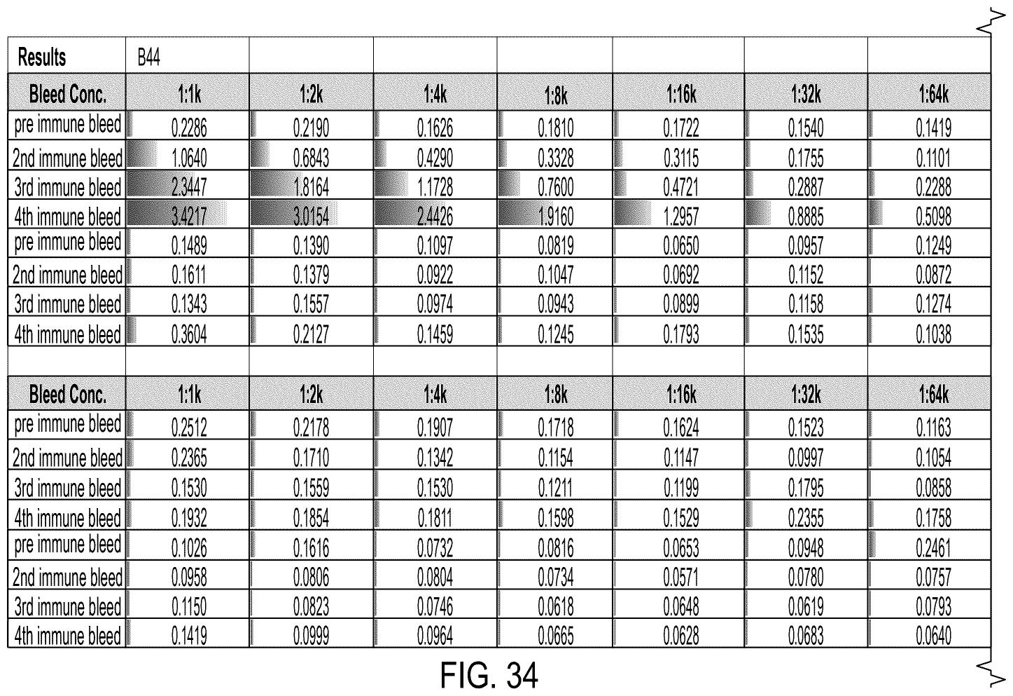

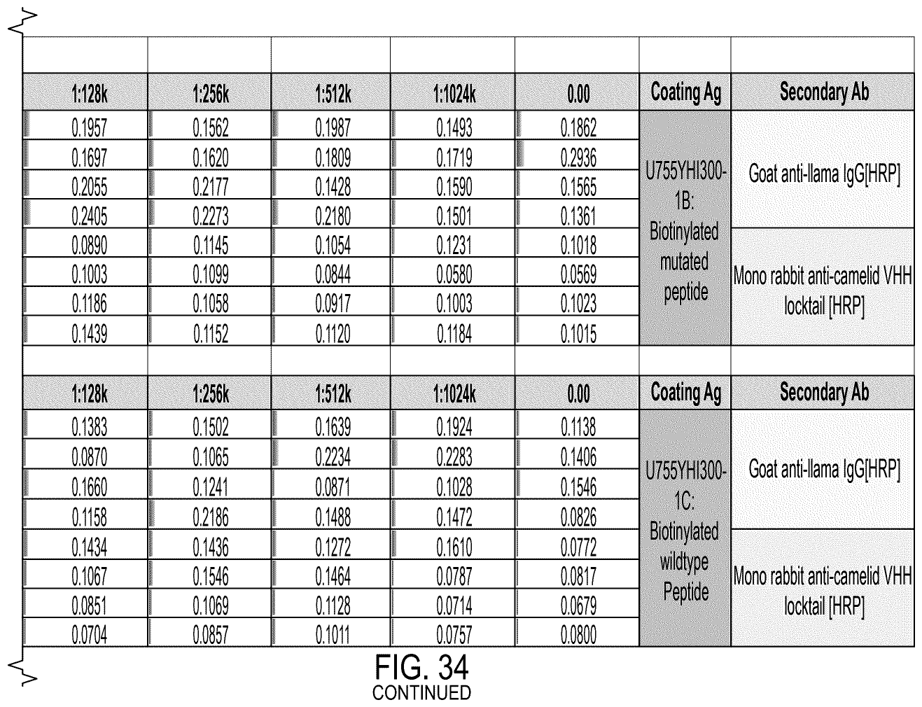

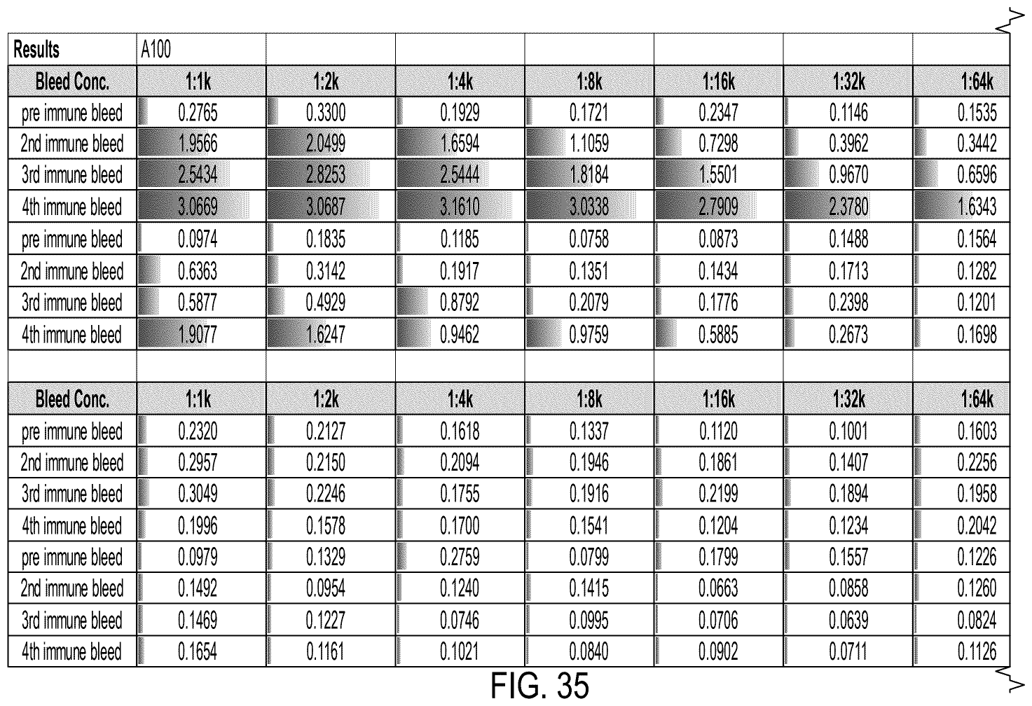

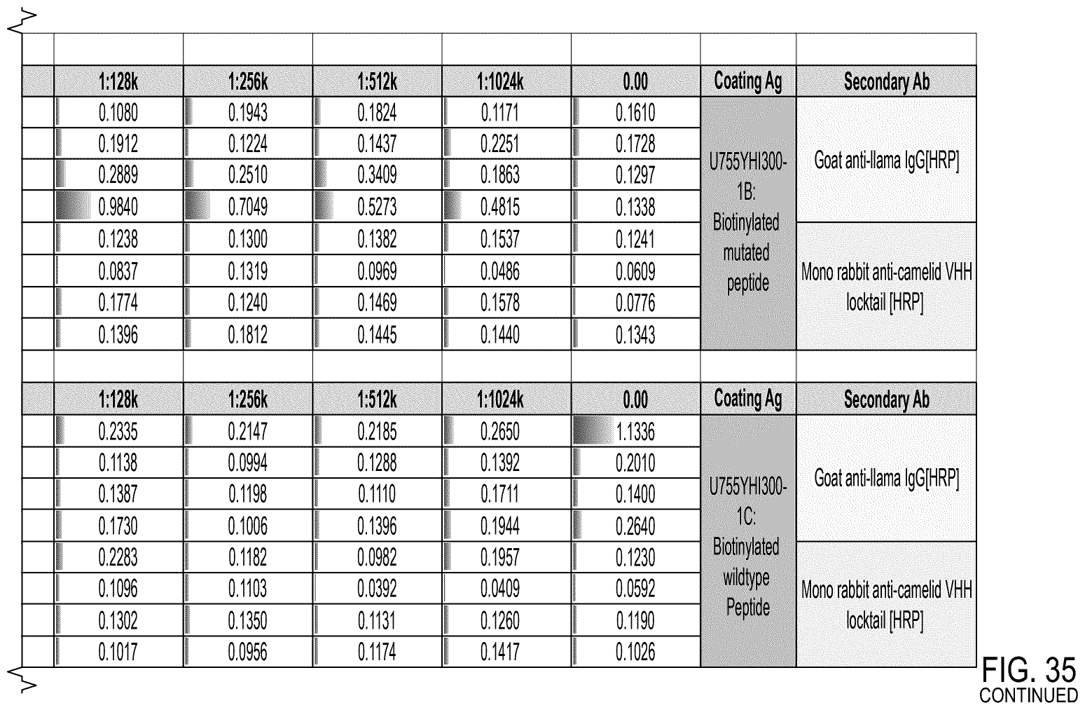

FIGS. 23-31 depict the resulting ELISA obtained after the third and final round of biopanning (R3), providing quantitative assessments of the binding affinities between the selected sdAbs and the survivin-derived peptides. Each figure corresponds to a specific pool and experimental condition. FIG. 23 depicts the ELISA data for pool S1 following the third round of biopanning, using peptide 1B. FIG. 24 depicts the data for pool S1 following the third round of biopanning, using peptide 1C. FIG. 25 depicts the control data for pool S1 using an isotype biotin peptide, serving as a benchmark to assess the specificity of the antibody-antigen interaction. FIG. 26 depicts the data for pool S2 using peptide 1B after the third round. FIG. 27 depicts the data for pool S2 using peptide 1C after the third round. FIG. 28 depicts the control data for pool S2 against the isotype biotin peptide. FIG. 29 depicts the data for pool S3 using peptide 1B after the third round. FIG. 30 depicts the data for pool S3 using peptide 1C after the third round. FIG. 31 depicts the control data for pool S3 against the isotype biotin peptide. FIG. 32 further quantifies the binding affinities, presenting ELISA absorbance readings at 450 nm for selected sdAb clones from the third round of biopanning. The data delineates the specific interaction levels of the clones with the wild-type and mutant survivin-derived peptides, compared against an isotype biotin peptide control, thus validating the specificity and potential efficacy of the selected antibody fragments.

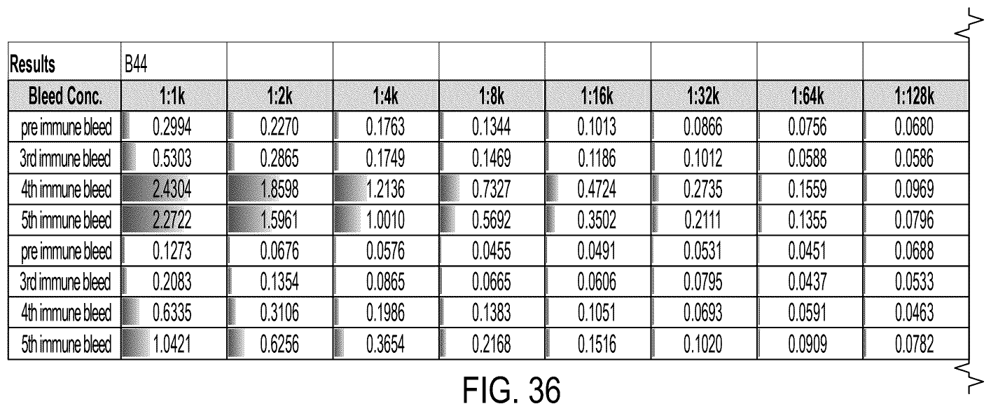

Applying a positive clone selection criterion—where a clone was considered positive if A450 readings for the wild-type (1C) or mutated (1B) peptides exceeded 1.0, and the A450 for the isotype control peptide was at least 0.8 units lower than for the specific peptides—a total of nine positive clones were identified, signifying the isolation of specific sdAbs with high affinity to the survivin-derived antigens. The selected clones were subjected to sequence alignment to ensure the uniqueness of each sdAb. FIG. 33 depicts the nucleotide and corresponding amino acid sequences of the complementarity-determining regions (CDRs) of the heavy chain variable domains (VHH) from the selected sdAbs.

In sum, the research efforts culminated in the successful identification of nine single-domain antibodies (sdAbs) with promising therapeutic potential, providing a foundation for the subsequent optimization.

### Example 3: Advancing sdAb Development Through Active Immunization

Leveraging the results presented in Example 2, the investigation transitioned to generating a sdAb phage display library derived from actively immunized alpacas, aiming to intensify the library's diversity and affinity for survivin-based antigens. This phase of the research was critical for augmenting the library with sdAbs of higher specificity and affinity. The inclusion of actively immunized alpacas served to simulate a more diverse immune response, thereby enriching the library with a wider array of potentially therapeutic sdAbs.

A. Generation and Screening of sdAb Library from Immunized Alpacas

Two alpacas, denoted B44 and A100, underwent a systematic immunization schedule with SurVaxM (SVN53-67/M57). The regimen started with an initial immunization followed by a series of booster shots. The doses were delivered at zero days, with subsequent boosts at two-week intervals. The immunogen used was the target protein at a quantity of 0.5 mg for the primary immunization, followed by 0.25 mg for the subsequent boosts. Blood samples were collected from the animals at pre-defined stages: before the immunization and after each immunization round. These samples were used to assess the immune response dynamics through ELISA and flow cytometry, providing data on the serum's antigen-specific activity. The test bleeds, taken at days 21, 35, 49, and 63, served as interim checkpoints for monitoring the immune response's maturation.

ELISA procedures were performed on the test bleeds to closely monitor the immune response elicited by the immunization protocol. The aim was to discern the most potent immune responders for the purposes of constructing a high-quality sdAb library. The ELISA procedures started with the coating of wells with 1 ug/ml of antigens, specifically peptides 1B, a biotinylated mutated survivin-derived peptide, and 1C, the biotinylated wild-type counterpart. The plate was then washed twice using 0.05% PBST to eliminate unwarranted clinging of unbound substances. The plate was blocked with 3% MPBS and then maintained at a temperature of 37° C. for a period of 60 minutes. A wash was performed again using 0.05% PBST. The immune bleed was introduced at initial concentration of 1:1000, applied in a 2-fold gradient dilution in 0.05% PBST, and then incubated at 37° C. for 60 minutes. This was followed by two different detection methods: either 0.1 g/ml of Goat anti-llama IgG [HRP] or 0.1 g/ml of Mono rab Rabbit Anti-Camelid VHH Locktail [HRP] were added in each well, at a volume of 100 ul, followed by incubation at 37° C. for a duration of 30 minutes. These procedures aimed to capture the potential high affinity sdAbs from the immunized alpacas.

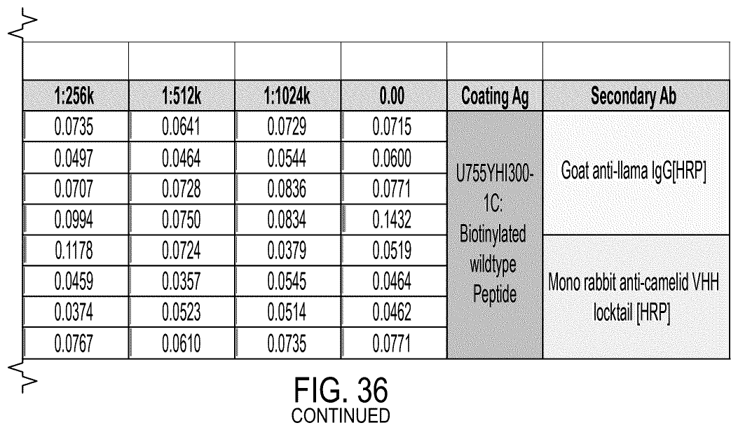

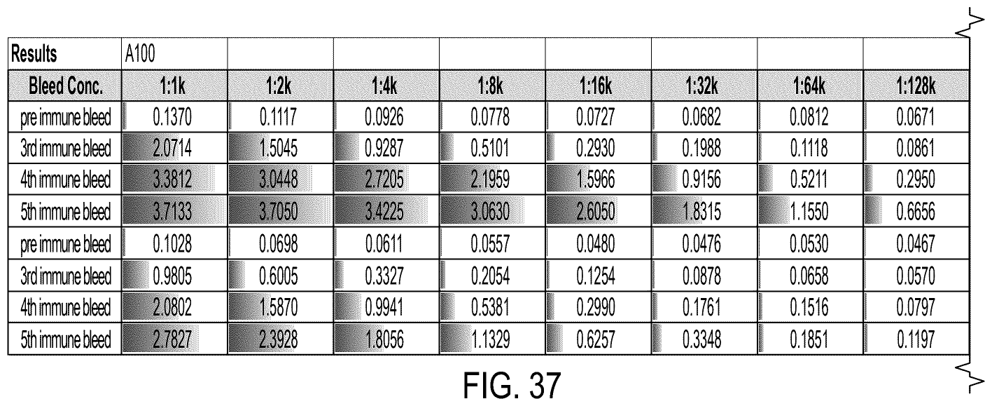

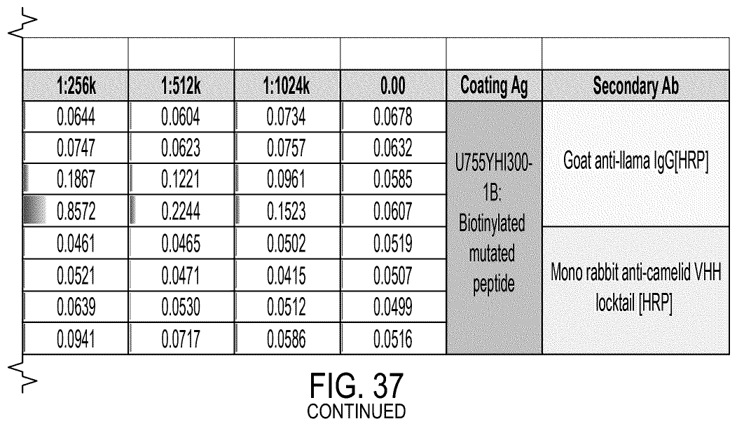

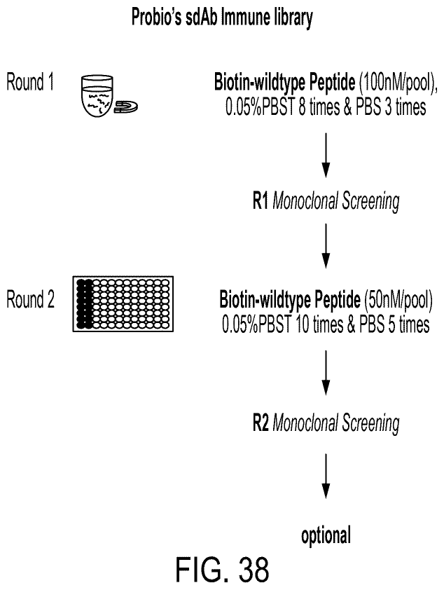

The resultant ELISA data, depicted in FIGS. 34-37, informed the curation of the sdAb library, ensuring the incorporation of antibodies stemming from the most efficacious immune responses. FIG. 34 depicts B44's 2^{nd} to 4^{th} immune bleed responses to peptides 1B and 1C, while FIG. 35 depicts A100's corresponding bleeds. FIG. 36 depicts the 3^{rd} to 5^{th} immune bleeds for B44 using peptide 1B, and FIG. 37 depicts similar bleeds from A100, also against peptide 1B. Collectively, these figures depict a progressive enhancement in antigen-specific activity across successive bleeds, demonstrating the alpacas' robust immunization-induced antibody responses. Peaks in absorbance values suggest successful induction of the targeted immune reaction, with higher titers observed in the later bleeds.

Upon the conclusion of the immunization schedule and the acquisition of blood samples, the construction of the sdAb library was carried out. Total RNA was isolated from the PBMCs of the alpacas which had been exposed to the immunization protocol. VHH genes, the hallmark of heavy-chain only antibodies present in camelids, were then amplified using tailored primers designed for this purpose. The resulting PCR products were purified to ensure the integrity of the genetic material for subsequent steps.

These VHH fragments were cloned into an M13 phage display vector, thus generating a phage display library with the desired capacity, which exceeded 5×10⁸. Quality control measures were applied to the library, revealing an insert rate surpassing 90%, which indicated a high probability of functional sdAb expression. Additionally, an in-frame rate of over 80% was confirmed, signifying that a substantial proportion of the sdAb sequences were correctly aligned for proper translation. The diversity of the library was also assessed, yielding a high percentage (95.83%) that demonstrated the library's varied repertoire. This phase validated the effectiveness of the immunization and the library's construction, setting the stage for the identification of specific binders.

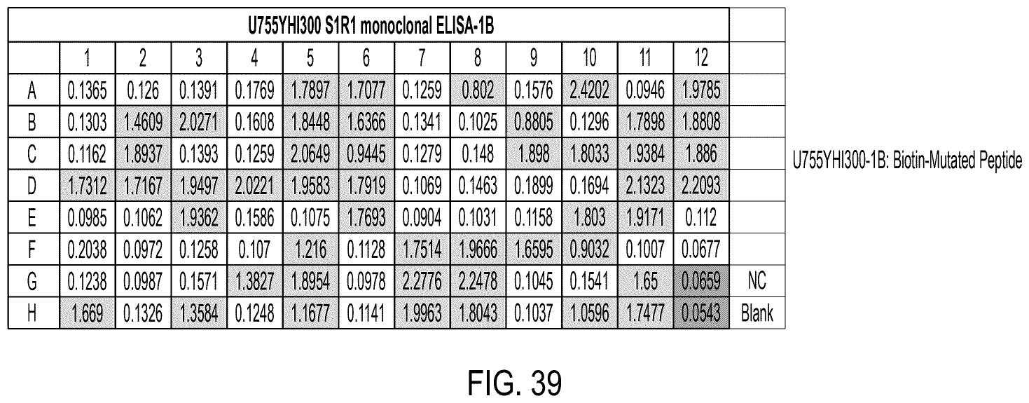

The panning process of the actively immunized sdAb library against the survivin-derived antigen was conducted over two rounds, as depicted in FIG. 38. Two peptides, each synthesized with a high degree of purity exceeding 90.0%, were used to attract and bind with the specific antibodies being screened in the process. The first peptide was a biotinylated mutated version of the survivin-derived antigen, denoted 1B. The second peptide was a biotinylated wildtype version of the same peptide, denoted 1C. In the initial round, plates were coated with a 100 nM concentration of biotinylated wild-type peptide per pool and subjected to eight washes with 0.05% PBST, followed by three washes with PBS, ensuring the removal of non-specific binders. The subsequent round used a biotinylated mutated peptide at a reduced concentration of 50 nM per pool, where the number of washes was escalated to ten with 0.05% PBST and five with PBS to further refine the specificity of the binders.

The potential lead sdAbs resulting from this panning protocol were then subjected to an ELISA protocol to evaluate binding affinities to both mutated (1B) and wild-type (1C) peptide antigens. In this protocol, plates were first coated with an antigen concentration of 1 ug/ml in CBS. This was followed by washing the plate twice using 0.05% PBST to remove any unbound antigen. To block non-specific interactions, the plate was then treated with 3% MPBS and incubated at 37° C. for 1 hour. After this blocking step, the plate was again washed once with 0.05% PBST. Subsequently, the plate received a mixture of 50 ul phage supernatant and 50 ul 0.05% PBST and was kept at room temperature for a 2 hour incubation period. Afterwards, a solution of 1 ug/ml of benchmark was added in a 100 ul volume, followed by incubation at room temperature for an additional 2 hours. To detect bound phages, an anti-M13 antibody coupled to HRP was added at a ratio of 1:40000 and incubated at 37° C. for 30 minutes. This series of steps ensured a consistent evaluation of binding affinities of all potential lead sdAbs.

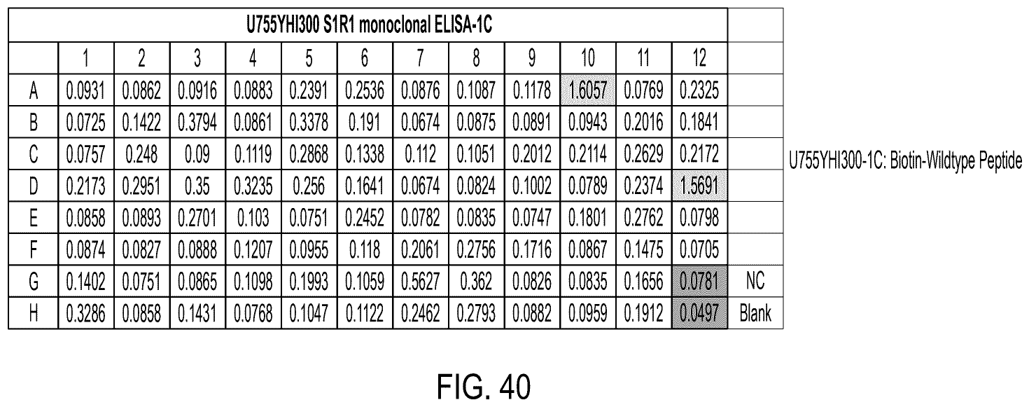

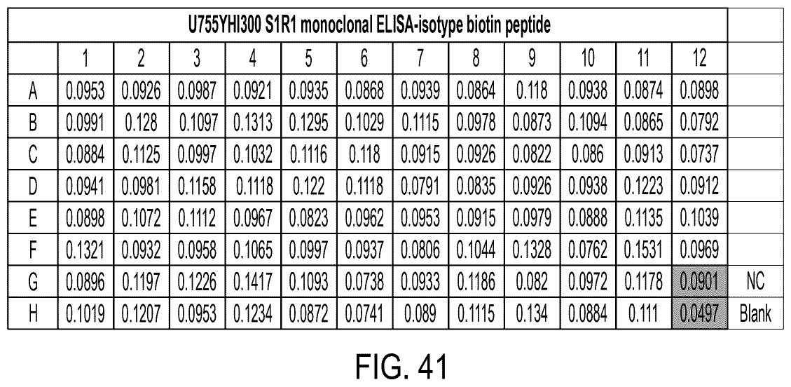

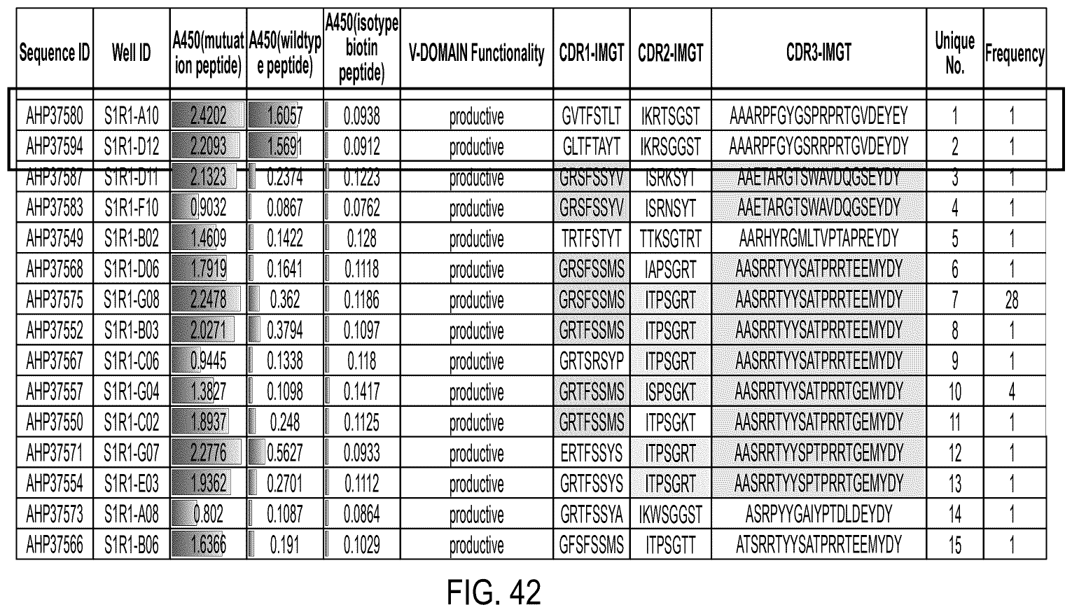

FIGS. 39-41 depict the resulting ELISAs obtained after the first round of biopanning (R1). FIG. 39 depicts the ELISA data for pool S1 following the first round of biopanning using peptide 1B. FIG. 40 depicts the ELISA data for pool S1 following the first round of biopanning using peptide 1C. FIG. 41 depicts the control data for pool S1 using an isotype biotin peptide, serving as a benchmark to assess the specificity of the antibody-antigen interaction.

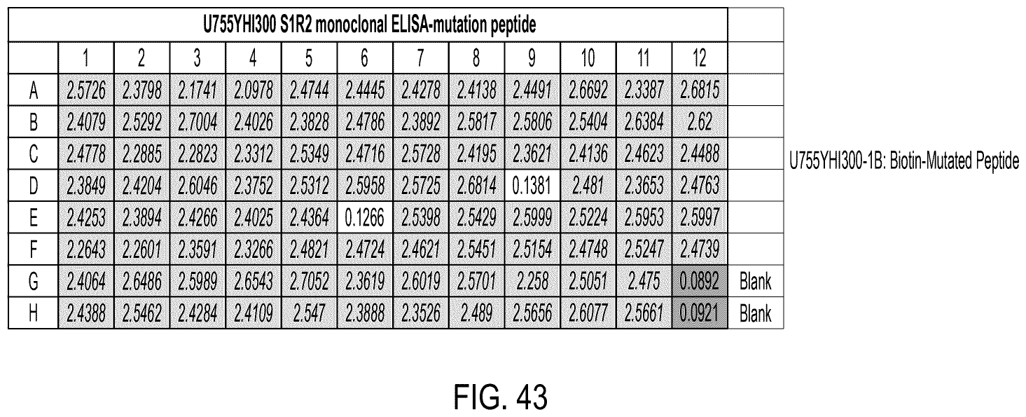

Using the same positive clone selection criterion as described in Example 4, a total of two positive clones were identified. The selected clones were subjected to sequence alignment to ensure the uniqueness of each sdAb. FIG. 42 depicts the nucleotide and corresponding amino acid sequences of the complementarity-determining regions (CDRs) of the heavy chain variable domains (VHH) from the selected sdAbs.

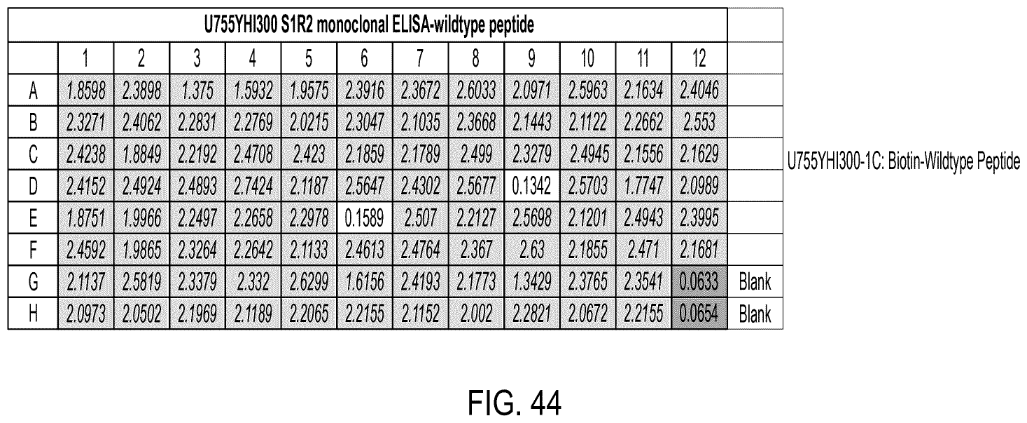

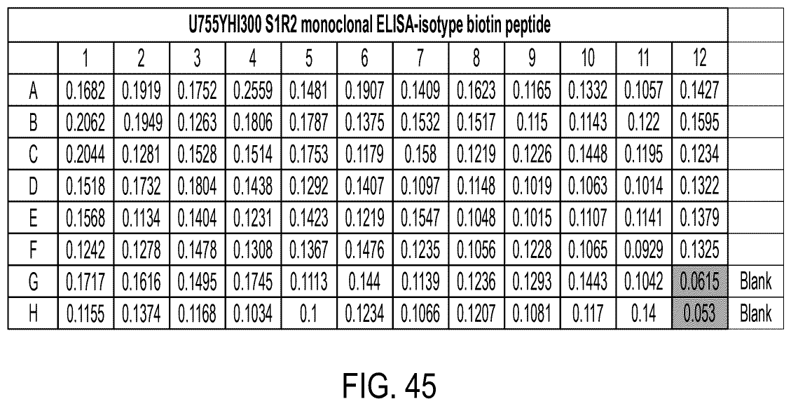

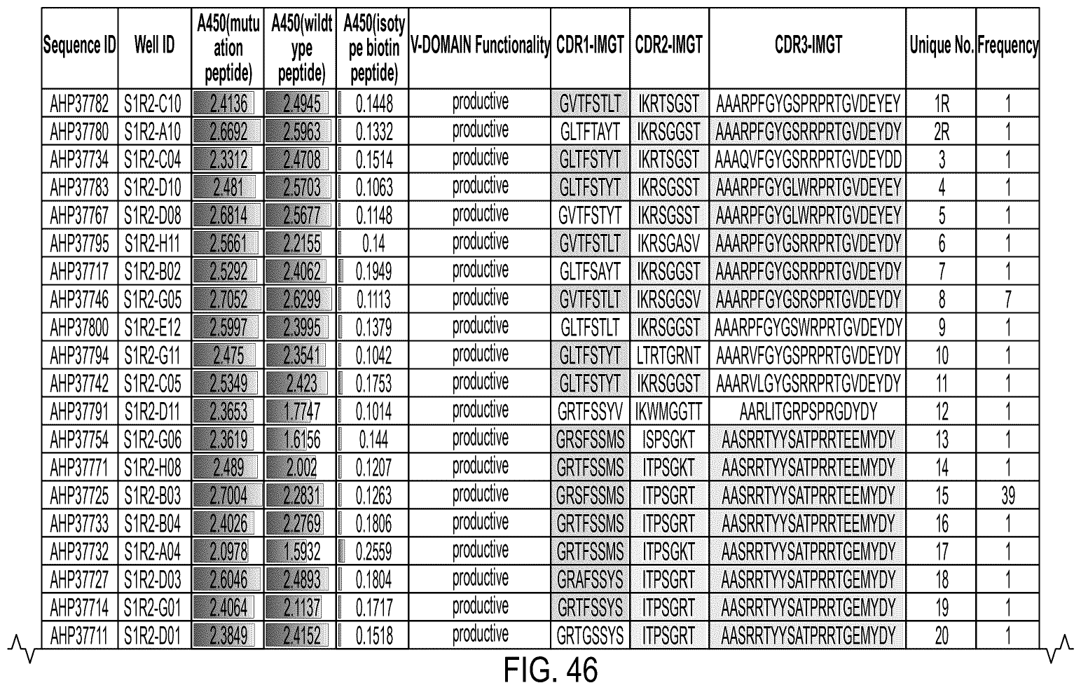

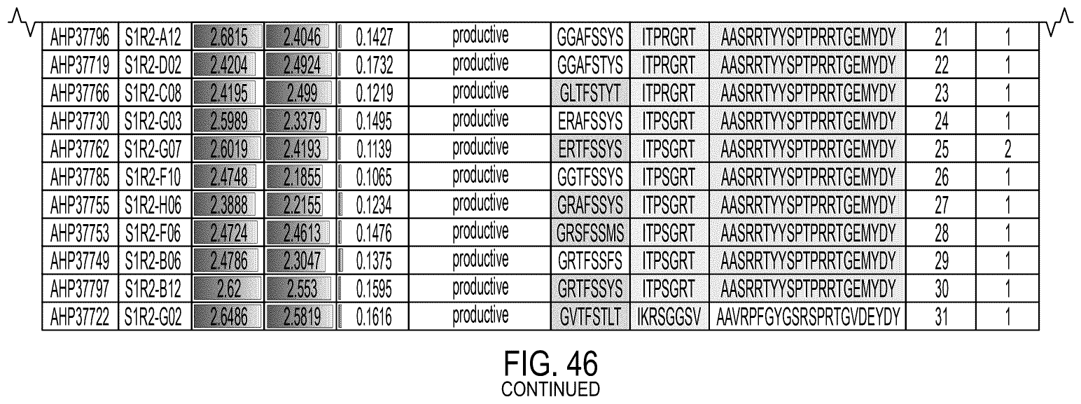

FIGS. 43-45 depict the resulting ELISAs obtained after the second round of biopanning (R2). The second round's ELISA results highlight a significant increase in binding activity with a total of 92 clones showing affinity to both the wildtype and mutated peptides. Following this, DNA sequencing results, as depicted in FIG. 46, identified 31 unique clones, which, when compared to the first round, accounted for an additional 29 unique clones. Collectively, these results quantify the reactivity of over 31 unique clones, each reflecting a high degree of specificity towards the survivin peptides.

B. Characterization and Selection of Lead sdAb Clones

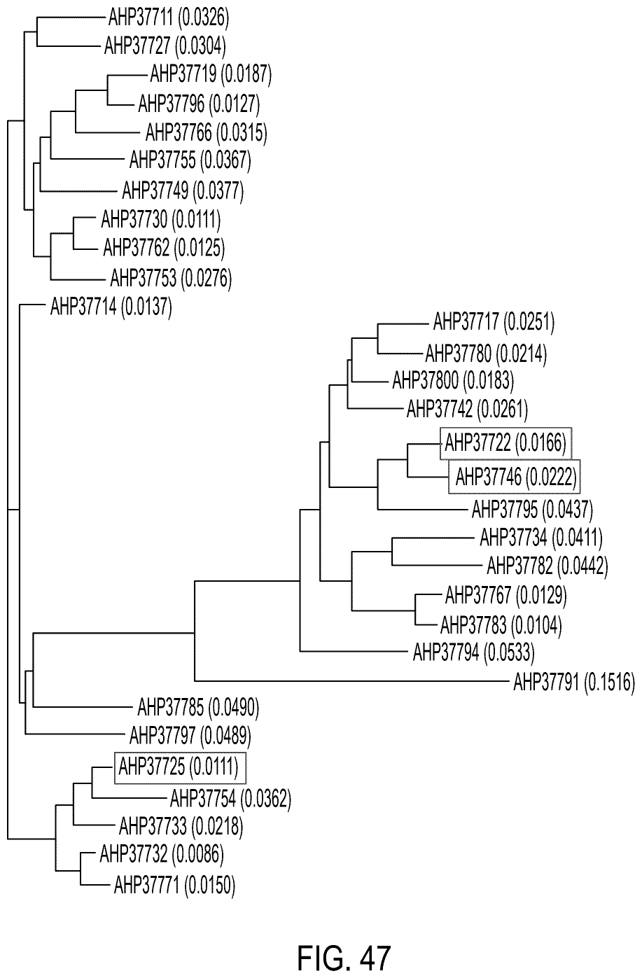

The focus then shifted to the characterization of selected lead candidates based on their binding efficacy and solubility. To this end, three clones—designated as Clone 1 (AHP37746), Clone 7 (AHP37725), and Clone 9 (AHP37722)—were selected for lead characterization based upon binding characteristics and solubility analysis, as depicted in FIG. 47. These clones underwent production in mammalian cell culture using ExpiCHO-S cells and an expression vector pcDNA3.4. A one-step purification process facilitated by Monofinity A Beads followed, yielding antibodies that were engineered to conjugate sdAb chains onto human IgG1-Fc backbone fragments. This engineering mimicry aligns the sdAb fragments with the structural integrity of full-size antibodies, specifically those akin to to original MV2C2 murine and humanized versions.

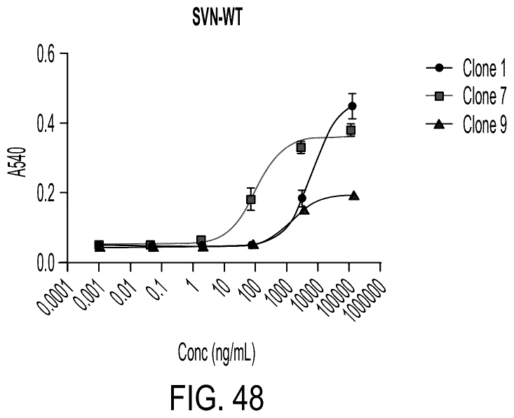

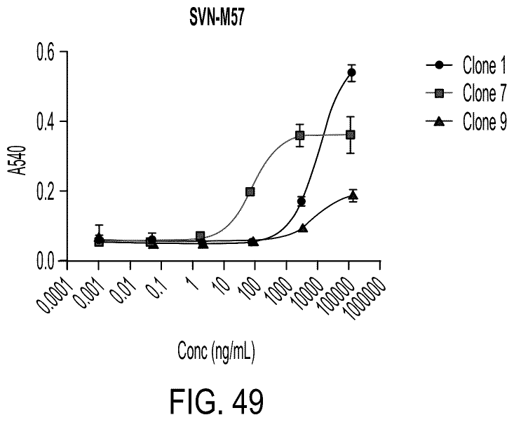

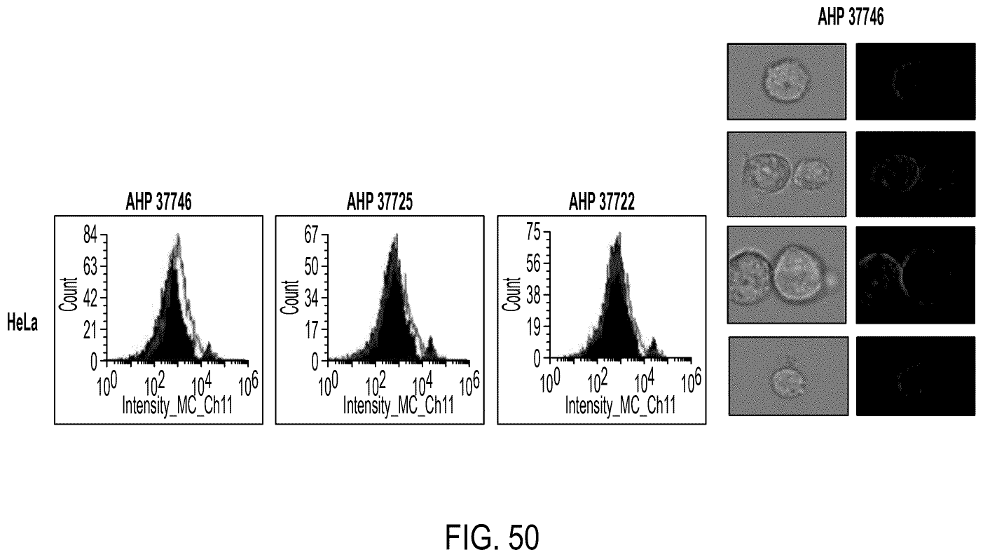

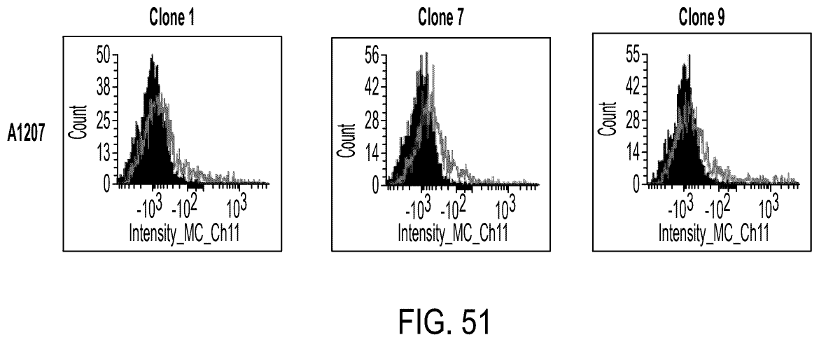

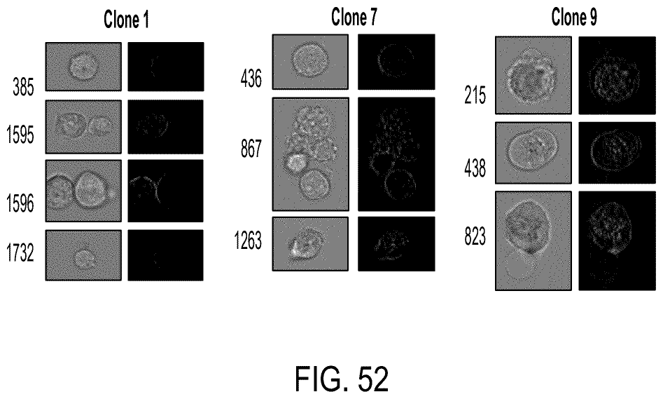

These selected sdAb clones were further analyzed through ELISA, assessing their reactivity against two forms of survivin: the wild-type SVN (WT-SVN) and the mutant M57-SVN. As depicted in FIGS. 48-49, the ELISA results revealed a distinct hierarchy in binding affinity, with Clone 1 showing a pronounced ability to bind to survivin when presented on the cell surface. This was validated through a flow cytometry method, which analyzed the binding of sdAb Clone 1 to non-permeabilized HeLa cells, as depicted in FIG. 50. The superior binding performance of Clone 1 implies a noteworthy potential for targeting survivin-overexpressing cells. This evaluation continued with the application of flow cytometry to assess the binding of sdAb clones to A1207 cells, again without permeabilization. The data, depicted in FIGS. 51-52, demonstrates the differential binding capacities across the clones. The visualization of this binding via fluorescent tagging in conjunction with bright field imaging allows for a granular assessment of the sdAbs' interactions with the cell surface.

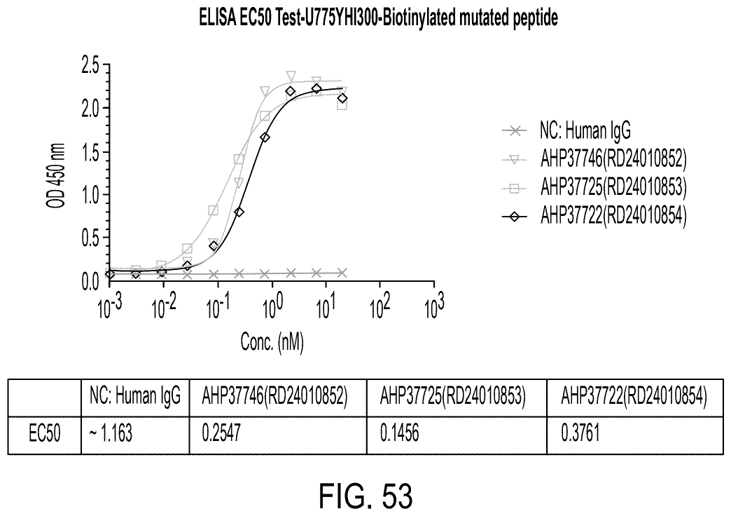

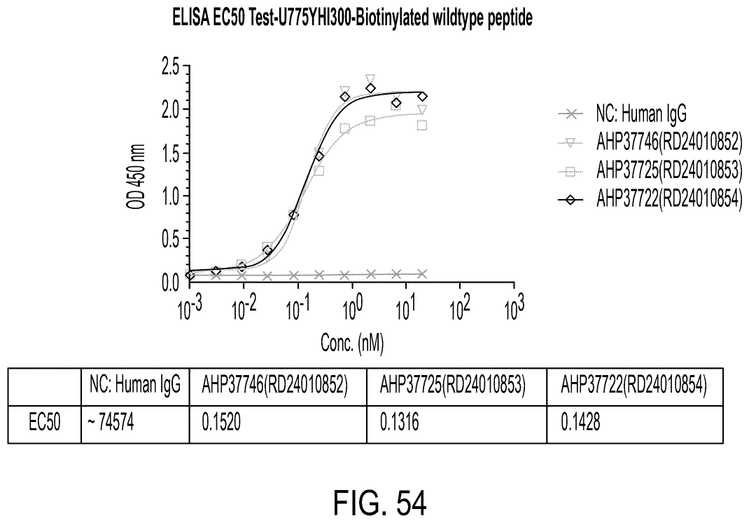

Further elucidating the specificity and affinity of the sdAb clones, FIGS. 53-54 depict the half maximal effective concentration (EC50) values from ELISA testing, reflecting the binding potency of the clones against both a biotinylated mutated peptide and a biotinylated wildtype peptide of survivin. FIG. 53 presents the data for the mutated peptide, illustrating a bell-shaped dose-response curve, characteristic of antigen-antibody interactions. Clone 1 displays an EC50 value of approximately 0.2547 nM, indicating a moderately high affinity binding. Clone 7 and Clone 9 display EC50 values of 0.1456 nM and 0.3761 nM, respectively. The negative control, human IgG, displays no appreciable binding as expected, validating the specificity of the sdAb clones. Similarly, FIG. 54 depicts the reactivity of the clones against the wildtype peptide. Here, the EC50 value for Clone 1 further corroborates its high affinity with a value of approximately 0.1520 nM, reinforcing the clone's potential for further development. The close EC50 values among the clones suggest a competitive binding scenario to the wildtype antigen, with all clones manifesting substantial affinity when compared to the markedly higher and non-specific EC50 value of the negative control, human IgG, which further affirms the assay's specificity.

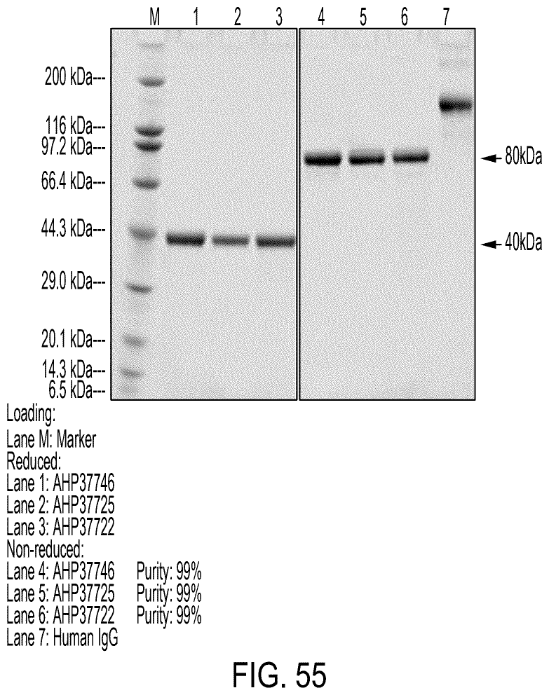

In terms of purity and molecular integrity, FIG. 55 depicts the results from SDS-PAGE analysis. The lanes designated for the sdAb clones, both under reducing and non-reducing conditions, exhibit a single band corresponding to the expected molecular weight, thereby confirming the monomeric nature and the high purity (>99%) of the sdAbs. The homogeneity of these bands, particularly when contrasted with the molecular weight marker, substantiates the successful expression and purification protocol. The control human IgG, is depicted under non-reducing conditions, showing the characteristic bands corresponding to the IgG heavy and light chains.

Collectively, these results demonstrate antibodies with high specificity and affinity, while maintaining structural integrity. Such findings are pivotal in transitioning these sdAb candidates from benchtop discovery to therapeutic entities.

In sum, the progression from initial immunization, through the biopanning of a diverse sdAb library, to the strategic selection and detailed characterization of lead clones, encapsulates a comprehensive approach to sdAb development. The effects have materialized in the identification of these three clones as outstanding candidates due to their binding attributes.

SEQUENCES

1. Antigen-Binding Fragments
SEQ ID NO: 1-Anti-survivin binding fragment, VHH1 CDR1; Anti-survivin
binding fragment, VHH6 CDR1; Anti-survivin binding fragment, VHH7 CDR1; Anti-
survivin binding fragment, VHH8 CDR1; Anti-survivin binding fragment, VHH31
CDR1
GVTFSTLT
SEQ ID NO: 2-Anti-survivin binding fragment, VHH1 CDR2; Anti-survivin
binding fragment, VHH3 CDR2
IKRTSGST
SEQ ID NO: 3-Anti-survivin binding fragment, VHH1 CDR3
AAARPFGYGSPRPRTGVDEYEY
SEQ ID NO: 4-Anti-survivin binding fragment, VHH1
EVQLVESGGGLVQAGGSLGLSCAASGVTESTLTMAWFRQASGKEREFVAAIKRTSG
STHYTDSVKGRFTISRDNAKNTVYLQMNSLKPEDTAVYYCAAARPFGYGSPRPRTG
VDEYEYWGQGTQVTVSS
SEQ ID NO: 5-Anti-survivin binding fragment, VHH2 CDR1
GLTFTAYT
SEQ ID NO: 6-Anti-survivin binding fragment, VHH2 CDR2; Anti-survivin
binding fragment, VHH7 CDR2; Anti-survivin binding fragment, VHH9 CDR2; Anti-
survivin binding fragment, VHH11 CDR2
IKRSGGST
SEQ ID NO: 7-Anti-survivin binding fragment, VHH2 CDR3; Anti-survivin
binding fragment, VHH6 CDR3; Anti-survivin binding fragment, VHH7 CDR3
AAARPFGYGSRRPRTGVDEYDY
SEQ ID NO: 8-Anti-survivin binding fragment, VHH2
DVQLVESGGGLVQAGGSLRVSCAASGLTFTAYTMGWFRQAPGKEREFVAVIKRSGG
STYYADSVKGRFTISRDNAKNTVYLQMNSLKSEDTAVYYCAAARPFGYGSRRPRTG
VDEYDYWGQGTQVTVSS
SEQ ID NO: 9-Anti-survivin binding fragment, VHH3 CDR1; Anti-survivin
binding fragment, VHH4 CDR1; Anti-survivin binding fragment, VHH10 CDR1; Anti-
survivin binding fragment, VHH11 CDR1; Anti-survivin binding fragment, VHH23
CDR1
GLTFSTYT
SEQ ID NO: 10-Anti-survivin binding fragment, VHH3 CDR3
AAAQVFGYGSRRPRTGVDEYDD
SEQ ID NO: 11-Anti-survivin binding fragment, VHH3
QVQLVESGGGLVQAGGSLGLSCAASGLTFSTYTMAWFRQAPGKEREFVAAIKRTSG
STYYTDSVKGRFTISRDNAKNTVYLQMNSLKPADTAVYYCAAAQVFGYGSRRPRTG
VDEYDDWGQGTQVTVSS
SEQ ID NO: 12-Anti-survivin binding fragment, VHH4 CDR2; Anti-survivin
binding fragment, VHH5 CDR2
IKRSGSST
SEQ ID NO: 13-Anti-survivin binding fragment, VHH4 CDR3; Anti-survivin
binding fragment, VHH5 CDR3
AAARPFGYGLWRPRTGVDEYEY
SEQ ID NO: 14-Anti-survivin binding fragment, VHH4
AVQLVDSGGGLVQPGGSLRLSCAASGLTFSTYTMAWFRQAPGKEREFVAAIKRSGS
STYYPDSVKGRFTISRDNAKNTVYLQMNSLKPADTAVYYCAAARPFGYGLWRPRTG
VDEYEYWGQGTQVTVSS
SEQ ID NO: 15-Anti-survivin binding fragment, VHH5 CDR1
GVTFSTYT
SEQ ID NO: 16-Anti-survivin binding fragment, VHH5
AVQLVDSGGGLVQAGGSLGLSCAASGVTFSTYTMA WFRQAPGKEREFVAAIKRSGS
STYYPDSVKGRFTISRDNAKNTVYLQMNSLKPADTAVYYCAAARPFGYGLWRPRTG
VDEYEYWGQGTQVTVSS
SEQ ID NO: 17-Anti-survivin binding fragment, VHH6 CDR2
IKRSGASV
SEQ ID NO: 18-Anti-survivin binding fragment, VHH6
DVQLVESGGGLVQAGGSLRLSCAASGVTFSTLTMGWFRQTPGKERVFVAAIKRSGA
SVYYADSVKGRFTISRDNAENTVYLQMISLKPEDTAVYYCAAARPFGYGSRRPRTG
VDEYDYWGQGTQVTVSS
SEQ ID NO: 19-Anti-survivin binding fragment, VHH7
DVQLVESGGGLVQAGGSLRVSCAASGLTFSAYTMGWFRQAPGKERDFVAAIKRSG
GSTFYADSVKGRFTISRDNAKNMVYLQMNSLKPEDTAVYYCAAARPFGYGSRRPRT
GVDEYDYWGQGTQVTVSS
SEQ ID NO: 20-Anti-survivin binding fragment, VHH8 CDR2; Anti-survivin
binding fragment, VHH31 CDR2
IKRSGGSV
SEQ ID NO: 21-Anti-survivin binding fragment, VHH8 CDR3
AAARPFGYGSRSPRTGVDEYDY
SEQ ID NO: 22-Anti-survivin binding fragment, VHH8
QVKLEESGGGLVQAGGSLRLSCTASGVTESTLTMGWFRQAPGKERVFVAAIKRSGG
SVYYADSVKGRFTISRDNAKNTVYLQMNSLKPEDTAVYYCAAARPFGYGSRSPRTG
VDEYDYWGQGTQVTVSS
SEQ ID NO: 23-Anti-survivin binding fragment, VHH9 CDR1
GLTFSTLT
SEQ ID NO: 24-Anti-survivin binding fragment, VHH9 CDR3
AAARPFGYGSWRPRTGVDEYDY
SEQ ID NO: 25-Anti-survivin binding fragment, VHH9
AVQLVESGGGLVQAGGSLRVSCAASGLTFSTLTMGWFRQAPGKEREFVAAIKRSGG
STYYADSVKGRFTISRDNAKNTVYLQMNSLKPEDTAVYYCAAARPFGYGSWRPRTG
VDEYDYWGQGTQVTVSS
SEQ ID NO: 26-Anti-survivin binding fragment, VHH10 CDR2
LTRTGRNT
SEQ ID NO: 27-Anti-survivin binding fragment, VHH10 CDR3
AAARVFGYGSPRPRTGVDEYDY
SEQ ID NO: 28-Anti-survivin binding fragment, VHH10
DVQLVESGGGLVQAGGSLRLSCAASGLTFSTYTMGWFRQAPGKEREIVAALTRTGR
NTYYADSVEGRFTISRDNAKNTVYLQMNSLKPEDTAVYYCAAARVFGYGSPRPRTG
VDEYDYWGQGTQVTVSS
SEQ ID NO: 29-Anti-survivin binding fragment, VHH11 CDR3
AAARVLGYGSRRPRTGVDEYDY
SEQ ID NO: 30-Anti-survivin binding fragment, VHH11
AVQLVDSGGGLVQAGGSLRVSCAASGLTESTYTMGWFRQAPGKERDFVAAIKRSG
GSTYYADSVKGRFTISRDNAKNTVYLQMNSLKPEDTAVYYCAAARVLGYGSRRPRT
GVDEYDYWGQGTQVTVSS
SEQ ID NO: 31-Anti-survivin binding fragment, VHH12 CDR1; Anti-survivin
binding fragment, VHH35 CDR1
GRTFSSYV
SEQ ID NO: 32-Anti-survivin binding fragment, VHH12 CDR2
IKWMGGTT
SEQ ID NO: 33-Anti-survivin binding fragment, VHH12 CDR3
AARLITGRPSPRGDYDY
SEQ ID NO: 34-Anti-survivin binding fragment, VHH12
QVQLVESGGGLVQAGGSLRLSCAASGRTFSSYVMAWFRQAPGEAREFIAAIKWMG
GTTSYAESVKGRFTISRDKAMNTVYLQMNSLKPEDTAVYYCAARLITGRPSPRGDY
DYWGQGTRVTVSA
SEQ ID NO: 35-Anti-survivin binding fragment, VHH13 CDR1; Anti-survivin
binding fragment, VHH15 CDR1; Anti-survivin binding fragment, VHH28 CDR1
GRSFSSMS
SEQ ID NO: 36-Anti-survivin binding fragment, VHH13 CDR2
ISPSGKT
SEQ ID NO: 37-Anti-survivin binding fragment, VHH13 CDR3; Anti-survivin
binding fragment, VHH14 CDR3; Anti-survivin binding fragment, VHH15 CDR3;
Anti-survivin binding fragment, VHH16 CDR3
AASRRTYYSATPRRTEEMYDY
SEQ ID NO: 38-Anti-survivin binding fragment, VHH13
QVKLEESGGGLVQAGDSLRLSCAASGRSFSSMSMGWFRQAPGTQREFVATISPSGKT
YYADSVKGRFTISRENAKDTVYLQMNSLKPEDTAVYYCAASRRTYYSATPRRTEEM
YDYWGQGTQVTVSS
SEQ ID NO: 39-Anti-survivin binding fragment, VHH14 CDR1; Anti-survivin
binding fragment, VHH16 CDR1; Anti-survivin binding fragment, VHH17 CDR1
GRTFSSMS
SEQ ID NO: 40-Anti-survivin binding fragment, VHH14 CDR2; Anti-survivin
binding fragment, VHH17 CDR2
ITPSGKT
SEQ ID NO: 41-Anti-survivin binding fragment, VHH14
AVQLVDSGGGLVQAGDSLRLSCAVSGRTFSSMSMGWFRQAPGKQREFVATITPSGK
TYYADSVKGRFTISRENAKDTVYLQMNSLKPEDTAVYYCAASRRTYYSATPRRTEE
MYDYWGQGTQVTVSS
SEQ ID NO: 42-Anti-survivin binding fragment, VHH15 CDR2; Anti-survivin
binding fragment, VHH16 CDR2; Anti-survivin binding fragment, VHH18 CDR2;
Anti-survivin binding fragment, VHH19 CDR2; Anti-survivin binding fragment,
VHH20 CDR2; Anti-survivin binding fragment, VHH24 CDR2; Anti-survivin binding
fragment, VHH25 CDR2; Anti-survivin binding fragment, VHH26 CDR2; Anti-
survivin binding fragment, VHH27 CDR2; Anti-survivin binding fragment, VHH28
CDR2; Anti-survivin binding fragment, VHH29 CDR2; Anti-survivin binding
fragment, VHH30 CDR2
ITPSGRT
SEQ ID NO: 43-Anti-survivin binding fragment, VHH15
AVQLVDSGGGLVQAGDSLRLSCAASGRSFSSMSMGWFRQAPGTQREFVATITPSGR
TYYADSVKGRFTISRENAKDTVYLQMNSLKPEDTAVYYCAASRRTYYSATPRRTEE
MYDYWGQGTQVTVSS
SEQ ID NO: 44-Anti-survivin binding fragment, VHH16
QVQLVESGGGLVQSGGSLRLPCAASGRTFSSMSMGWFRQAPGTQREFVATITPSGRT
YYADSVKGRFTISRENAKDTVYLQMNSLKPEDTAVYYCAASRRTYYSATPRRTEEM
YDYWGQGTQVTVSS
SEQ ID NO: 45-Anti-survivin binding fragment, VHH17 CDR3; Anti-survivin
binding fragment, VHH18 CDR3; Anti-survivin binding fragment, VHH19 CDR3;
Anti-survivin binding fragment, VHH20 CDR3
AASRRTYYSATPRRTGEMYDY
SEQ ID NO: 46-Anti-survivin binding fragment, VHH17
EVQLVESGGGLVQAGDSLRLSCAVSGRTFSSMSMGWFRQAPGKQREFVATITPSGK
TYYADSVKGRFTISRENAKDTVYLQMNSLKPEDTAVYYCAASRRTYYSATPRRTGE
MYDYWGQGTQVTVSS
SEQ ID NO: 47-Anti-survivin binding fragment, VHH18 CDR1; Anti-survivin
binding fragment, VHH27 CDR1
GRAFSSYS
SEQ ID NO: 48-Anti-survivin binding fragment, VHH18
AVQLVDSGGGLVQAGGSLRLSCATSGRAFSSYSMGWFRQAPGKEREFVATITPSGRT
HYADSVKGRFTISRENAKDTVYLQMNSLKPADTAVYYCAASRRTYYSATPRRTGEM
YDYWGQGTQVTVSS
SEQ ID NO: 49-Anti-survivin binding fragment, VHH19 CDR1; Anti-survivin
binding fragment, VHH30 CDR1
GRTFSSYS
SEQ ID NO: 50-Anti-survivin binding fragment, VHH19
AVQLVESGGGLVQPGGSLRLSCAVSGRTFSSYSMGWFRQAPGKEREFVATITPSGRT
YYADSVKGRFTISRENAKDTVYLQMNSLKPEDTAVYYCAASRRTYYSATPRRTGEM
YDYWGQGTQVTVSS
SEQ ID NO: 51-Anti-survivin binding fragment, VHH20 CDR1
GRTGSSYS
SEQ ID NO: 52-Anti-survivin binding fragment, VHH20
AVQLVDSGGGLVQAGGSLRVSCAASGRTGSSYSWGWFRQAPGKEREFVATITPSGR
TYYPDSVKGRFTISRENAKDTVYLQMNSLKPEDTAVYYCAASRRTYYSATPRRTGE
MYDYWGQGTQVTVSS
SEQ ID NO: 53-Anti-survivin binding fragment, VHH21 CDR1
GGAFSSYS
SEQ ID NO: 54-Anti-survivin binding fragment, VHH21 CDR2; Anti-survivin
binding fragment, VHH22 CDR2; Anti-survivin binding fragment, VHH23 CDR2
ITPRGRT
SEQ ID NO: 55-Anti-survivin binding fragment, VHH21 CDR3; Anti-survivin
binding fragment, VHH22 CDR3; Anti-survivin binding fragment, VHH23 CDR3;
Anti-survivin binding fragment, VHH24 CDR3; Anti-survivin binding fragment,
VHH25 CDR3; Anti-survivin binding fragment, VHH26 CDR3; Anti-survivin binding
fragment, VHH27 CDR3; Anti-survivin binding fragment, VHH28 CDR3; Anti-
survivin binding fragment, VHH29 CDR3; Anti-survivin binding fragment, VHH30
CDR3
AASRRTYYSPTPRRTGEMYDY
SEQ ID NO: 56-Anti-survivin binding fragment, VHH21
QVQLVESGGGLVQAGGSLRLSCAAAGGAFSSYSMGWFRQAPGKEREFVATITPRGR
TYSSDSVKGRFTISRENAEDTVYLQMNSLKPEDTAVYYCAASRRTYYSPTPRRTGEM
YDYWGQGTQVTVSS
SEQ ID NO: 57-Anti-survivin binding fragment, VHH22 CDR1
GGAFSTYS
SEQ ID NO: 58-Anti-survivin binding fragment, VHH22
QVQLVESGGGLVQPGDSLRLSCAAAGGAFSTYSMGWFRQAPGKEREFVATITPRGR
TYYSDSVKGRFTISRENAEDTVYLQMNSLKPEDTAVYYCAASRRTYYSPTPRRTGE
MYDYWGQGTQVTVSS
SEQ ID NO: 59-Anti-survivin binding fragment, VHH23
DVQLVESGGGLVQAGGSLRLSCAASGLTFSTYTMGWFRQAPGKEREFVATITPRGR
TYYSDSVKGRFTISRENAEDTVYLQMNSLKPEDTAAYYCAASRRTYYSPTPRRTGE
MYDYWGQGTQVTVSS
SEQ ID NO: 60-Anti-survivin binding fragment, VHH24 CDR1
ERAFSSYS
SEQ ID NO: 61-Anti-survivin binding fragment, VHH24
DVQLVESGGGLVQAGGSLRLSCAASERAFSSYSMGWFRQAPGKEREFVATITPSGRT
YYPDSVKGRFTISRENVKDTVYLQMNSLKPEDTAVYYCAASRRTYYSPTPRRTGEM
YDYWGQGTQVTVSS
SEQ ID NO: 62-Anti-survivin binding fragment, VHH25 CDR1
ERTFSSYS
SEQ ID NO: 63-Anti-survivin binding fragment, VHH25
EVQLVESGGGLVQSGGSLRLSCAASERTFSSYSMGWFRQAPGKEREFVATITPSGRT
YYPDSVKGRFTISRENVKDTVYLQMNSLKPEDTAVYYCAASRRTYYSPTPRRTGEM
YDYWGQGTQVTVSS
SEQ ID NO: 64-Anti-survivin binding fragment, VHH26 CDR1
GGTFSSYS
SEQ ID NO: 65-Anti-survivin binding fragment, VHH26
DVQLVESGGGLVQPGGSLRLSCAASGGTFSSYSMGWFRQAPGKEREFVATITPSGRT
YYADSMRGRFTISREGAKDTVYLQMNDLKPEDTAVYYCAASRRTYYSPTPRRTGEM
YDYWGQGTQVTVSS
SEQ ID NO: 66-Anti-survivin binding fragment, VHH27
EVQLVESGGGLVQTGGSLRLSCAVSGRAFSSYSMGWFRQAPGKERVFVATITPSGRT
YYSDSVKGRFTISRENAEDTVYLQMNSLKPEDTAVYYCAASRRTYYSPTPRRTGEM
YDYWGQGTHVTVSS
SEQ ID NO: 67-Anti-survivin binding fragment, VHH28
AVQLVDSGGGLVQAGDSLRLSCAASGRSFSSMSMGWFRQAPGKEREFVATITPSGR
TYYPDSVKGRFTISRENVKDTVYLQMNSLKPEDTAVYYCAASRRTYYSPTPRRTGE
MYDYWGQGTQVTVSS
SEQ ID NO: 68-Anti-survivin binding fragment, VHH29 CDR1
GRTFSSFS
SEQ ID NO: 69-Anti-survivin binding fragment, VHH29
AVQLVDSGGGLVQPGGSLRLSCAASGRTFSSFSMGWFRQAPGKEREFVATITPSGRT
YYSDSVKGRFTISRENPEATVYLQMNSLKPEDTAVYYCAASRRTYYSPTPRRTGEMY
DYWGQGTQVTVSS
SEQ ID NO: 70-Anti-survivin binding fragment, VHH30
QVKLEESGGGLVQAGGSLTLSCAVSGRTFSSYSMGWFRQAPGKEREFVATITPSGRT
YHANSVKGRFTISRENAKDTVYLQMNSLKPEDTAVYYCAASRRTYYSPTPRRTGEM
YDYWGQGTQVTVSS
SEQ ID NO: 71-Anti-survivin binding fragment, VHH31 CDR3
AAVRPFGYGSRSPRTGVDEYDY
SEQ ID NO: 72-Anti-survivin binding fragment, VHH31
AVQLVESGGGLVQAGESLRLSCTASGVTFSTLTMGWFRQAPGKERVFVAAIKRSGG
SVYYADSVKGRFTISRDNAKNTVYLQMNSLKPEDTAVYYCAAVRPFGYGSRSPRTG
VDEYDYWGQGTQVTVSS
SEQ ID NO: 73-Anti-survivin binding fragment, VHH32 CDR1
GRTFSSYN
SEQ ID NO: 74-Anti-survivin binding fragment, VHH32 CDR2
ISRSGSRT
SEQ ID NO: 75-Anti-survivin binding fragment, VHH32 CDR3
AADDRRYRNSVSTPPNPSDYDY
SEQ ID NO: 76-Anti-survivin binding fragment, VHH32
EVQLVESGGGLVQAGGSLRLSCAASGRTFSSYNMGWFRQAPGKEREYVAAISRSGS
RTYYLNSVKDRFTISRDNAKNTVYLQMNSLKPEDTAVYYCAADDRRYRNSVSTPPN
PSDYDYWGQGTQVTVSS
SEQ ID NO: 77-Anti-survivin binding fragment, VHH33 CDR1
GRTFGINV
SEQ ID NO: 78-Anti-survivin binding fragment, VHH33 CDR2; Anti-survivin
binding fragment, VHH36 CDR2
KSSGGST
SEQ ID NO: 79-Anti-survivin binding fragment, VHH33 CDR3
AAGTTWGQWRYRY
SEQ ID NO: 80-Anti-survivin binding fragment, VHH33
QVKLEESGGGLVQPGGSLSLSCAASGRTFGINVMGWYRQAPGKQRELVAAKSSGGS
TRYADSVKGRFTISRDNAKNTVYLQMNSLKPEDTAVYYCAAGTTWGQWRYRYWG
QGTQVTVSS
SEQ ID NO: 81-Anti-survivin binding fragment, VHH34 CDR1
GRAFSTYN
SEQ ID NO: 82-Anti-survivin binding fragment, VHH34 CDR2
INWSGVTT
SEQ ID NO: 83-Anti-survivin binding fragment, VHH34 CDR3
AAKAKNWVWARSRPAFSSSEYDY
SEQ ID NO: 84-Anti-survivin binding fragment, VHH34
AVQLVESGGGLVQAGGSLRLTCAASGRAFSTYNMAWFRQAPGKEREFVAAINWSG
VTTYYGDSVKGRFTISRDNAKNTVYLQMNSLKSEDTAVYYCAAKAKNWVWARSR
PAFSSSEYDYWGQGTQVTVSS
SEQ ID NO: 85-Anti-survivin binding fragment, VHH35 CDR2
ISWFGGST
SEQ ID NO: 86-Anti-survivin binding fragment, VHH35 CDR3
AAKPRGGTSAQAMEVMYYDY
SEQ ID NO: 87-Anti-survivin binding fragment, VHH35
QVKLEESGGGLVQAGGSLRLSCAASGRTFSSYVMGWFRQAPGKEREFVAAISWFGG
STRYADSVKGRFTISRDNAKNTVFLQMNTLKPEDTAVYYCAAKPRGGTSAQAMEV
MYYDYWGQGTQVTVSS
SEQ ID NO: 88-Anti-survivin binding fragment, VHH36 CDR1
GRTFSINV
SEQ ID NO: 89-Anti-survivin binding fragment, VHH36 CDR3
AATPRGAYYNAQYRY
SEQ ID NO: 90-Anti-survivin binding fragment, VHH36
QVQLQESGGGSVQPGGSLSLSCAASGRTFSINVMGWYRQAPGKQRELVAAKSSGGS
TRYADSVKGRFTISRDNAKNTVYLQMNNLKPEDTAVYYCAATPRGAYYNAQYRY
WGQGTQVTVSS
SEQ ID NO: 91-Anti-survivin binding fragment, VHH37 CDR1
GRTFSSYA
SEQ ID NO: 92-Anti-survivin binding fragment, VHH37 CDR2
INSSGGSI
SEQ ID NO: 93-Anti-survivin binding fragment, VHH37 CDR3
NYVRITAVNY
SEQ ID NO: 94-Anti-survivin binding fragment, VHH37
EVQLVESGGGLVQAGGSLRLSCAASGRTESSYAMGWFRQAPGKGRDFVGAINSSGG
SILYADSVKGRFTISRDNAKNTVFLQMNSLEPEDTAVYYCNYVRITAVNYWGQGTQ
VTVSS
SEQ ID NO: 95-Anti-survivin binding fragment, VHH38 CDR1
PNAFSSYA
SEQ ID NO: 96-Anti-survivin binding fragment, VHH38 CDR2
ISPSGGYI
SEQ ID NO: 97-Anti-survivin binding fragment, VHH38 CDR3
SGGRNSWDN
SEQ ID NO: 98-Anti-survivin binding fragment, VHH38
VQLVESGGGLVQTGGSLRLSCAASGPNAFSSYAMGWFRQRPGKEREFVAAISPSGG
YIQYEDSVKGRSTISRDNSKNMMYLQMVNLKPDDTAVYYCSGGRNSWDNWGQGIQ
VTVSS
SEQ ID NO: 99-Anti-survivin binding fragment, VHH39 CDR1
ARTINTYG
SEQ ID NO: 100-Anti-survivin binding fragment, VHH39 CDR2
LSWSNGGT
SEQ ID NO: 101-Anti-survivin binding fragment, VHH39 CDR3
STVVTRFGVKMFDV
SEQ ID NO: 102-Anti-survivin binding fragment, VHH39
QVQLVESGGGLVQAGESLRLSCATSARTINTYGLGWFRQAPGKGREFVAALSWSNG
GTRYADSVKGRFTVSRDNAKNTVYLQMNSLKPEDTAVYYCSTVVTRFGVKMFDV
WGRGTQVTVSS
SEQ ID NO: 103-Anti-survivin binding fragment, VHH40 CDR1
GFTFRNYW
SEQ ID NO: 104-Anti-survivin binding fragment, VHH40 CDR2
VGSDGRT
SEQ ID NO: 105-Anti-survivin binding fragment, VHH40 CDR3
YADQKGDY
SEQ ID NO: 106-Anti-survivin binding fragment, VHH40
QVKLEESGGGLVQPGGSLRLSCAASGFTFRNYWMYWVRQAPGKGLEWVARVGSD
GRTKYADSVKGRFTISKEAANNTLFLQMNSLKPEDTAVYYCYADQKGDYWSKGTQ
VTVSS
Survivin
SEQ ID NO: 201-Fragment (1) of human survivin
QNQPDLAECFFCFLQLQGWQPNN
SEQ ID NO: 202-Variant of SEQ ID NO: 1 with single amino acid change
QNQPDLAEMFFCFLQLQGWQPNN
SED ID NO: 203-Fragment of SEQ ID NO: 2
EMFFCF
SEQ ID NO: 204-Fragment of SEQ ID NO: 2 and comprises the sequence of
SEQ ID NO: 3
DLAEMFFCFLQLQGW
SEQ ID NO: 205-Fragment of SEQ ID NO: 2 and comprises the sequence of
SEQ ID NO: 3
AEMFFCFLQL
SEQ ID NO: 206-Fragment of SEQ ID NO: 2 and comprises the sequence of
SEQ ID NO: 3
EMFFCFLQL
SEQ ID NO: 207-Fragment (2) of human survivin
NLAECFFCFKQLQGW

1. An antibody, or an antigen-binding fragment thereof, wherein the antibody or antigen-binding fragment thereof comprises a heavy chain variable domain (VHH) comprising a complementarity-determining region (CDR) 1, a CDR2, and a CDR3, wherein:
i. CDR1 comprises the amino acid sequence of SEQ ID NO: 1, CDR2 comprises the amino acid sequence of SEQ ID NO: 2, and CDR3 comprises the amino acid sequence of SEQ ID NO: 3;
ii. CDR1 comprises the amino acid sequence of SEQ ID NO: 5, CDR2 comprises the amino acid sequence of SEQ ID NO: 6, and CDR3 comprises the amino acid sequence of SEQ ID NO: 7;
iii. CDR1 comprises the amino acid sequence of SEQ ID NO: 9, CDR2 comprises the amino acid sequence of SEQ ID NO: 2, and CDR3 comprises the amino acid sequence of SEQ ID NO: 10;
iv. CDR1 comprises the amino acid sequence of SEQ ID NO: 9, CDR2 comprises the amino acid sequence of SEQ ID NO: 12, and CDR3 comprises the amino acid sequence of SEQ ID NO: 13;
v. CDR1 comprises the amino acid sequence of SEQ ID NO: 15, CDR2 comprises the amino acid sequence of SEQ ID NO: 12, and CDR3 comprises the amino acid sequence of SEQ ID NO: 13;
vi. CDR1 comprises the amino acid sequence of SEQ ID NO: 1, CDR2 comprises the amino acid sequence of SEQ ID NO: 17, and CDR3 comprises the amino acid sequence of SEQ ID NO: 7;
vii. CDR1 comprises the amino acid sequence of SEQ ID NO: 1, CDR2 comprises the amino acid sequence of SEQ ID NO: 6, and CDR3 comprises the amino acid sequence of SEQ ID NO: 7;
viii. CDR1 comprises the amino acid sequence of SEQ ID NO: 1, CDR2 comprises the amino acid sequence of SEQ ID NO: 20, and CDR3 comprises the amino acid sequence of SEQ ID NO: 21;
ix. CDR1 comprises the amino acid sequence of SEQ ID NO: 23, CDR2 comprises the amino acid sequence of SEQ ID NO: 6, and CDR3 comprises the amino acid sequence of SEQ ID NO: 24;
x. CDR1 comprises the amino acid sequence of SEQ ID NO: 9, CDR2 comprises the amino acid sequence of SEQ ID NO: 26, and CDR3 comprises the amino acid sequence of SEQ ID NO: 27;
xi. CDR1 comprises the amino acid sequence of SEQ ID NO: 9, CDR2 comprises the amino acid sequence of SEQ ID NO: 6, and CDR3 comprises the amino acid sequence of SEQ ID NO: 29;
xii. CDR1 comprises the amino acid sequence of SEQ ID NO: 31, CDR2 comprises the amino acid sequence of SEQ ID NO: 32, and CDR3 comprises the amino acid sequence of SEQ ID NO: 33;
xiii. CDR1 comprises the amino acid sequence of SEQ ID NO: 35, CDR2 comprises the amino acid sequence of SEQ ID NO: 36, and CDR3 comprises the amino acid sequence of SEQ ID NO: 37;
xiv. CDR1 comprises the amino acid sequence of SEQ ID NO: 39, CDR2 comprises the amino acid sequence of SEQ ID NO: 40, and CDR3 comprises the amino acid sequence of SEQ ID NO: 37;
xv. CDR1 comprises the amino acid sequence of SEQ ID NO: 35, CDR2 comprises the amino acid sequence of SEQ ID NO: 42, and CDR3 comprises the amino acid sequence of SEQ ID NO: 37;
xvi. CDR1 comprises the amino acid sequence of SEQ ID NO: 39, CDR2 comprises the amino acid sequence of SEQ ID NO: 42, and CDR3 comprises the amino acid sequence of SEQ ID NO: 37;
xvii. CDR1 comprises the amino acid sequence of SEQ ID NO: 39, CDR2 comprises the amino acid sequence of SEQ ID NO: 40, and CDR3 comprises the amino acid sequence of SEQ ID NO: 45;
xviii. CDR1 comprises the amino acid sequence of SEQ ID NO: 47, CDR2 comprises the amino acid sequence of SEQ ID NO: 42, and CDR3 comprises the amino acid sequence of SEQ ID NO: 45;
xix. CDR1 comprises the amino acid sequence of SEQ ID NO: 49, CDR2 comprises the amino acid sequence of SEQ ID NO: 42, and CDR3 comprises the amino acid sequence of SEQ ID NO: 45;
xx. CDR1 comprises the amino acid sequence of SEQ ID NO: 51, CDR2 comprises the amino acid sequence of SEQ ID NO: 42, and CDR3 comprises the amino acid sequence of SEQ ID NO: 45;
xxi. CDR1 comprises the amino acid sequence of SEQ ID NO: 53, CDR2 comprises the amino acid sequence of SEQ ID NO: 54, and CDR3 comprises the amino acid sequence of SEQ ID NO: 55;
xxii. CDR1 comprises the amino acid sequence of SEQ ID NO: 57, CDR2 comprises the amino acid sequence of SEQ ID NO: 54, and CDR3 comprises the amino acid sequence of SEQ ID NO: 55;
xxiii. CDR1 comprises the amino acid sequence of SEQ ID NO: 9, CDR2 comprises the amino acid sequence of SEQ ID NO: 54, and CDR3 comprises the amino acid sequence of SEQ ID NO: 55;
xxiv. CDR1 comprises the amino acid sequence of SEQ ID NO: 60, CDR2 comprises the amino acid sequence of SEQ ID NO: 42, and CDR3 comprises the amino acid sequence of SEQ ID NO: 55;
xxv. CDR1 comprises the amino acid sequence of SEQ ID NO: 62, CDR2 comprises the amino acid sequence of SEQ ID NO: 42, and CDR3 comprises the amino acid sequence of SEQ ID NO: 55;
xxvi. CDR1 comprises the amino acid sequence of SEQ ID NO: 64, CDR2 comprises the amino acid sequence of SEQ ID NO: 42, and CDR3 comprises the amino acid sequence of SEQ ID NO: 55;
xxvii. CDR1 comprises the amino acid sequence of SEQ ID NO: 47, CDR2 comprises the amino acid sequence of SEQ ID NO: 42, and CDR3 comprises the amino acid sequence of SEQ ID NO: 55;
xxviii. CDR1 comprises the amino acid sequence of SEQ ID NO: 35, CDR2 comprises the amino acid sequence of SEQ ID NO: 42, and CDR3 comprises the amino acid sequence of SEQ ID NO: 55;
xxix. CDR1 comprises the amino acid sequence of SEQ ID NO: 68, CDR2 comprises the amino acid sequence of SEQ ID NO: 42, and CDR3 comprises the amino acid sequence of SEQ ID NO: 55;
xxx. CDR1 comprises the amino acid sequence of SEQ ID NO: 49, CDR2 comprises the amino acid sequence of SEQ ID NO: 42, and CDR3 comprises the amino acid sequence of SEQ ID NO: 55;
xxxi. CDR1 comprises the amino acid sequence of SEQ ID NO: 1, CDR2 comprises the amino acid sequence of SEQ ID NO: 20, and CDR3 comprises the amino acid sequence of SEQ ID NO: 71;
xxxii. CDR1 comprises the amino acid sequence of SEQ ID NO: 73, CDR2 comprises the amino acid sequence of SEQ ID NO: 74, and CDR3 comprises the amino acid sequence of SEQ ID NO: 75;
xxxiii. CDR1 comprises the amino acid sequence of SEQ ID NO: 77, CDR2 comprises the amino acid sequence of SEQ ID NO: 78, and CDR3 comprises the amino acid sequence of SEQ ID NO: 79;
xxxiv. CDR1 comprises the amino acid sequence of SEQ ID NO: 81, CDR2 comprises the amino acid sequence of SEQ ID NO: 82, and CDR3 comprises the amino acid sequence of SEQ ID NO: 83;
xxxv. CDR1 comprises the amino acid sequence of SEQ ID NO: 31, CDR2 comprises the amino acid sequence of SEQ ID NO: 85, and CDR3 comprises the amino acid sequence of SEQ ID NO: 86;
xxxvi. CDR1 comprises the amino acid sequence of SEQ ID NO: 88, CDR2 comprises the amino acid sequence of SEQ ID NO: 78, and CDR3 comprises the amino acid sequence of SEQ ID NO: 89;
xxxvii. CDR1 comprises the amino acid sequence of SEQ ID NO: 91, CDR2 comprises the amino acid sequence of SEQ ID NO: 92, and CDR3 comprises the amino acid sequence of SEQ ID NO: 93;
xxxviii. CDR1 comprises the amino acid sequence of SEQ ID NO: 95, CDR2 comprises the amino acid sequence of SEQ ID NO: 96, and CDR3 comprises the amino acid sequence of SEQ ID NO: 97;
xxxix. CDR1 comprises the amino acid sequence of SEQ ID NO: 99, CDR2 comprises the amino acid sequence of SEQ ID NO: 100, and CDR3 comprises the amino acid sequence of SEQ ID NO: 101; or
xl. CDR1 comprises the amino acid sequence of SEQ ID NO: 103, CDR2 comprises the amino acid sequence of SEQ ID NO: 104, and CDR3 comprises the amino acid sequence of SEQ ID NO: 105.

2. The antibody, or antigen-binding fragment thereof, of claim 1, wherein the VHH comprises an amino acid sequence having at least 90%, 91%, 92%, 93%, 94%, 95%, 96%, 97%, 98%, or 99% sequence identity to SEQ ID NO: 4, SEQ ID NO: 8, SEQ ID NO: 11, SEQ ID NO: 14, SEQ ID NO: 16, SEQ ID NO: 18, SEQ ID NO: 19, SEQ ID NO: 22, SEQ ID NO: 25, SEQ ID NO: 28, SEQ ID NO: 30, SEQ ID NO: 34, SEQ ID NO: 38, SEQ ID NO: 41, SEQ ID NO: 43, SEQ ID NO: 44, SEQ ID NO: 46, SEQ ID NO: 48, SEQ ID NO: 50, SEQ ID NO: 52, SEQ ID NO: 56, SEQ ID NO: 58, SEQ ID NO: 59, SEQ ID NO: 61, SEQ ID NO: 63, SEQ ID NO: 65, SEQ ID NO: 66, SEQ ID NO: 67, SEQ ID NO: 69, SEQ ID NO: 70, SEQ ID NO: 72, SEQ ID NO: 76, SEQ ID NO: 80, SEQ ID NO: 84, SEQ ID NO: 87, SEQ ID NO: 90, SEQ ID NO: 94, SEQ ID NO: 98, SEQ ID NO: 102, or SEQ ID NO: 106, provided that the VHH comprises the sequences of at least one set of CDR1, CDR2, and CDR3 sequences of claim 1.

3. The antibody, or antigen-binding fragment thereof, of claim 1, wherein the VHH comprises an amino acid sequence identical to SEQ ID NO: 4, SEQ ID NO: 8, SEQ ID NO: 11, SEQ ID NO: 14, SEQ ID NO: 16, SEQ ID NO: 18, SEQ ID NO: 19, SEQ ID NO: 22, SEQ ID NO: 25, SEQ ID NO: 28, SEQ ID NO: 30, SEQ ID NO: 34, SEQ ID NO: 38, SEQ ID NO: 41, SEQ ID NO: 43, SEQ ID NO: 44, SEQ ID NO: 46, SEQ ID NO: 48, SEQ ID NO: 50, SEQ ID NO: 52, SEQ ID NO: 56, SEQ ID NO: 58, SEQ ID NO: 59, SEQ ID NO: 61, SEQ ID NO: 63, SEQ ID NO: 65, SEQ ID NO: 66, SEQ ID NO: 67, SEQ ID NO: 69, SEQ ID NO: 70,


SEQUENCE LISTING (207 sequences)

SEQ ID NO: 1 - protein, 8 aa, synthetic construct
GVTFSTLT

SEQ ID NO: 2 - protein, 8 aa, synthetic construct
IKRTSGST

SEQ ID NO: 3 - protein, 22 aa, synthetic construct
AAARPFGYGSPRPRTGVDEYEY

SEQ ID NO: 4 - protein, 129 aa, synthetic construct
EVQLVESGGGLVQAGGSLGLSCAASGVTFSTLTMAWFRQASGKEREFVAAIKRTSGSTHYTDSVKGRFTISRDNAKNTVYLQMNSLKPEDTAVYYCAAARPFGYGSPRPRTGVDEYEYWGQGTQVTVSS

SEQ ID NO: 5 - protein, 8 aa, synthetic construct
GLTFTAYT

SEQ ID NO: 6 - protein, 8 aa, synthetic construct
IKRSGGST

SEQ ID NO: 7 - protein, 22 aa, synthetic construct
AAARPFGYGSRRPRTGVDEYDY

SEQ ID NO: 8 - protein, 129 aa, synthetic construct
DVQLVESGGGLVQAGGSLRVSCAASGLTFTAYTMGWFRQAPGKEREFVAVIKRSGGSTYYADSVKGRFTISRDNAKNTVYLQMNSLKSEDTAVYYCAAARPFGYGSRRPRTGVDEYDYWGQGTQVTVSS

SEQ ID NO: 9 - protein, 8 aa, synthetic construct
GLTFSTYT

SEQ ID NO: 10 - protein, 22 aa, synthetic construct
AAAQVFGYGSRRPRTGVDEYDD

SEQ ID NO: 11 - protein, 129 aa, synthetic construct
QVQLVESGGGLVQAGGSLGLSCAASGLTFSTYTMAWFRQAPGKEREFVAAIKRTSGSTYYTDSVKGRFTISRDNAKNTVYLQMNSLKPADTAVYYCAAAQVFGYGSRRPRTGVDEYDDWGQGTQVTVSS

SEQ ID NO: 12 - protein, 8 aa, synthetic construct
IKRSGSST

SEQ ID NO: 13 - protein, 22 aa, synthetic construct
AAARPFGYGLWRPRTGVDEYEY

SEQ ID NO: 14 - protein, 129 aa, synthetic construct
AVQLVDSGGGLVQPGGSLRLSCAASGLTFSTYTMAWFRQAPGKEREFVAAIKRSGSSTYYPDSVKGRFTISRDNAKNTVYLQMNSLKPADTAVYYCAAARPFGYGLWRPRTGVDEYEYWGQGTQVTVSS

SEQ ID NO: 15 - protein, 8 aa, synthetic construct
GVTFSTYT

SEQ ID NO: 16 - protein, 129 aa, synthetic construct
AVQLVDSGGGLVQAGGSLGLSCAASGVTFSTYTMAWFRQAPGKEREFVAAIKRSGSSTYYPDSVKGRFTISRDNAKNTVYLQMNSLKPADTAVYYCAAARPFGYGLWRPRTGVDEYEYWGQGTQVTVSS

SEQ ID NO: 17 - protein, 8 aa, synthetic construct
IKRSGASV

SEQ ID NO: 18 - protein, 129 aa, synthetic construct
DVQLVESGGGLVQAGGSLRLSCAASGVTFSTLTMGWFRQTPGKERVFVAAIKRSGASVYYADSVKGRFTISRDNAENTVYLQMISLKPEDTAVYYCAAARPFGYGSRRPRTGVDEYDYWGQGTQVTVSS

SEQ ID NO: 19 - protein, 129 aa, synthetic construct
DVQLVESGGGLVQAGGSLRVSCAASGLTFSAYTMGWFRQAPGKERDFVAAIKRSGGSTFYADSVKGRFTISRDNAKNMVYLQMNSLKPEDTAVYYCAAARPFGYGSRRPRTGVDEYDYWGQGTQVTVSS

SEQ ID NO: 20 - protein, 8 aa, synthetic construct
IKRSGGSV

SEQ ID NO: 21 - protein, 22 aa, synthetic construct
AAARPFGYGSRSPRTGVDEYDY

SEQ ID NO: 22 - protein, 129 aa, synthetic construct
QVKLEESGGGLVQAGGSLRLSCTASGVTFSTLTMGWFRQAPGKERVFVAAIKRSGGSVYYADSVKGRFTISRDNAKNTVYLQMNSLKPEDTAVYYCAAARPFGYGSRSPRTGVDEYDYWGQGTQVTVSS

SEQ ID NO: 23 - protein, 8 aa, synthetic construct
GLTFSTLT

SEQ ID NO: 24 - protein, 22 aa, synthetic construct
AAARPFGYGSWRPRTGVDEYDY

SEQ ID NO: 25 - protein, 129 aa, synthetic construct
AVQLVESGGGLVQAGGSLRVSCAASGLTFSTLTMGWFRQAPGKEREFVAAIKRSGGSTYYADSVKGRFTISRDNAKNTVYLQMNSLKPEDTAVYYCAAARPFGYGSWRPRTGVDEYDYWGQGTQVTVSS

SEQ ID NO: 26 - protein, 8 aa, synthetic construct
LTRTGRNT

SEQ ID NO: 27 - protein, 22 aa, synthetic construct
AAARVFGYGSPRPRTGVDEYDY

SEQ ID NO: 28 - protein, 129 aa, synthetic construct
DVQLVESGGGLVQAGGSLRLSCAASGLTFSTYTMGWFRQAPGKEREIVAALTRTGRNTYYADSVEGRFTISRDNAKNTVYLQMNSLKPEDTAVYYCAAARVFGYGSPRPRTGVDEYDYWGQGTQVTVSS

SEQ ID NO: 29 - protein, 22 aa, synthetic construct
AAARVLGYGSRRPRTGVDEYDY

SEQ ID NO: 30 - protein, 129 aa, synthetic construct
AVQLVDSGGGLVQAGGSLRVSCAASGLTFSTYTMGWFRQAPGKERDFVAAIKRSGGSTYYADSVKGRFTISRDNAKNTVYLQMNSLKPEDTAVYYCAAARVLGYGSRRPRTGVDEYDYWGQGTQVTVSS

SEQ ID NO: 31 - protein, 8 aa, synthetic construct
GRTFSSYV

SEQ ID NO: 32 - protein, 8 aa, synthetic construct
IKWMGGTT

SEQ ID NO: 33 - protein, 17 aa, synthetic construct
AARLITGRPSPRGDYDY

SEQ ID NO: 34 - protein, 124 aa, synthetic construct
QVQLVESGGGLVQAGGSLRLSCAASGRTFSSYVMAWFRQAPGEAREFIAAIKWMGGTTSYAESVKGRFTISRDKAMNTVYLQMNSLKPEDTAVYYCAARLITGRPSPRGDYDYWGQGTRVTVSA

SEQ ID NO: 35 - protein, 8 aa, synthetic construct
GRSFSSMS

SEQ ID NO: 36 - protein, 7 aa, synthetic construct
ISPSGKT

SEQ ID NO: 37 - protein, 21 aa, synthetic construct
AASRRTYYSATPRRTEEMYDY

SEQ ID NO: 38 - protein, 127 aa, synthetic construct
QVKLEESGGGLVQAGDSLRLSCAASGRSFSSMSMGWFRQAPGTQREFVATISPSGKTYYADSVKGRFTISRENAKDTVYLQMNSLKPEDTAVYYCAASRRTYYSATPRRTEEMYDYWGQGTQVTVSS

SEQ ID NO: 39 - protein, 8 aa, synthetic construct
GRTFSSMS

SEQ ID NO: 40 - protein, 7 aa, synthetic construct
ITPSGKT

SEQ ID NO: 41 - protein, 127 aa, synthetic construct
AVQLVDSGGGLVQAGDSLRLSCAVSGRTFSSMSMGWFRQAPGKQREFVATITPSGKTYYADSVKGRFTISRENAKDTVYLQMNSLKPEDTAVYYCAASRRTYYSATPRRTEEMYDYWGQGTQVTVSS

SEQ ID NO: 42 - protein, 7 aa, synthetic construct
ITPSGRT

SEQ ID NO: 43 - protein, 127 aa, synthetic construct
AVQLVDSGGGLVQAGDSLRLSCAASGRSFSSMSMGWFRQAPGTQREFVATITPSGRTYYADSVKGRFTISRENAKDTVYLQMNSLKPEDTAVYYCAASRRTYYSATPRRTEEMYDYWGQGTQVTVSS

SEQ ID NO: 44 - protein, 127 aa, synthetic construct
QVQLVESGGGLVQSGGSLRLPCAASGRTFSSMSMGWFRQAPGTQREFVATITPSGRTYYADSVKGRFTISRENAKDTVYLQMNSLKPEDTAVYYCAASRRTYYSATPRRTEEMYDYWGQGTQVTVSS

SEQ ID NO: 45 - protein, 21 aa, synthetic construct
AASRRTYYSATPRRTGEMYDY

SEQ ID NO: 46 - protein, 127 aa, synthetic construct
EVQLVESGGGLVQAGDSLRLSCAVSGRTFSSMSMGWFRQAPGKQREFVATITPSGKTYYADSVKGRFTISRENAKDTVYLQMNSLKPEDTAVYYCAASRRTYYSATPRRTGEMYDYWGQGTQVTVSS

SEQ ID NO: 47 - protein, 8 aa, synthetic construct
GRAFSSYS

SEQ ID NO: 48 - protein, 127 aa, synthetic construct
AVQLVDSGGGLVQAGGSLRLSCATSGRAFSSYSMGWFRQAPGKEREFVATITPSGRTHYADSVKGRFTISRENAKDTVYLQMNSLKPADTAVYYCAASRRTYYSATPRRTGEMYDYWGQGTQVTVSS

SEQ ID NO: 49 - protein, 8 aa, synthetic construct
GRTFSSYS

SEQ ID NO: 50 - protein, 127 aa, synthetic construct
AVQLVESGGGLVQPGGSLRLSCAVSGRTFSSYSMGWFRQAPGKEREFVATITPSGRTYYADSVKGRFTISRENAKDTVYLQMNSLKPEDTAVYYCAASRRTYYSATPRRTGEMYDYWGQGTQVTVSS

SEQ ID NO: 51 - protein, 8 aa, synthetic construct
GRTGSSYS

SEQ ID NO: 52 - protein, 127 aa, synthetic construct
AVQLVDSGGGLVQAGGSLRVSCAASGRTGSSYSWGWFRQAPGKEREFVATITPSGRTYYPDSVKGRFTISRENAKDTVYLQMNSLKPEDTAVYYCAASRRTYYSATPRRTGEMYDYWGQGTQVTVSS

SEQ ID NO: 53 - protein, 8 aa, synthetic construct
GGAFSSYS

SEQ ID NO: 54 - protein, 7 aa, synthetic construct
ITPRGRT

SEQ ID NO: 55 - protein, 21 aa, synthetic construct
AASRRTYYSPTPRRTGEMYDY

SEQ ID NO: 56 - protein, 127 aa, synthetic construct
QVQLVESGGGLVQAGGSLRLSCAAAGGAFSSYSMGWFRQAPGKEREFVATITPRGRTYSSDSVKGRFTISRENAEDTVYLQMNSLKPEDTAVYYCAASRRTYYSPTPRRTGEMYDYWGQGTQVTVSS

SEQ ID NO: 57 - protein, 8 aa, synthetic construct
GGAFSTYS

SEQ ID NO: 58 - protein, 127 aa, synthetic construct
QVQLVESGGGLVQPGDSLRLSCAAAGGAFSTYSMGWFRQAPGKEREFVATITPRGRTYYSDSVKGRFTISRENAEDTVYLQMNSLKPEDTAVYYCAASRRTYYSPTPRRTGEMYDYWGQGTQVTVSS

SEQ ID NO: 59 - protein, 127 aa, synthetic construct
DVQLVESGGGLVQAGGSLRLSCAASGLTFSTYTMGWFRQAPGKEREFVATITPRGRTYYSDSVKGRFTISRENAEDTVYLQMNSLKPEDTAAYYCAASRRTYYSPTPRRTGEMYDYWGQGTQVTVSS

SEQ ID NO: 60 - protein, 8 aa, synthetic construct
ERAFSSYS

SEQ ID NO: 61 - protein, 127 aa, synthetic construct
DVQLVESGGGLVQAGGSLRLSCAASERAFSSYSMGWFRQAPGKEREFVATITPSGRTYYPDSVKGRFTISRENVKDTVYLQMNSLKPEDTAVYYCAASRRTYYSPTPRRTGEMYDYWGQGTQVTVSS

SEQ ID NO: 62 - protein, 8 aa, synthetic construct
ERTFSSYS

SEQ ID NO: 63 - protein, 127 aa, synthetic construct
EVQLVESGGGLVQSGGSLRLSCAASERTFSSYSMGWFRQAPGKEREFVATITPSGRTYYPDSVKGRFTISRENVKDTVYLQMNSLKPEDTAVYYCAASRRTYYSPTPRRTGEMYDYWGQGTQVTVSS

SEQ ID NO: 64 - protein, 8 aa, synthetic construct
GGTFSSYS

SEQ ID NO: 65 - protein, 127 aa, synthetic construct
DVQLVESGGGLVQPGGSLRLSCAASGGTFSSYSMGWFRQAPGKEREFVATITPSGRTYYADSMRGRFTISREGAKDTVYLQMNDLKPEDTAVYYCAASRRTYYSPTPRRTGEMYDYWGQGTQVTVSS

SEQ ID NO: 66 - protein, 127 aa, synthetic construct
EVQLVESGGGLVQTGGSLRLSCAVSGRAFSSYSMGWFRQAPGKERVFVATITPSGRTYYSDSVKGRFTISRENAEDTVYLQMNSLKPEDTAVYYCAASRRTYYSPTPRRTGEMYDYWGQGTHVTVSS

SEQ ID NO: 67 - protein, 127 aa, synthetic construct
AVQLVDSGGGLVQAGDSLRLSCAASGRSFSSMSMGWFRQAPGKEREFVATITPSGRTYYPDSVKGRFTISRENVKDTVYLQMNSLKPEDTAVYYCAASRRTYYSPTPRRTGEMYDYWGQGTQVTVSS

SEQ ID NO: 68 - protein, 8 aa, synthetic construct
GRTFSSFS

SEQ ID NO: 69 - protein, 127 aa, synthetic construct
AVQLVDSGGGLVQPGGSLRLSCAASGRTFSSFSMGWFRQAPGKEREFVATITPSGRTYYSDSVKGRFTISRENPEATVYLQMNSLKPEDTAVYYCAASRRTYYSPTPRRTGEMYDYWGQGTQVTVSS

SEQ ID NO: 70 - protein, 127 aa, synthetic construct
QVKLEESGGGLVQAGGSLTLSCAVSGRTFSSYSMGWFRQAPGKEREFVATITPSGRTYHANSVKGRFTISRENAKDTVYLQMNSLKPEDTAVYYCAASRRTYYSPTPRRTGEMYDYWGQGTQVTVSS

SEQ ID NO: 71 - protein, 22 aa, synthetic construct
AAVRPFGYGSRSPRTGVDEYDY

SEQ ID NO: 72 - protein, 129 aa, synthetic construct
AVQLVESGGGLVQAGESLRLSCTASGVTFSTLTMGWFRQAPGKERVFVAAIKRSGGSVYYADSVKGRFTISRDNAKNTVYLQMNSLKPEDTAVYYCAAVRPFGYGSRSPRTGVDEYDYWGQGTQVTVSS

SEQ ID NO: 73 - protein, 8 aa, synthetic construct
GRTFSSYN

SEQ ID NO: 74 - protein, 8 aa, synthetic construct
ISRSGSRT

SEQ ID NO: 75 - protein, 22 aa, synthetic construct
AADDRRYRNSVSTPPNPSDYDY

SEQ ID NO: 76 - protein, 129 aa, synthetic construct
EVQLVESGGGLVQAGGSLRLSCAASGRTFSSYNMGWFRQAPGKEREYVAAISRSGSRTYYLNSVKDRFTISRDNAKNTVYLQMNSLKPEDTAVYYCAADDRRYRNSVSTPPNPSDYDYWGQGTQVTVSS

SEQ ID NO: 77 - protein, 8 aa, synthetic construct
GRTFGINV

SEQ ID NO: 78 - protein, 7 aa, synthetic construct
KSSGGST

SEQ ID NO: 79 - protein, 13 aa, synthetic construct
AAGTTWGQWRYRY

SEQ ID NO: 80 - protein, 119 aa, synthetic construct
QVKLEESGGGLVQPGGSLSLSCAASGRTFGINVMGWYRQAPGKQRELVAAKSSGGSTRYADSVKGRFTISRDNAKNTVYLQMNSLKPEDTAVYYCAAGTTWGQWRYRYWGQGTQVTVSS

SEQ ID NO: 81 - protein, 8 aa, synthetic construct
GRAFSTYN

SEQ ID NO: 82 - protein, 8 aa, synthetic construct
INWSGVTT

SEQ ID NO: 83 - protein, 23 aa, synthetic construct
AAKAKNWVWARSRPAFSSSEYDY

SEQ ID NO: 84 - protein, 130 aa, synthetic construct
AVQLVESGGGLVQAGGSLRLTCAASGRAFSTYNMAWFRQAPGKEREFVAAINWSGVTTYYGDSVKGRFTISRDNAKNTVYLQMNSLKSEDTAVYYCAAKAKNWVWARSRPAFSSSEYDYWGQGTQVTVSS

SEQ ID NO: 85 - protein, 8 aa, synthetic construct
ISWFGGST

SEQ ID NO: 86 - protein, 20 aa, synthetic construct
AAKPRGGTSAQAMEVMYYDY

SEQ ID NO: 87 - protein, 127 aa, synthetic construct
QVKLEESGGGLVQAGGSLRLSCAASGRTFSSYVMGWFRQAPGKEREFVAAISWFGGSTRYADSVKGRFTISRDNAKNTVFLQMNTLKPEDTAVYYCAAKPRGGTSAQAMEVMYYDYWGQGTQVTVSS

SEQ ID NO: 88 - protein, 8 aa, synthetic construct
GRTFSINV

SEQ ID NO: 89 - protein, 15 aa, synthetic construct
AATPRGAYYNAQYRY

SEQ ID NO: 90 - protein, 121 aa, synthetic construct
QVQLQESGGGSVQPGGSLSLSCAASGRTFSINVMGWYRQAPGKQRELVAAKSSGGSTRYADSVKGRFTISRDNAKNTVYLQMNNLKPEDTAVYYCAATPRGAYYNAQYRYWGQGTQVTVSS

SEQ ID NO: 91 - protein, 8 aa, synthetic construct
GRTFSSYA

SEQ ID NO: 92 - protein, 8 aa, synthetic construct
INSSGGSI

SEQ ID NO: 93 - protein, 10 aa, synthetic construct
NYVRITAVNY

SEQ ID NO: 94 - protein, 117 aa, synthetic construct
EVQLVESGGGLVQAGGSLRLSCAASGRTFSSYAMGWFRQAPGKGRDFVGAINSSGGSILYADSVKGRFTISRDNAKNTVFLQMNSLEPEDTAVYYCNYVRITAVNYWGQGTQVTVSS

SEQ ID NO: 95 - protein, 8 aa, synthetic construct
PNAFSSYA

SEQ ID NO: 96 - protein, 8 aa, synthetic construct
ISPSGGYI

SEQ ID NO: 97 - protein, 9 aa, synthetic construct
SGGRNSWDN

SEQ ID NO: 98 - protein, 116 aa, synthetic construct
VQLVESGGGLVQTGGSLRLSCAASGPNAFSSYAMGWFRQRPGKEREFVAAISPSGGYIQYEDSVKGRSTISRDNSKNMMYLQMVNLKPDDTAVYYCSGGRNSWDNWGQGIQVTVSS

SEQ ID NO: 99 - protein, 8 aa, synthetic construct
ARTINTYG

SEQ ID NO: 100 - protein, 8 aa, synthetic construct
LSWSNGGT

SEQ ID NO: 101 - protein, 14 aa, synthetic construct
STVVTRFGVKMFDV

SEQ ID NO: 102 - protein, 121 aa, synthetic construct
QVQLVESGGGLVQAGESLRLSCATSARTINTYGLGWFRQAPGKGREFVAALSWSNGGTRYADSVKGRFTVSRDNAKNTVYLQMNSLKPEDTAVYYCSTVVTRFGVKMFDVWGRGTQVTVSS

SEQ ID NO: 103 - protein, 8 aa, synthetic construct
GFTFRNYW

SEQ ID NO: 104 - protein, 7 aa, synthetic construct
VGSDGRT

SEQ ID NO: 105 - protein, 8 aa, synthetic construct
YADQKGDY

SEQ ID NO: 106 - protein, 114 aa, synthetic construct
QVKLEESGGGLVQPGGSLRLSCAASGFTFRNYWMYWVRQAPGKGLEWVARVGSDGRTKYADSVKGRFTISKEAANNTLFLQMNSLKPEDTAVYYCYADQKGDYWSKGTQVTVSS

SEQ ID NO: 107-200 - skipped (94 entries)

SEQ ID NO: 201 - protein, 23 aa, Homo sapiens
QNQPDLAECFFCFLQLQGWQPNN

SEQ ID NO: 202 - protein, 23 aa, synthetic construct
QNQPDLAEMFFCFLQLQGWQPNN

SEQ ID NO: 203 - protein, 6 aa, synthetic construct
EMFFCF

SEQ ID NO: 204 - protein, 15 aa, synthetic construct
DLAEMFFCFLQLQGW

SEQ ID NO: 205 - protein, 10 aa, synthetic construct
AEMFFCFLQL

SEQ ID NO: 206 - protein, 9 aa, synthetic construct
EMFFCFLQL

SEQ ID NO: 207 - protein, 15 aa, Homo sapiens
NLAECFFCFKQLQGW